(19) United States
(12) Patent Application Publication
(10) Pub. No.: US 2023/0002838 A1
(43) Pub. Date: Jan. 5, 2023

(54) Improved Methods for the Production of Plants

(71) Applicant: Agriculture Victoria Services Pty Ltd, Bundoora (AU)

(72) Inventors: Noel Cogan, Macleod (AU); Shivraj Kaur Braich, Glenroy (AU); Larry Stephen Jewell, Eltham (AU); German Carlos Spangenberg, Bundoora (AU); Rebecca C. Baillie, Hurstbridge (AU)

(21) Appl. No.: 17/627,402

(22) PCT Filed: Jul. 31, 2020
(86) PCT No.: PCT/AU2020/050792

§ 371 Date: Jan. 14, 2022

(30) Foreign Application Priority Data
Aug. 1, 2019 (AU) 2019902745
Aug. 8, 2019 (AU) 2019902844

(51) Int. Cl. C12Q 1/6895; A01H 1/04; A01H 6/28

(52) U.S. Cl. CPC C12Q 1/6895; A01H 1/04; A01H 6/28; C12Q 2600/13

(57) ABSTRACT

The present disclosure relates generally to methods useful to the production of cannabis plants, including methods for determining the sex of a cannabis plant, methods for determining the developmental stage of a female cannabis plant inflorescence, methods for monitoring the development of female cannabis plant inflorescence, methods for standardising the harvesting of female cannabis plants, methods for selecting a female cannabis plant for harvest and methods for selecting a hypoallergenic cannabis plant.

### FIELD

The present disclosure relates generally to the production of cannabis plants, including methods for sex determination and monitoring of inflorescence development based on transcriptional changes that occur during the development of cannabis plants.

### RELATED APPLICATIONS

This application claims priority from Australian Provisional Patent Application No. 2019902745 filed 1 Aug. 2019 and Australian Provisional Patent Application No. 2019902844 filed 8 Aug. 2019, the entire content of which are hereby incorporated by reference.

### BACKGROUND

Cannabis is an herbaceous flowering plant of the Cannabis genus (Rosale), which has been used for its fiber and medicinal properties for thousands of years. The medicinal qualities of cannabis have been recognised since at least 2800 BC, with use of cannabis featuring in ancient Chinese and Indian medical texts. Although the use of cannabis for medicinal purposes has been known for centuries, research into the pharmacological properties of the plant has been limited due to its illegal status in most jurisdictions.

The chemical profile of cannabis plants is varied. It is estimated that cannabis plants produce more than 400 different molecules, including phytocannabinoids, terpenes, and phenolics. Cannabinoids, such as Δ-9-tetrahydrocannabinol (THC) and cannabidiol (CBD), are typically the most commonly known and researched cannabinoids. CBD and THC are naturally present in their acidic forms, Δ-9-tetrahydrocannabinolic acid (THCA) and cannabidioloic acid (CBDA), which are alternative products of the same precursor, cannabigerolic acid (CBGA).

Despite advances in plant breeding technologies and the increasing commercial importance of cannabis plant varieties, there remains a need for improved methods of selected breeding of cannabis plants with one or more desirable phenotypic and/or chemotypic traits, including for large-scale production and breeding programs.

### SUMMARY

In an aspect disclosed herein, there is provided a method for determining the sex of a cannabis plant, the method comprising:
a. providing a nucleic acid sample obtained from cannabis plant tissue;
b. determining the level of expression of one or more Cannabis sativa genes, or homologs thereof, wherein the one or more genes encode a gene product selected from the group consisting of:
   i. lipoxygenase,
   ii. cannabinoid synthesis protein,
   iii. geranyl diphosphate pathway protein,
   iv. plastidial methyerythrito phosphate (MEP) pathway protein,
   v. terpene synthesis protein,
   vi. MADs box floral initiation transcription factor,
   vii. cannabis allergens, and
   viii. leucine-rich repeat (LRR) containing protein;
c. comparing the level of expression of the one or more genes determined in (b) with a sex determination reference value; and
d. determining the sex of the cannabis plant based on the comparison made in (c).

In another aspect disclosed herein, there is provided a method for determining the developmental stage of a female cannabis plant inflorescence, the method comprising:
a. providing a nucleic providing a nucleic sample obtained from female cannabis inflorescence or a part thereof;
b. determining the level of expression of one or more Cannabis sativa genes, or homologs thereof, wherein the one or more genes encode a gene product selected from the group consisting of:
   i. cannabinoid synthesis protein,
   ii. terpene synthesis protein,
   iii. MEP pathway protein,
   iv. cytostolic mevalonate (MEV) pathway protein, and
   v. MADs box floral initiation transcription factor;
c. comparing the level of expression of the one or more genes determined in (b) with a developmental reference value; and
d. determining the developmental stage of the inflorescence based on the comparison made in (c).

In another aspect disclosed herein, there is provided a method for monitoring the development of female cannabis plant inflorescence, the method comprising:
a. determining the developmental stage of a first inflorescence from a female cannabis plant in accordance with the methods disclosed herein;
b. determining the development stage of a second inflorescence from the plant of (a) in accordance with the methods disclosed herein at a subsequent time point in the growth cycle of the plant; and
c. comparing the developmental stage determined at (a) and (b) to evaluate whether there has been a change in the developmental stage of the inflorescence.

In another aspect disclosed herein, there is provided a method for standardising the harvesting of female cannabis plants, the method comprising:
a. determining the developmental stage of an inflorescence from a female cannabis plant in accordance with the methods disclosed herein;
b. determining the developmental stage of an inflorescence from one or more additional female cannabis plants in accordance with the methods disclosed herein;
c. comparing the developmental stage determined at (a) and (b) to evaluate if the female cannabis plants have inflorescence at the same developmental stage;
d. optionally, determining the developmental stage of additional inflorescence from the plants of (a) and (b) in accordance with the methods disclosed herein at a subsequent time point in the growth cycle of the plants; and
e. harvesting the plants when the inflorescence are determined to be at the same developmental stage.

In another aspect disclosed herein, there is provided a method for selecting a female cannabis plant for harvest, wherein the female cannabis plant produces inflorescence comprising a cannabinoid profile enriched for total CBD and total THC, the method comprising:
a. determining the developmental stage of an inflorescence from a female cannabis plant in accordance with the methods disclosed herein;
b. optionally, determining the developmental stage of an additional inflorescence from the plant of (a) in accordance with the methods disclosed herein at a subsequent time point in the growth cycle of the plant;
c. harvesting the plant when the inflorescence are determined to be at developmental Stage 4,
   wherein the cannabinoid profile comprises a level of total CBD and a level of total THC at a ratio of from about 1:1 to about 5:1 (CBD:THC), wherein the total CBD comprises cannabidiol (CBD) and/or cannabidiolic acid (CBDA), and wherein the total THC comprises Δ-9-tetrahydrocannabinol (THC) and/or Δ-9-tetrahydrocannabinolic acid (THCA), and wherein the level of total CBD and total THC (CBD+THC) is greater than the level of a reference cannabinoid selected from the group consisting of:
d. total CBC, wherein total CBC comprises cannabichromene (CBC) and/or cannabichromene acid (CBCA), and wherein CBD+THC is present at a ratio of from about 10:1 to about 50:1 to the level of total CBC (CBD+THC:CBC);
e. total CBG, wherein the total CBG comprises cannabigerol (CBG) and/or cannabigerolic acid (CBGA), and wherein CBD+THC is present at a ratio of from about 10:1 to about 110:1 to the level of total CBG (CBD+THC:CBG);
f. total CBN, wherein the total CBN comprises cannabinol (CBN) and/or cannabinolic acid (CBNA), and wherein CBD+THC is present at a ratio of from about 400:1 to about 4000:1 to the level of total CBN (CBD+THC:CBN);
g. total THCV, wherein the total THCV comprises tetrahydrocannabivarin (THCV) and/or tetrahydrocannabivarinic acid (THCVA), and wherein CBD+THC is present at a ratio of from about 100:1 to about 600:1 to the level of total THCV (CBD+THC:THCV); and
h. total CBDV, wherein the total CBDV comprises cannabidivarin (CBDV) and/or cannabidivarinic acid (CBDVA), and wherein CBD+THC is present at a ratio of from about 100:1 to about 2000:1 to the level of CBDV (CBD+THC:CBDV).

In another aspect disclosed herein, there is provided a method for selecting a hypoallergenic cannabis plant from a plurality of different cannabis plants, the method comprising:
a. providing a nucleic acid sample obtained from cannabis plant tissue;
b. determining the level of expression of one or more Cannabis sativa genes, or homologs thereof, wherein the one or more genes encode a cannabis allergen;
c. comparing the level of expression determined in (b) with an allergen reference value; and
d. selecting a hypoallergenic cannabis plant based on the comparison made in (c).

### BRIEF DESCRIPTION OF THE FIGURES

FIG. 1 is a graphical representation of the distribution of the contig and scaffold length (Transcript length (bp); x-axis) against the number of transcripts (y-axis) from the female cannabis transcriptome assembly.

FIG. 2 is a graphical representation of genus distribution of the female cannabis characterised transcripts based on UniRef100 annotation.

FIG. 3 is a graphical representation of the distribution of gene ontology (GO) terms (x-axis) against the percentage of specific category of genes present in each main category (left y-axis) or number of genes in the same category (right y-axis) for the female cannabis transcriptome. These results are summarised in three main categories of Cellular Component, Molecular Function and Biological Process.

FIG. 4 is a graphical representation of Principle Component Analysis (PCA) of transcriptome variation between various tissue types of female and male cannabis plants included in the assembly.

FIG. 5 is a graphical representation of the number of differentially expressed genes (y-axis) against various tissue types in male and female cannabis plants (x-axis). Black bars are representative of up-regulated genes; grey bars are representative of down-regulated genes.

FIG. 6 is a graphical representation of the number of differentially expressed genes (y-axis) against various developmental stages of flower development (x-axis) in the tissues of the (A) flower; and (B) trichomes of the female cannabis plant.

FIG. 7 is a graphical representation of differentially expressed transcripts of interest representing hierarchical clustering across (A) the various tissue types in male and female cannabis plants; and (B) the developmental stages in trichomes of the female cannabis plant. Normalised log transformed counts are indicated by the colour key. Grey represents high expression, white represents medium expression, and black represents low expression.

FIG. 8 is a photographic representation of the floral buds of a reproductive female cannabis plant at (A) 35 days; (B) 42 days; (C) 49 days; and (D) 56 days post-induction of flowering.

FIG. 9 is a graphical representation of summarised gene ontology (GO) terms related to biological processes, cellular component and molecular function of differentially expressed genes at developmental Stage 1 compared to Stage 4 in (A) flowers; and (B) trichomes. Circle size and shading is proportional to the log size of the GO terms, shade indicates the uniqueness. Distance between circles is representative of GO terms' semantic similarities. Each of the circles represents a GO term, which, depending on the similarity in the terms included in them, they will be closer or more distant in the graph.

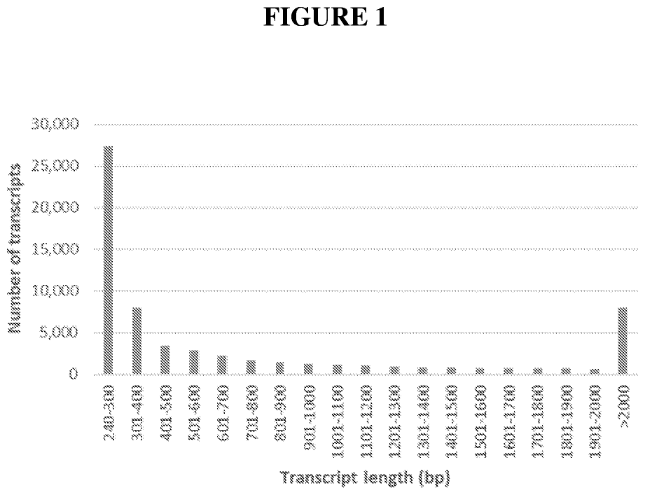

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgement or admission or any form of suggestion that that prior publication (or information derived from it) or known matter forms part of the common general knowledge in the field of endeavour to which this specification relates.

Unless specifically defined otherwise, all technical and scientific terms used herein shall be taken to have the same meaning as commonly understood by one of ordinary skill in the art.

Unless otherwise indicated the molecular biology, cell culture, laboratory, plant breeding and selection techniques utilised in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbor Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present); Janick, J. (2001) Plant Breeding Reviews, John Wiley & Sons, 252 p.; Jensen, N. F. ed. (1988) Plant Breeding Methodology, John Wiley & Sons, 676 p., Richard, A. J. ed. (1990) Plant Breeding Systems, Unwin Hyman, 529 p.; Walter, F. R. ed. (1987) Plant Breeding, Vol. I, Theory and Techniques, MacMillan Pub. Co.; Slavko, B. ed. (1990) Principles and Methods of Plant Breeding, Elsevier, 386 p.; and Allard, R. W. ed. (1999) Principles of Plant Breeding, John-Wiley & Sons, 240 p. The ICAC Recorder, Vol. XV no. 2: 3-14; all of which are incorporated by reference. The procedures described are believed to be well known in the art and are provided for the convenience of the reader. All other publications mentioned in this specification are also incorporated by reference in their entirety.

As used in the subject specification, the singular forms “a”, “an” and “the” include plural aspects unless the context clearly dictates otherwise. Thus, for example, reference to “a plant” includes a single plant, as well as two or more plants; reference to “an ortholog” includes a single ortholog, as well as two or more orthologs; and so forth.

The present disclosure is predicated, at least in part, on the unexpected finding that cannabis plants have distinct gene expression profiles that can be used to accurately distinguish between male and female cannabis plants and the developmental stage of a female cannabis plant inflorescence. Such gene expression profiles may be used in advantageous plant production methods, examples of which include optimisation of harvest time for maximum resin production or sex determination at early stages of plant development.

### Cannabis

As used herein, the term “cannabis plant” means a plant of the genus Cannabis, illustrative examples of which include Cannabis sativa, Cannabis indica and Cannabis ruderalis. Cannabis is an erect annual herb with a dioecious breeding system, although monoecious plants exist. Wild and cultivated forms of cannabis are morphologically variable, which has resulted in difficulty defining the taxonomic organisation of the genus. In an embodiment, the cannabis plant is Cannabis sativa, also referred to as C. sativa.

The terms “plant”, “cultivar”, “variety”, “strain” or “race” are used interchangeably herein to refer to a plant or a group of similar plants according to their structural features and performance (i.e., morphological and physiological characteristics).

The reference genome for C. sativa is the assembled draft genome and transcriptome of “Purple Kush” or “PK” (van Bakal et al. supra). C. sativa, has a diploid genome (2n=20) with a karyotype comprising nine autosomes and a pair of sex chromosomes (X and Y). Female plants are homogametic (XX) and males heterogametic (XY) with sex determination controlled by an X-to-autosome balance system. The estimated size of the haploid genome is 818 Mb for female plants and 843 Mb for male plants.

As used herein, the term “plant part” refers to any part of the plant, illustrative examples of which include an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen. The term “plant part” also includes any material listed in the Plant Part Code Table as approved by the Australian Therapeutic Goods Administration (TGA) Business Services (TBS). In an embodiment, the part is selected from the group consisting of an embryo, a shoot, a bud, a root, a stem, a seed, a stipule, a leaf, a petal, an inflorescence, an ovule, a bract, a trichome, a branch, a petiole, an internode, bark, a pubescence, a tiller, a rhizome, a frond, a blade, pollen and stamen.

### Cannabinoids

The term “cannabinoid”, as used herein, refers to a family of terpeno-phenolic compounds, of which more than 100 compounds are known to exist in nature. Cannabinoids will be known to persons skilled in the art, illustrative examples of which are provided in Table 1, below, including acidic and decarboxylated forms thereof.

TABLE 1
Cannabinoids and their properties.

| | | Chemical |
|---|---|---|
| | | properties/ |
| | | [M + H]⁺ ESI |
| Name | Structure | MS |
| Δ9- tetrahydrocannabinol (THC) | 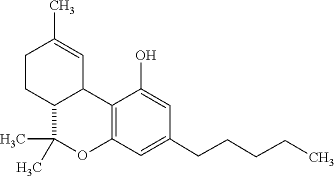 | Psychoactive, decarboxylation product of THCA m/z 315.2319 |
| Δ9- tetrahydrocannabinolic acid (THCA) | 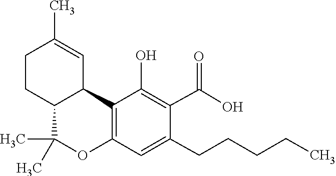 [SMILES: CCCCCc1cc2c(c(O)c1C(=O)O)[C@@H]1C=C(C)CC[C@H]1C(C)(C)O2] | m/z 359.2217 |
| cannabidiol (CBD) | 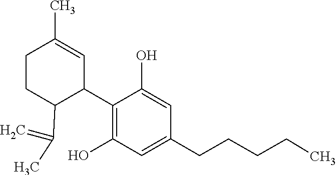 [SMILES: C=C(C)C1CCC(C)=CC1c1c(O)cc(CCCCC)cc1O] | decarboxylation product of CBDA m/z 315.2319 |
| cannabidiolic acid (CBDA) | 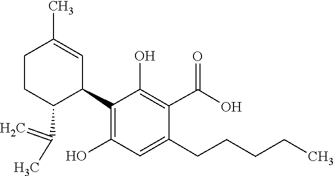 [SMILES: C=C(C)[C@@H]1CCC(C)=C[C@H]1c1c(O)cc(CCCCC)c(C(=O)O)c1O] | m/z 359.2217 |
| cannabigerol (CBG) | 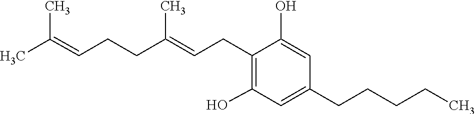 [SMILES: CCCCCc1cc(O)c(C/C=C(\C)CCC=C(C)C)c(O)c1] | Non- intoxicating, decarboxylation product of CBGA m/z 317.2475 |
| cannabigerolic acid (CBGA) | 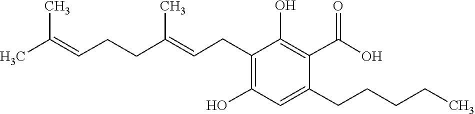 [SMILES: CCCCCc1cc(O)c(C/C=C(\C)CCC=C(C)C)c(O)c1C(=O)O] | m/z 361.2373 |
| cannabichromene (CBC) | 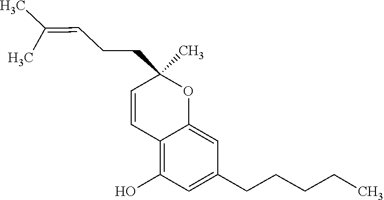 [SMILES: CCCCCc1cc(O)c2c(c1)O[C@](C)(CCC=C(C)C)C=C2] | Non- psychotropic, converts to cannabicyclol upon light exposure m/z 315.2319 |
| cannabichromene acid (CBCA) | 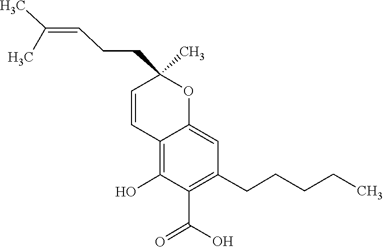 [SMILES: CCCCCc1cc2c(c(O)c1C(=O)O)C=C[C@@](C)(CCC=C(C)C)O2] | m/z 359.2217 |
| cannabicyclol (CBL) | 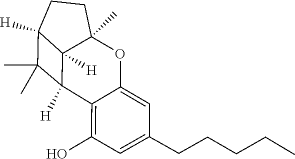 | Non- psychoactive, 16 isomers known. Derived from non- enzymatic conversion of CBC m/z 315.2319 |
| cannabinol (CBN) | 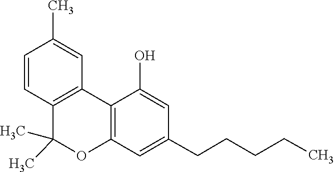 [SMILES: CCCCCc1cc(O)c2c(c1)OC(C)(C)c1ccc(C)cc1-2] | Likely degradation product of THC m/z 311.2006 |
| cannabinolic acid (CBNA) | 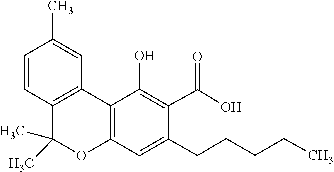 [SMILES: CCCCCc1cc2c(c(O)c1C(=O)O)-c1cc(C)ccc1C(C)(C)O2] | m/z 355.1904 |
| tetrahydrocannabivarin (THCV) | 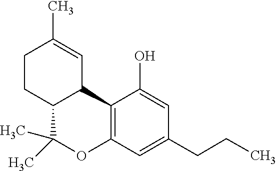 | decarboxylation product of THCVA m/z 287.2006 |
| tetrahydrocannabivarinic acid (THCVA) | 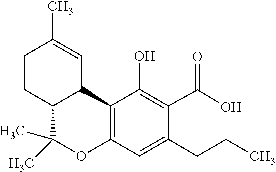 | m/z 331.1904 |
| cannabidivarin (CBDV) | 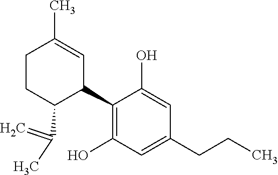 | m/z 287.2006 |
| cannabidivarinic acid (CBDVA) | 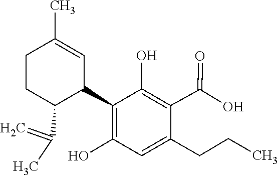 | m/z 331.1904 |
| Δ8-tetrahydrocannabinol (d8-THC) | 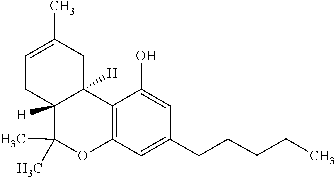 [SMILES: CCCCCc1cc(O)c2c(c1)OC(C)(C)[C@@H]1CC=C(C)C[C@@H]21] | m/z 315.2319 |

Cannabinoids are synthesised in cannabis plants as carboxylic acids. While some decarboxylation may occur in the plant, decarboxylation typically occurs post-harvest and is increased by exposing plant material to heat (Sanchez and Verpoote, 2008, Plant Cell Physiol, 49(12): 1767-82). Decarboxylation is usually achieved by drying and/or heating the plant material. Persons skilled in the art would be familiar with methods by which decarboxylation of cannabinoids can be promoted, illustrative examples of which include air-drying, combustion, vaporisation, curing, heating and baking.

### Cannabinoid Profile

The term “cannabinoid profile” refers to a representation of the type, amount, level, ratio and/or proportion of cannabinoids that are present in the cannabis plant or part thereof, as typically measured within plant material derived from the plant or plant part, including an extract therefrom.

The term “enriched” is used herein to refer to a selectively higher level of one or more cannabinoids in the cannabis plant or part thereof. For example, a cannabinoid profile enriched for total CBD refers to plant material in which the amount of total CBD (total CBD and/or total CBDA) is greater than the amount of any of the other cannabinoids that may also be present (including constitutively present) in the plant material.

The cannabinoid profile in a cannabis plant will typically predominantly comprise the acidic form of the cannabinoids, but may also comprise some decarboxylated (neutral) forms thereof, at various concentrations or levels at any given time (i.e., at propagation, growth, harvest, drying, curing, etc.). Thus, the term “total cannabinoid” is used herein to refer to the decarboxylated and/or acid form of said cannabinoid. For example, “total CBD” refers to total CBD and/or total CBDA, “total THC” refers to total THC and/or total THCA, “total CBC” refers to CBC and/or CBCA, “total CBG” refers to CBG and/or CBGA, “total CBN” refers to total CBN and/or total CBNA, “total THCV” refers to total THCV and/or total THCVA, “total CBDV” refers to total CBDV and/or total CBDVA, and so forth.

“Cannabidiolic acid” or “CBDA” is a derivative of cannabigerolic acid (CBGA), which is converted to CBDA by CBDA synthase. Its neutral form, “cannabidiol” or “CBD” has antagonist activity on agonists of the CB1 and CB2 receptors. CBD has also been shown to act as an antagonist of the putative cannabinoid receptor, GPR55. CBD is commonly associated with therapeutic or medicinal effects of cannabis and has been suggested for use as a sedative, anti-inflammatory, anti-anxiety, anti-nausea, atypical anti-psychotic, and as a cancer treatment. CBD can also increase alertness, and attenuate the memory impairing effect of THC.

The female cannabis plant described herein produces inflorescence comprising a cannabinoid profile that is characterised by an approximately equal level of total CBD and THC in the plant material, which is greater than the level of other minor cannabinoids. Accordingly, the cannabis plant of the invention may be variously described as “high-CBD and -THC”, “CBD- and THC-enriched” or “high-CBD and -THC”. Those skilled in the art would understand this terminology to mean a cannabis plant that produced higher levels of CBD and/or CBDA and THC and/or THCA, relative to the level of other minor cannabinoids.

In an embodiment, the level of total CBD is at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material.

“Δ-9-tetrahydrocannabinolic acid” or “THCA” is also synthesised from the CBGA precursor by THCA synthase. The neutral form “Δ-9-tetrahydrocannabinol” is associated with psychoactive effects of cannabis, which are primarily mediated by its activation of CB1G-protein coupled receptors, which result in a decrease in the concentration of cyclic AMP (cAMP) through the inhibition of adenylate cyclase. THC also exhibits partial agonist activity at the cannabinoid receptors CB1 and CB2. CB1 is mainly associated with the central nervous system, while CB2 is expressed predominantly in the cells of the immune system. As a result, THC is also associated with pain relief, relaxation, fatigue, appetite stimulation, and alteration of the visual, auditory and olfactory senses. Furthermore, more recent studies have indicated that THC mediates an anti-cholinesterase action, which may suggest its use for the treatment of Alzheimer's disease and myasthenia (Eubanks et al., 2006, Molecular Pharmaceuticals, 3(6): 773-7).

In an embodiment, the level of total THC is at least 20%, preferably at least 21%, preferably at least 22%, preferably at least 23%, preferably at least 24%, preferably at least 25%, preferably at least 26%, preferably at least 27%, preferably at least 28%, preferably at least 29%, preferably at least 30%, preferably at least 31%, preferably at least 32%, preferably at least 33%, preferably at least 34%, preferably at least 35%, preferably at least 36%, preferably at least 37%, preferably at least 38%, preferably at least 39%, preferably at least 40%, preferably at least 41%, preferably at least 42%, preferably at least 43%, preferably at least 44%, preferably at least 45%, preferably at least 46%, preferably at least 47%, preferably at least 48% or more preferably at least 49% by weight of the total cannabinoid content of the dry weight of plant material.

In an embodiment, total CBD and total THC are present in a ratio of from about 1:1 to about 5:1, preferably from about 1:1 to about 4:1, or more preferably from about 1:1 to about 3:1 (CBD:THC). In another embodiment, total CBD and total THC are present in a ratio of about 1:1.

In an embodiment, the reference cannabinoid is total CBC. In another embodiment, total CBD and total THC (CBD+THC) is present at a ratio of from about 10:1 to about 50:1 to the level of total CBC, preferably from about 10:1 to about 49:1, preferably from about 10:1 to about 48:1, preferably from about 10:1 to about 47:1, preferably from about 10:1 to about 46:1, preferably from about 10:1 to about 45:1, preferably from about 10:1 to about 44:1, preferably from about 10:1 to about 43:1, preferably from about 10:1 to about 42:1, preferably from about 10:1 to about 41:1, or more preferably from about 10:1 to about 40:1 (CBD+THC:CBC).

In another embodiment, the level of total CBC is from about 1% to about 10%, preferably from about 1% to about 9%, preferably from about 1% to about 8%, preferably from about 1% to about 7%, preferably from about 1% to about 6%, preferably from about 1% to about 5%, preferably from about 2% to about 10%, preferably from about 2% to about 9%, preferably from about 2% to about 8%, preferably from about 2% to about 7%, preferably from about 2% to about 6%, or more preferably from about 2% to about 5% by weight of the total cannabinoid content of the dry weight of plant material.

In an embodiment, the reference cannabinoid is total CBG. In another embodiment, CBD+THC is present at a ratio of from about 10:1 to about 110:1 to the level of total CBG, preferably from about 20:1 to about 110:1, preferably from about 10:1 to about 110:1, preferably from about 30:1 to about 110:1, preferably from about 40:1 to about 110:1, preferably from about 50:1 to about 110:1, preferably from about 60:1 to about 110:1, preferably from about 70:1 to about 110:1, preferably from about 80:1 to about 110:1, preferably from about 90:1 to about 110:1, or more preferably from about 100:1 to about 110:1 (CBD+THC:CBG).

In another embodiment, the level of total CBG is from about 0.5% to about 10%, preferably from about 0.5% to about 9%, preferably from about 0.5% to about 8%, preferably from about 0.5% to about 7%, preferably from about 0.5% to about 6%, or more preferably from about 0.5% to about 5% by weight of the total cannabinoid content of the dry weight of plant material.

In an embodiment, the reference cannabinoid is total CBN. In another embodiment, CBD+THC is present at a ratio of from about 400:1 to about 4000:1 to the level of total CBN, preferably from about 400:1 to about 3900:1, preferably from about 400:1 to about 3800:1, preferably from about 400:1 to about 3700:1, preferably from about 400:1 to about 3600:1, preferably from about 400:1 to about 3500:1, preferably from about 400:1 to about 3400:1, preferably from about 400:1 to about 3300:1, preferably from about 400:1 to about 3200:1, preferably from about 400:1 to about 3100:1, or more preferably from about 400:1 to about 3000:1 (CBD+THC:CBG).

In another embodiment, the level of total CBN is from about 0.01% to about 1%, preferably from about 0.01% to about 0.9%, preferably from about 0.01% to about 0.8%, preferably from about 0.01% to about 0.7%, preferably from about 0.01% to about 0.6%, or more preferably from about 0.01% to about 0.5% by weight of the total cannabinoid content of the dry weight of plant material.

In an embodiment, the reference cannabinoid is total CBDV. In another embodiment, CBD+THC is present at a ratio of from about 100:1 to about 2000:1 to the level of total CBDV, preferably from about 100:1 to about 1900:1, preferably from about 100:1 to about 1800:1, preferably from about 100:1 to about 1700:1, preferably from about 100:1 to about 1600:1, preferably from about 100:1 to about 1500:1, preferably from about 100:1 to about 1400:1, preferably from about 100:1 to about 1300:1, preferably from about 100:1 to about 1200:1, preferably from about 100:1 to about 1100:1, or more preferably from about 100:1 to about 1000:1 (CBD+THC:CBDV).

In another embodiment, the level of total CBDV is from about 0.01% to about 1%, preferably from about 0.02% to about 1%, preferably from about 0.03% to about 1%, preferably from about 0.04% to about 1%, or more preferably from about 0.05% to about 1% by weight of the total cannabinoid content of the of dry weight of plant material.

In an embodiment, the reference cannabinoid is total THCV. In another embodiment, CBD+THC is present at a ratio of from about 100:1 to about 600:1 to the level of total THCV, preferably from about 100:1 to about 590:1, preferably from about 100:1 to about 580:1, preferably from about 100:1 to about 570:1, preferably from about 100:1 to about 560:1, preferably from about 100:1 to about 550:1, preferably from about 100:1 to about 540:1, preferably from about 100:1 to about 530:1, preferably from about 100:1 to about 520:1, preferably from about 100:1 to about 510:1, or more preferably from about 100:1 to about 500:1 (CBD+THC:THCV).

In another embodiment, the level of total THCV is from about 0.01% to about 1%, preferably from about 0.02% to about 1%, preferably from about 0.03% to about 0.1%, preferably from about 0.04% to about 1%, preferably from about 0.05% to about 1%, preferably from about 0.06% to about 1%, preferably from about 0.07% to about 1%, preferably from about 0.08% to about 1%, preferably from about 0.09% to about 1%, or more preferably from about 0.1% to about 1% by weight of the total cannabinoid content of the dry weight of plant material.

### Terpenes

The term “terpene” as used herein, refers to a class of organic hydrocarbon compounds, which are produced by a variety of plants. Cannabis plants produce and accumulate different terpenes, such as monoterpenes and sesquiterpenes, in the glandular trichomes of the female inflorescence. The term “terpene” includes “terpenoids” or “isoprenoids”, which are modified terpenes that contain additional functional groups.

Terpenes are responsible for much of the scent of cannabis flowers and contribute to the unique flavour qualities of cannabis products. Terpenes will be known to persons skilled in the art, illustrative examples of which are provided in Table 2. Table 2. Terpenes and their properties

TABLE 2
Terpenes and their properties.

| | | Mass/Charge number |
|---|---|---|
| Name | Structure | (m/z)* |
| α-Phellandrene | 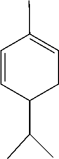 | m/z 93.0 |
| α-Pinene (+/-) | 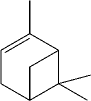 | m/z 93.0 |
| Camphene | 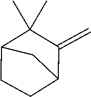 | m/z 93.0 |
| β-Pinene (+/-) | 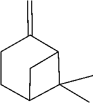 | m/z 93.0 |
| Myrcene | 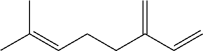 | m/z 93.0 |
| Limonene | 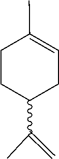 | m/z 68.1 |
| 3-Carene | 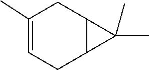 | |
| Eucalyptol | 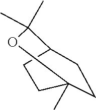 | m/z 81.0 |
| γ-Terpinene | 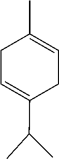 | m/z 93.1 |
| Linalool | 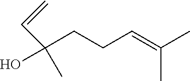 | m/z 93.0 |
| γ-Elemene | 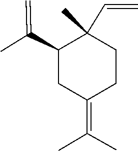 | m/z 121.0 |
| Humulene | 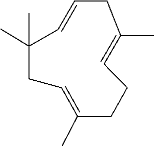 | m/z 93.0 |
| Nerolidol | 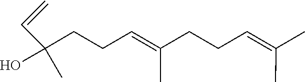 [SMILES: C=CC(C)(O)CC/C=C(\C)CCC=C(C)C] | m/z 222.4 |
| Guaia-3,9-diene | 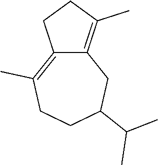 | m/z 161.1 |
| Caryophyllene | 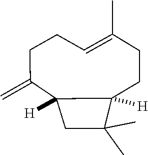 | m/z 69.2 |

*The molecular ion is not necessarily seen for all compounds

Terpene biosynthesis in plants typically involves two pathways to produce the general 5-carbon isoprenoid diphosphate precursors of all terpenes: the plastidial methylerythritol phosphate (MEP) pathway and the cytosolic mevalonate (MEV) pathway. These pathways control the different substrate pools available for terpene synthases (TPS).

The term “trichomes” as used herein refers to epidermal structures present on the floral buds of the female cannabis plant, as well as the surrounding leaves and most aerial parts of the plant. Cannabis exhibits both glandular and non-glandular trichomes, which may be distinguished based on their secretion ability and morphology. In particular, it is the glandular trichomes that comprise secretory cells that are specialized structures that synthesize high amounts of secondary metabolites, such as the phytocannabinoids, terpenes, and phenolics described above. However, other parts of the plant, such as seeds, roots and pollen are also capable of producing low levels of phytocannabinoids.

### Terpene Profile

The term “terpene profile” as used herein refers to a representation of the type, amount, level, ratio and/or proportion of terpenes that are present in a female cannabis plant or part thereof, as typically measured within plant material derived from the plant or plant part, including an extract therefrom.

The terpene profile in a female cannabis plant will be determined based on genetic, environmental and developmental factors, therefore particular terpenes may be present at various amounts, levels, ratios and/or proportions at any given time (i.e., at propagation, growth, harvest, drying, curing, etc.).

In an embodiment, the terpene profile comprises monoterpenes and sesquiterpenes.

Monoterpenes consist of two isoprene units and may be liner or contain ring structures. The primary function of monoterpenes is to protect plants from infection by fungal and bacterial pathogens and insect pests. Monoterpenes would be known to persons skilled in the art, illustrative embodiments of which include α-phellandrene, α-pinene, camphene, β-pinene, myrcene, limonene, eucalyptol, γ-terpinene and linalool.

Sesquiterpenes differ from other common terpenes as they contain one additional isoprene unit, which creates a 15 carbon structure. The primary function of sesquiterpenes is as a pheromone for the bud and flower. Sesquiterpenes would be known to persons skilled in the art, illustrative embodiments of which include γ-elemene, humulene, nerolidol, guaia-3,9-diene and caryophyllene.

In an embodiment, the female cannabis plant produces inflorescence comprising a terpene profile that comprises a level of monoterpenes that correlates with the level of total THC. In a preferred embodiment, the terpene profile comprises a high level of monoterpenes that correlates to a high level of total THC. In another embodiment, the terpene profile comprises a level of sesquiterpenes that correlates with the level of total CBD. In a preferred embodiment, the terpene profile comprises a high level of sesquiterpenes that correlates with a high level of total CBD.

In an embodiment, the female cannabis plant produces inflorescence comprising a terpene profile comprising terpenes selected from the group consisting of α-phellandrene, α-pinene, camphene, β-pinene, myrcene, limonene, eucalyptol, γ-terpinene, linalool, γ-elemene, humulene, nerolidol, guaia-3,9-diene and caryophyllene. In a preferred embodiment, the female cannabis plant produces inflorescence comprising a terpene profile comprising terpenes selected from the group consisting of myrcene and β-pinene.

“Myrcene” is a monoterpinoid derivative of β-pinene. Myrcene has been associated with the therapeutic or medicinal effects of cannabis and has been suggested for use as a sedative, hypnotic, analgesic and muscle relaxant. Myrcene is also hypothesised to attenuate the activity of other cannabinoids and terpenes as part of the “entourage effect” as described in, for example, Russo, 2011, British Journal of Pharmacology, 163(7): 1344-1364.

“β-pinene” is a monoterpene that is characterised by a woody-green, pine-like smell. β-pinene has been shown to act as a topical antiseptic and a bronchodilator. β-pinene is also capable of crossing the blood-brain barrier and it is hypothesised that β-pinene inhibits the influence of THC as part of the entourage effect, as described elsewhere herein.

In an embodiment, the level of myrcene is present at a ratio of from about 100:1 to about 1:1 to the level of β-pinene. The range “from about 100:1 to about 1:1” includes, for example, 100:1, 99:1, 98:1, 97:1, 96:1, 95:1, 94:1, 93:1, 92:1, 91:1, 90:1, 89:1, 88:1, 87:1, 86:1, 85:1, 84:1, 83:1, 82:1, 81:1, 80:1, 79:1, 78:1, 77:1, 76:1, 75:1, 74:1, 73:1, 72:1, 71:1, 70:1, 69:1, 68:1, 67:1, 66:1, 65:1, 64:1, 63:1, 62:1, 61:1, 60:1, 59:1, 58:1, 57:1, 56:1, 55:1, 54:1, 53:1, 52:1, 51:1, 50:1, 49:1, 48:1, 47:1, 46:1, 45:1, 44:1, 43:1, 42:1, 41:1, 40:1, 39:1, 38:1, 37:1, 36:1, 35:1, 34:1, 33:1, 32:1, 31:1, 30:1, 29:1, 28:1, 27:1, 26:1, 25:1, 24:1, 23:1, 22:1, 21:1, 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1 and 1:1. Thus, in an embodiment, the ratio of the level of myrcene to the level of β-pinene is about preferably about 100:1, preferably about 99:1, preferably about 98:1, preferably about 97:1, preferably about 96:1, preferably about 95:1, preferably about 94:1, preferably about 93:1, preferably about 92:1, preferably about 91:1, preferably about 90:1, preferably about 89:1, preferably about 88:1, preferably about 87:1, preferably about 86:1, preferably about 85:1, preferably about 84:1, preferably about 83:1, preferably about 82:1, preferably about 81:1, preferably about 80:1, preferably about 79:1, preferably about 78:1, preferably about 77:1, preferably about 76:1, preferably about 75:1, preferably about 74:1, preferably about 73:1, preferably about 72:1, preferably about 71:1, preferably about 70:1, preferably about 69:1, preferably about 68:1, preferably about 67:1, preferably about 66:1, preferably about 65:1, preferably about 64:1, preferably about 63:1, preferably about 62:1, preferably about 61:1, preferably about 60:1, preferably about 59:1, preferably about 58:1, preferably about 57:1, preferably about 56:1, preferably about 55:1, preferably about 54:1, preferably about 53:1, preferably about 52:1, preferably about 51:1, preferably about 50:1, preferably about 49:1, preferably about 48:1, preferably about 47:1, preferably about 46:1, preferably about 45:1, preferably about 44:1, preferably about 43:1, preferably about 42:1, preferably about 41:1, preferably about 40:1, preferably about 39:1, preferably about 38:1, preferably about 37:1, preferably about 36:1, preferably about 35:1, preferably about 34:1, preferably about 33:1, preferably about 32:1, preferably about 31:1, preferably about 30:1, preferably about 29:1, preferably about 28:1, preferably about 27:1, preferably about 26:1, preferably about 25:1, preferably about 24:1, preferably about 23:1, preferably about 22:1, preferably about 21:1, preferably about 20:1, preferably about 19:1, preferably about 18:1, preferably about 17:1, preferably about 16:1, preferably about 15:1, preferably about 14:1, preferably about 13:1, preferably about 12:1, preferably about 11:1, preferably about 10:1, preferably about 9:1, preferably about 8:1, preferably about 7:1, preferably about 6:1, preferably about 5:1, preferably about 4:1, preferably about 3:1, preferably about 2:1, or more preferably about 1:1.

In an embodiment, the level of myrcene is present at a ratio of from about 40:1 to about 4:1 to the level of β-pinene.

### Method for Determining the Sex of a Cannabis Plant

Cannabis plant sex determination is considered to be important during production of cannabis to ensure that male cannabis plants are identified before pollen dispersion. Early identification of male cannabis plants ensures that such plants are eliminated from the crop before male reproductive tissues mature and pollination occurs.

The sex of a cannabis plant is typically determined by morphological evaluation of floral tissue. However, anomalies in flower development, such as the appearance of hermaphrodite flowers or the development of mixed flowers (i.e., bearing both male and female flowers), or the total or partial reversion of sex can make it difficult to identify female or male cannabis plants from morphological evaluation alone.

The methods disclosed herein may suitably be used to identify female or male cannabis plants from a plurality of cannabis plants comprising cannabis plants of undetermined sex, for example, early in the flower bud maturation cycle (i.e., Stage 1). This advantageously allows breeders, cultivators and the like to monitor their crop for male or hermaphroditic plants and, where necessary, remove and/or discard male cannabis plants before pollination occurs to produce a crop enriched for female cannabis plants.

Accordingly, in an aspect disclosed herein, there is provided a method for determining the sex of a cannabis plant, the method comprising:
a. providing a nucleic acid sample obtained from cannabis plant tissue;
b. determining the level of expression of one or more Cannabis sativa genes, or homologs thereof, wherein the one or more genes encode a gene product selected from the group consisting of:
   i. lipoxygenase,
   ii. cannabinoid synthesis protein,
   iii. geranyl diphosphate pathway protein,
   iv. MEP pathway protein,
   v. terpene synthesis protein,
   vi. MADs box floral initiation transcription factors,
   vii. cannabis allergens, and
   viii. leucine-rich repeat (LRR) containing protein;
c. comparing the level of expression of the one or more genes determined in (b) with a sex determination reference value; and
d. determining the sex of the cannabis plant based on the comparison made in (c).

The term “nucleic acid sample” as used herein refers to any “polynucleotide”, “polynucleotide sequence”, “nucleotide sequence”, “nucleic acid” or “nucleic acid sequence” comprising ribonucleic acid (RNA), messenger RNA (mRNA), complementary RNA (cRNA), deoxyribonucleic acid (DNA) or complementary DNA (cDNA).

In an embodiment, the nucleic acid sample comprises RNA.

The term “cannabis plant tissue” as used herein is to be understood to mean any part of the cannabis plant, including the leaves, stems, roots, and inflorescence, or parts thereof, as described elsewhere herein, illustrative examples of which include trichomes and glands.

In an embodiment, the cannabis plant tissue is selected from the group consisting of inflorescence, shoot, leaf, and root.

In an embodiment, the cannabis plant tissue is inflorescence.

The term “inflorescence” as used herein means the complete flower head of the cannabis plant, comprising stems, stalks, bracts, flowers and trichomes (i.e., glandular, sessile and stalked trichomes).

Male inflorescence consists of a perianth of five sepals that encloses the androecium, composed of five stamens bored by subtle stalks. The anthers at maturity undergo dehiscence longitudinally, releasing the pollen grains that are mostly wind dispersed.

Female inflorescence is composed by a green bract that completely wraps the rudimental perianth and the ovary. This latter is an uniloculate and has a short style that distally differentiates a bifid stigma.

In an embodiment, the cannabis plant tissue is developmental Stage 1 inflorescence.

In an embodiment, the sex determination reference value is representative of a level of expression of the one or more genes encoding gene products (i)-(viii) in cannabis plant tissue of a male cannabis plant or a plurality of male cannabis plants.

In an embodiment, a level of expression of one or more genes encoding gene products (i)-(vi) that exceeds the sex determination reference value is indicative that the cannabis plant is a female cannabis plant. In another embodiment, a level of expression of one or more genes encoding gene products (vii)-(viii) that is equal to or less than the sex determination reference value is indicative that the cannabis plant is a female cannabis plant.

In an embodiment, the cannabis allergen is selected from the group consisting of Betv1-like protein, pollen allergen, yes allergen, V5 allergen, and Par allergen.

In an embodiment, the cannabinoid synthesis protein is selected from the group consisting of THCA synthase, cannabidiolic synthase, olivetolic acid cyclase, polyketide synthase, chalcone synthase and 2-acylpholoroglucinol 4-prenyltansferase.

In an embodiment, the MEP pathway protein is selected from the group consisting of deoxyxyluose-5-phosphate synthase, 4-hydroxy-3-methylbut-2-en-1-yl diphosphate synthase, HDS, HDR, 4-hydroxy-3-methylbut-2-enyl diphosphate reductase, C-methyl-D-erythritol 2,4-cyclodiphosphate synthase, fatty acid desaturase, FAD2 and omega-6 fatty acid desaturase.

In an embodiment, the terpene synthesis protein is selected from the group consisting of terpene synthase, terpene cyclase/mutase, (−)-limonene synthase, (+)-alpha-pinene synthase, 3,5,7-trioxododecanoyl-CoA synthase, lupeol synthase, secologanin synthase and vinorine synthase.

In another aspect disclosed herein, there is provided a method for determining the sex of a cannabis plant, the method comprising:
a. providing a nucleic acid sample obtained from cannabis plant tissue;
b. determining the level of expression of one or more Cannabis sativa genes, or homologs thereof, wherein the one or more genes encode a gene product selected from the group consisting of:
   i. glycoside hydrolase,
   ii. naringenin-chalcone synthase,
   iii. lipoxygenase,
   iv. sieve-element inclusion protein,
   v. cannabis allergens,
   vi. leucine-rich repeat (LRR) containing protein,
   vii. F-box domain containing protein,
   viii. pseudo-autosomal region (PAR) containing protein, and
   ix. endonucleases;
c. comparing the level of expression of the one or more genes determined in (b) with a sex determination reference value; and
d. determining the sex of the cannabis plant based on the comparison made in (c).

In another aspect disclosed herein, there is provided a method for determining the sex of a cannabis plant, the method comprising:
a. providing a nucleic acid sample obtained from cannabis plant tissue;
b. determining the level of expression of one or more Cannabis sativa genes, or homologs thereof, wherein the one or more genes encode a gene product listed in Table 11;
c. comparing the level of expression of the one or more genes determined in (b) with a sex determination reference value; and
d. determining the sex of the cannabis plant based on the comparison made in (c).

### Methods for Determining the Developmental Stage of a Female Cannabis Plant Inflorescence

The methods disclosed herein may suitably be used to determine the developmental stage of female cannabis plant inflorescence during the inflorescence maturation cycle. This advantageously allows breeders, cultivators and the like to monitor their crop to ensure that their plants are harvested at a developmental stage for optimal cannabinoid or terpene production.

Thus, in another aspect disclosed herein, there is provided a method for determining the developmental stage of a female cannabis plant inflorescence, the method comprising:
a. providing a nucleic sample obtained from female cannabis inflorescence or a part thereof;
b. determining the level of expression of one or more Cannabis sativa genes, or homologs thereof, wherein the one or more genes encode a gene product selected from the group consisting of:
   x. cannabinoid synthesis protein,
   xi. terpene synthesis protein,
   xii. MEP pathway protein,
   xiii. MEV pathway protein, and
   xiv. MADs box floral initiation transcription factor;
c. comparing the level of expression of the one or more genes determined in (b) with a developmental reference value; and
d. determining the developmental stage of the inflorescence based on the comparison made in (c).

The developmental stage of the cannabis plant is defined herein refers to the developmental stage of inflorescence after the induction of flowering. As described elsewhere herein, developmental Stage 1 (i.e., immature floral bud) is between 0 to 35 days after induction of flowering (e.g., 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 days after induction of flowering); developmental Stage 2 is between 36 to 42 days after the induction of flowering (e.g. 36, 37, 38, 39, 40, 41, or 42 days after induction of flowering); developmental Stage 3 is between 43 and 49 days after induction of flowering (e.g., 43, 44, 45, 46, 47, 48, 49 days after induction of flowering); and developmental Stage 4 (i.e., mature floral bud) is between 50 to 59 days after induction of flowering (e.g., 50, 51, 52, 53, 54, 55, 56, 57, 58, or 59 days after induction of flowering).

In an embodiment, the nucleic acid sample is RNA.

In an embodiment, the nucleic acid sample obtained from a part of the inflorescence selected from the group consisting of flower and trichome.

In an embodiment, the nucleic acid sample is obtained from trichome.

In an embodiment, the developmental reference value is representative of a level of expression of the one or more genes encoding gene products (i)-(v) in a female cannabis inflorescence at developmental Stage 1 or a plurality of female cannabis inflorescence at developmental Stage 1.

In an embodiment, a level of expression of the one or more genes encoding gene products (i)-(v) that exceeds the developmental reference value is indicative that the inflorescence is at developmental Stage 4. In another embodiment, a level of expression of the one or more genes encoding gene product (v) that is equal to or less than the developmental reference value is indicative that the inflorescence is at developmental Stage 4.

In an embodiment, the cannabinoid synthesis protein is selected from the group consisting of THCA synthase and polyketide synthase.

In an embodiment, the terpene synthesis protein is selected from the group consisting of terpene syclase, terpene synthase, (−)-limonene synthase, (+)-alpha-pinene synthase, lupeol synthase, vinorine synthase and germacrene-A synthase.

In an embodiment, the MEP pathway protein is selected from the group consisting of HDR, fatty acid desaturase, delta-12 fatty acid desaturase, omega-6 fatty acid desaturase, delta-12-acyl-lipid desaturase, delta-12-oleic acid desaturase, delta-12 desaturase, delta-12-olate desaturase and delta-12-acyl-lipid desaturase.

In an embodiment, the MEV pathway protein is selected from the group consisting of 3-hydroxy-3-methylglutaryl coenzyme A reductase and 4-hydroxy-3-methylbut-2-enyl diphosphate reductase.

In another aspect disclosed herein, there is provided a method for determining the developmental stage of a female cannabis plant inflorescence, the method comprising:
a. providing a nucleic providing a nucleic sample obtained from female cannabis inflorescence or a part thereof;
b. determining the level of expression of one or more Cannabis sativa genes, or homologs thereof, wherein the one or more genes encode a gene product selected from the group consisting of:
   i. lipase,
   ii. sieve-element occlusion protein,
   iii. cytochrome P450,
   iv. fatty acid hydroylase,
   v. cytostolic mevalonate (MEV) pathway protein,
   vi. delta 15 desaturase,
   vii. delta 12 desaturase,
   viii. naringenin-chalcone synthase,
   ix. beta galactosidase,
   x. gibberellin 3-beta-dioxygenase,
   xi. chlorophyll A-B binding protein,
   xii. myrcene synthase,
   xiii. tryptophan aminotransferase-related protein 1 (TAR1),
   xiv. glycoside hydrolase,
   xv. terpene synthase, and
   xvi. plastidial methylerythritol phosphate (MEP) pathway protein;
c. comparing the level of expression of the one or more genes determined in (b) with a developmental reference value; and
d. determining the developmental stage of the inflorescence based on the comparison made in (c).

In another aspect disclosed herein, there is provided a method for determining the developmental stage of a female cannabis plant inflorescence, the method comprising:
a. providing a nucleic sample obtained from female cannabis inflorescence or a part thereof;
b. determining the level of expression of one or more Cannabis sativa genes, or homologs thereof, wherein the one or more genes encode a gene product selected from the gene products listed in Table 15.
c. comparing the level of expression of the one or more genes determined in (b) with a developmental reference value; and
d. determining the developmental stage of the inflorescence based on the comparison made in (c).

### Gene Expression Analysis

The present disclosure provides methods for determining a gene expression profile of cannabis plant tissue, such as female cannabis plant inflorescence or a part thereof. Methods for measuring gene expression would be known to persons skilled in the art, illustrative examples of which include serial analysis of gene expression (SAGE), microarrays, next generation sequencing (NGS) technology (i.e. RNA-Seq), real-time reverse transcriptase PCR (RT-qPCR), Northern blotting, quantitative PCR.

As described elsewhere herein, the sex of a cannabis plant may be determined by evaluating the level of expression of one or more Cannabis sativa genes, or homologs thereof, wherein the gene encodes one or more of the gene products selected from the group consisting of:
i. lipoxygenase,
ii. cannabinoid synthesis protein,
iii. geranyl diphosphate pathway protein,
iv. plastidial methylerythritol phosphate (MEP) pathway protein,
v. terpene synthesis protein,
vi. MADs box floral initiation transcription factor,
vii. cannabis allergens, and
viii. leucine-rich repeat (LRR) containing protein.

In another embodiment, the developmental stage of a female cannabis plant inflorescence may be determined by evaluating the level of expression of a Cannabis sativa gene or homolog thereof, wherein the gene encodes one or more of the gene products selected from the group consisting of:
i. cannabinoid synthesis protein,
ii. terpene synthesis protein,
iii. MEP pathway protein,
iv. MEV pathway protein, and
v. MADs box floral initiation transcription factor.

In yet another embodiment, a hypoallergenic cannabis plant may be selected by evaluating a level of expression of a Cannabis sativa gene, or homolog thereof, wherein the gene encodes a cannabis allergen.

The terms “level”, “content”, “concentration” and the like, are used interchangeably herein to describe the expression of the referenced Cannabis sativa gene or homolog thereof, and may be represented in absolute terms (e.g., mg/g, mg/ml, etc.) or in relative terms, such as a fold change and log-ratios thereof (e.g., log 2FoldChange, etc.).

In an embodiment, the level of gene expression is represented by fold change. In a preferred embodiment, the level of gene expression is represented by log 2FoldChange.

In an embodiment, the log 2FoldChange of the one or more Cannabis sativa genes, or homologs thereof, may be from about 1 to about 100. The range “from about 1 to about 100” includes, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100.

The term “expression” is used herein to denote a measurable presence of the referenced Cannabis sativa gene or homolog thereof.

The term “homolog” typically refers to a gene with similar biological activity, although differs in nucleotide sequence at one or more positions when the sequences are aligned. Generally, homologs will have at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a particular nucleotide sequence, as determined, for example, by sequence alignment programs known in the art using default parameters (e.g. BLASTn)

Homologs of Cannabis sativa genes may be found in the same species, in related species and/or sub-species, or in different species. For example, for a Cannabis sativa gene, homologs include those other plant species. Suitable plant species would be known to persons skilled in the art, illustrative examples of which include members of the Cannabaceae family (e.g., Trema, Parasponia, Humulus).

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The term “cannabinoid synthesis protein” as used herein refers to a family of proteins that are known to be involved in the biosynthesis of cannabinoids. Suitable cannabinoid synthesis proteins would be known to persons skilled in the art, illustrative examples of which include THCA synthase, cannabidiolic synthase, olivetolic acid cyclase, polyketide synthases, chalcone synthase and 2-acylpholoroglucinol 4-prenyltransferase.

In an embodiment, the cannabinoid synthesis protein is selected from the group consisting of THCA synthase, cannabidiolic synthase, olivetolic acid cyclase, polyketide synthases, chalcone synthase and 2-acylpholoroglucinol 4-prenyltransferase.

In another embodiment, the cannabinoid synthesis protein is selected from the group consisting of THCA synthase and polyketide synthases.

The term “terpene synthesis protein” as used herein refers to a family of proteins that are known to be involved in the biosynthesis of terpenes. Suitable terpene synthesis proteins would be known to persons skilled in the art, illustrative examples of which include terpene synthase, terpene cyclase/mutase, (−)-limonene synthase, (+)-alpha-pinene synthase, 3,5,7-trioxododecanoyl-CoA synthase, lupeol synthase, secologanin synthase, vinorine synthase and germacrene-A synthase.

In an embodiment, the terpene synthesis protein is selected from the group consisting of terpene synthase, terpene cyclase/mutase, (−)-limonene synthase, (+)-alpha-pinene synthase, 3,5,7-trioxododecanoyl-CoA synthase, lupeol synthase, secologanin synthase and vinorine synthase.

In another embodiment, the terpene synthesis protein is selected from the group consisting of terpene cyclase, terpene synthase, (−)-limonene synthase, (+)-alpha-pinene synthase, lupeol synthase, vinorine synthase and germacrene-A synthase.

The term “cannabis allergens” as used herein refer to proteins that are known to cause hypersensitivity or anaphylactic response. Suitable cannabis allergens would be known to persons skilled in the art, illustrative examples include RuBisCO, oxygen enhancer protein 2, lipid transfer protein (LTP) as detailed by Nayak et al. (Ann Allergy Asthma Immunol. 2013, 111(2013): 32-37).

In an embodiment, the cannabis allergens are selected from the group consisting of Betv1-like protein, glyceraldehyde-3-phosphate dehydrogenase, phosphoglycerate kinase, heat shock binding protein 70, ribulose-1,5-biphosphate carboxylase/oxygenase, non-specific lipid transfer protein (nt-LTP) and Light Oxygen Voltage (LOV) domain containing protein. In a preferred embodiment, the cannabis allergen is selected from the group consisting of Betv1-like protein, pollen allergen, yes allergen, V5 allergen, and Par allergen.

The terms “cytosolic mevalonate” or “MEV” pathway protein refers to the proteins that comprise a major terpene biosynthesis pathway described elsewhere herein. In an embodiment, the MEV pathway proteins are encoded by a Cannabis sativa gene selected from the group consisting of HGMS, HGMR1, HGMR2, CMK, PMK, IDI, FPPS1 and FPPS2.

In an embodiment, the MEV pathway protein is selected from the group consisting of 3-hydroxy-3-methylglutaryl coenzyme A reductase and 4-hydroxy-3-methylbut-2-enyl diphosphate reductase.

The terms “plastidial methylerythritol phosphate” or “MEP” pathway protein refers to the proteins that comprise a major terpene biosynthesis pathway described elsewhere herein. In an embodiment, the MEP pathway proteins are encoded by a Cannabis sativa gene selected from the group consisting of DXS1, DXS2, MCT, CMK, HDS, HDR and GPPS.

In an embodiment, the MEP pathway protein is selected from the group consisting of HDR, fatty acid desaturase, delta-12 fatty acid desaturase, omega-6 fatty acid desaturase, delta-12-acyl-lipid desaturase, delta-12-oleic acid desaturase, delta-12 desaturase, delta-12-olate desaturase and delta-12-acyl-lipid desaturase.

In another embodiment, the MEP pathway protein is selected from the group consisting of deoxyxyluose-5-phosphate synthase, 4-hydroxy-3-methylbut-2-en-1-yl diphosphate synthase, HDS, HDR, 4-hydroxy-3-methylbut-2-enyl diphosphate reductase, C-methyl-D-erythritol 2,4-cyclodiphosphate synthase, fatty acid desaturase, FAD2 and omega-6 fatty acid desaturase.

The term “geranyl diphosphate pathway proteins” refers to the proteins that having aromatic prenyltransferase activity, which have been previously associated with cannabinoid biosynthesis in Cannabis sativa (see, e.g., WO 2011/017798).

The terms “terpene synthase” or “TPS” may be used interchangeably herein to refer to a family or proteins that synthesise terpenes. In an embodiment, the terpene synthase is encoded by a Cannabis sativa gene selected from the group consisting of TPS1, TPS2, TPS3, TPS6, TPS7, TPS8, TPS9, TPS11 and TPS12.

The term “MADs box floral initiation transcription factors” as used herein refers to a family of proteins (i.e., transcription factors) that are known to control gene expression and identity of floral organs during plant development, as described, for example, by Theiben et al. (2016, Development, 143: 3259-3271).

### Reference Values

The methods disclosed herein suitably comprise a comparative step in which the level of expression of the one or more Cannabis sativa genes or homologs thereof is compared to a reference value.

The term “reference value” as used herein typically refers to a level of expression of one or more Cannabis sativa genes or homologs thereof representative of the level of expression of the one or more Cannabis sativa genes or homologs thereof in particular cohort or population of cannabis plants (i.e., male cannabis plants, female cannabis plants). In an illustrative example, the comparison may be carried out using a reference value that is representative of a known or predetermined level of expression of the defined Cannabis sativa gene or homolog thereof in female cannabis inflorescence a specified developmental stage.

The reference value may be represented as an absolute number, or as a mean value (e.g., mean+/−standard deviation, such as when the reference value is derived from (i.e., representative of) a population of cannabis plants. The reference value may be equal to or not significantly different from the level of expression of the one or more Cannabis sativa genes or homologs thereof in a sample population representative of male cannabis plants, female cannabis plants and female cannabis plants at a particular developmental stage.

Whilst persons skilled in the art would understand that using a reference value that is derived from a sample population of cannabis plants is likely to provide a more accurate representation of the level of expression in that particular population (e.g., for the purposes of the methods disclosed herein), in some embodiments, the reference value can be a level of expression of the one or more Cannabis sativa genes or homologs thereof in a single male cannabis plant or female cannabis plant. In other embodiments, the reference value can be a level of expression of the one or more Cannabis sativa genes or homologs thereof in a single female cannabis inflorescence at a defined developmental stage.

In an embodiment, the “sex determination reference value” refers to the level of expression of the one or more Cannabis sativa genes or homologs thereof in the cannabis plant tissue of a female cannabis plant.

In an embodiment, the “sex determination reference value” refers to the level of expression of the one or more Cannabis sativa genes, or homologs thereof, in the cannabis plant tissue of a male cannabis plant.

As described elsewhere herein, in an embodiment, a level of expression of the one or more genes encoding gene products (i)-(vi) that exceeds the sex determination reference value is indicative that the cannabis plant is a female cannabis plant.

In a preferred embodiment, a level of expression of the one or more genes encoding gene products (i)-(vi) that exceeds the sex determination reference value is indicative that the cannabis plant is a female cannabis plant, wherein the sex determination reference value is representative of a level of expression of the one or more genes encoding gene products (i)-(vi) in cannabis plant tissue of a male cannabis plant or plurality of male cannabis plants.

In another embodiment, a level of expression of the one or more genes encoding gene products (vii)-(viii) that is equal to or less than the sex determination reference value is indicative that the cannabis plant is a female cannabis plant. In a preferred embodiment, a level of expression of the one or more genes encoding gene products (vii)-(viii) that exceeds the sex determination reference value is indicative that the cannabis plant is a female cannabis plant, wherein the sex determination reference value is representative of a level of expression of the one or more genes encoding gene products (vii)-(viii) in cannabis plant tissue of a male cannabis plant or plurality of male cannabis plants.

In an embodiment, the “developmental reference value” refers to the level of expression of the one or more Cannabis sativa genes, or homologs thereof, in female cannabis inflorescence at developmental Stage 1 or a plurality of female cannabis inflorescence at developmental Stage 1.

In an embodiment, the “developmental reference value” refers to the level of expression of the one or more Cannabis sativa genes, or homologs thereof, in female cannabis inflorescence at developmental Stage 2 or a plurality of female cannabis inflorescence at developmental Stage 2.

In an embodiment, the “developmental reference value” refers to the level of expression of the one or more Cannabis sativa genes, or homologs thereof, in female cannabis inflorescence at developmental Stage 3 or a plurality of female cannabis inflorescence at developmental Stage 3.

In an embodiment, the “developmental reference value” refers to the level of expression of the one or more Cannabis sativa genes, or homologs thereof, in female cannabis inflorescence at developmental Stage 4 or a plurality of female cannabis inflorescence at developmental Stage 4.

As described elsewhere herein, in a preferred embodiment, a level of expression of the one or more genes encoding gene products (i)-(iv) that exceeds the developmental reference value is indicative that the inflorescence is at developmental Stage 4, wherein developmental reference value is representative of a level of expression of the one or more genes encoding gene products (i)-(iv) in a female cannabis inflorescence at developmental Stage 1 or a plurality of female cannabis inflorescence at developmental Stage 1.

In another preferred embodiment, a level of expression of the one or more genes encoding gene products (v) that exceeds the developmental reference value is indicative that the inflorescence is at developmental Stage 4, wherein developmental reference value is representative of a level of expression of the one or more genes encoding gene products (v) in a female cannabis inflorescence at developmental Stage 1 or a plurality of female cannabis inflorescence at developmental Stage 1.

In an embodiment, the “allergen reference value” refers to the level of expression of the one or more Cannabis sativa genes, or homologs thereof, in the cannabis plant tissue of a female cannabis plant.

In an embodiment, a level of expression of the one or more genes encoding a cannabis allergen that is less than the allergen reference value is indicative that the cannabis plant is a hypoallergenic cannabis plant.

### Methods for Monitoring the Development of a Female Cannabis Plant

The methods disclosed herein may suitably be used to monitor changes to the developmental status of female cannabis plants, for example, during the flower bud maturation cycle. This advantageously allows breeders, cultivators and the like to monitor their crop to ensure that their plants are harvested at a developmental stage for optimal resin production.

Thus, in another aspect disclosed herein, there is provided a method for monitoring the development of female cannabis plant inflorescence, the method comprising:
a. determining the developmental stage of a first inflorescence from a female cannabis plant in accordance with the methods disclosed herein;
b. determining the development stage of a second inflorescence from the plant of (a) in accordance with the methods disclosed herein at a subsequent time point in the growth cycle of the plant; and
c. comparing the developmental stage determined at (a) and (b) to evaluate whether there has been a change in the developmental stage of the inflorescence.

In another disclosed herein, there is provided a method for standardising the harvesting of female cannabis plants, the method comprising:
a. determining the developmental stage of an inflorescence from a female cannabis plant in accordance with the methods disclosed herein;
b. determining the developmental stage of an inflorescence from one or more additional female cannabis plants in accordance with the methods disclosed herein;
c. comparing the developmental stage determined at (a) and (b) to evaluate if the female cannabis plants have inflorescence at the same developmental stage;
d. optionally, determining the developmental stage of additional inflorescence from the plants of (a) and (b) in accordance with the methods disclosed herein at a subsequent time point in the growth cycle of the plants; and
e. harvesting the plants when the inflorescence are determined to be at the same developmental stage.

### Methods for Selecting Cannabis Plants

In yet another aspect disclosed herein, there is provided a method for selecting a female cannabis plant for harvest, wherein the female cannabis plant produces inflorescence comprising a cannabinoid profile enriched for total CBD and total THC, the method comprising:
a. determining the developmental stage of an inflorescence from a female cannabis plant in accordance with the methods disclosed herein;
b. optionally, determining the developmental stage of an additional inflorescence from the plant of (a) in accordance with the methods disclosed herein at a subsequent time point in the growth cycle of the plant;
c. harvesting the plant when the inflorescence are determined to be at developmental Stage 4,
   wherein the cannabinoid profile comprises a level of total CBD and a level of total THC at a ratio of from about 1:1 to about 5:1 (CBD:THC), wherein the total CDB comprises cannabidiol (CBD) and/or cannabidiolic acid (CBDA), and wherein the total THC comprises Δ-9-tetrahydrocannabinol (THC) and/or Δ-9-tetrahydrocannabinolic acid (THCA), and wherein the level of total CBD and total THC (CBD+THC) is greater than the level of a reference cannabinoid selected from the group consisting of:
d. total CBC, wherein total CBC comprises cannabichromene (CBC) and/or cannabichromene acid (CBCA), and wherein CBD+THC is present at a ratio of from about 10:1 to about 50:1 to the level of total CBC (CBD+THC:CBC);
e. total CBG, wherein the total CBG comprises cannabigerol (CBG) and/or cannabigerolic acid (CBGA), and wherein CBD+THC is present at a ratio of from about 10:1 to about 110:1 to the level of total CBG (CBD+THC:CBG);
f. total CBN, wherein the total CBN comprises cannabinol (CBN) and/or cannabinolic acid (CBNA), and wherein CBD+THC is present at a ratio of from about 400:1 to about 4000:1 to the level of total CBN (CBD+THC:CBN);
g. total THCV, wherein the total THCV comprises tetrahydrocannabivarin (THCV) and/or tetrahydrocannabivarinic acid (THCVA), and wherein CBD+THC is present at a ratio of from about 100:1 to about 600:1 to the level of total THCV (CBD+THC:THCV); and
h. total CBDV, wherein the total CBDV comprises cannabidivarin (CBDV) and/or cannabidivarinic acid (CBDVA), and wherein CBD+THC is present at a ratio of from about 100:1 to about 2000:1 to the level of CBDV (CBD+THC:CBDV).

In an embodiment, the inflorescence further comprises one or more terpenes selected from the group consisting of α-phellandrene, α-pinene, camphene, β-pinene, myrcene, limonene, eucalyptol, γ-terpinene, linalool, γ-elemene, humulene, nerolidol, guaia-3,9-diene and caryophyllene.

In another aspect disclosed herein, there is provided a method for selecting a hypoallergenic cannabis plant from a plurality of different cannabis plants, the method comprising:
a. providing a nucleic acid sample from cannabis plant tissue;
b. determining the level of expression of one or more Cannabis sativa genes, or homologs thereof, wherein the one or more genes encode a cannabis allergen;
c. comparing the level of expression determined in (b) with an allergen reference value; and
d. selecting a hypoallergenic cannabis plant based on the comparison made in (c).

The term “hypoallergenic” as used herein refers to a reduction or minimisation of the possibility of an allergic response. As used herein the terms “reduction” and “minimisation” and variation thereof such as “reduced” and “minimised” do not necessarily imply the complete reduction of the allergic response. Rather, the reduction may be to an extent, and/or for a time. Reduction may be prevention, retardation, suppression, or otherwise hindrance of the allergic response. Such reduction may be in magnitude and/or be temporal in nature. In particular contexts, the terms “reduce” and “minimise”, and variations thereof may be used interchangeably.

In an embodiment, a level of expression of the one or more genes encoding a cannabis allergen that is less than the allergen reference value is indicative that the cannabis plant is a hypoallergenic cannabis plant.

In an embodiment, the allergen reference value is representative of the level of expression of the one or more genes encoding a cannabis allergen in the cannabis plant tissue of a female cannabis plant.

In an embodiment, the cannabis allergen is selected from the group consisting of Betv1-like protein, pollen allergen, yes allergen, V5 allergen, and Par allergen.

In an embodiment, the cannabis plant tissue is inflorescence.

In an embodiment, the cannabis plant tissue is developmental Stage 4 inflorescence.

### SEQUENCE LISTING

The transcripts and sequences disclosed herein may be interchangeably defined by reference to a UniRef100 identifier, transcript identifier and sequence identifier. The sequences defined by reference to UniRef100 identifier (i.e., annotation) were current as at August 2019.

Selected transcripts have been provided in the sequence listing that accompanies the disclosure, a description of the sequences provided in the sequence listing are described in Tables 3 and 4.

TABLE 3
Description of Selected Transcript Sequences

| | | | | SEQ |
|---|---|---|---|---|
| UniRef100 Annotation | Name | Taxonomy | Transcript ID | ID NO |
| UniRef100_A0A2P5FKN5 | MADS-box transcription | Trema orientalis | Cannbio_053844 | 1 |
| | factor | | | |
| UniRef100_A0A2P5FGZ6 | Deoxyxylulose-5- | Trema orientalis | Cannbio_056731 | 2 |
| | phosphate synthase | | | |
| UniRef100_A0A2P5BJ37 | Fatty acid desaturase | Parasponia | Cannbio_056951 | 3 |
| | | andersonii | | |
| UniRef100_A0A2P5F7H7 | MADS-box transcription | Trema orientalis | Cannbio_058401 | 4 |
| | factor | | | |
| UniRef100_A0A2P5CNK4 | Pollen Ole e I family | Parasponia | Cannbio_058668 | 5 |
| | allergen protein | andersonii | | |
| UniRef100_A0A2P5DCK0 | (E,E)-geranyllinalool | Parasponia | Cannbio_059903 | 6 |
| | synthase | andersonii | | |
| UniRef100_A0A2P5FXD8 | Ves allergen | Trema orientalis | Cannbio_060030 | 7 |
| UniRef100_A0A2P5BLJ2 | Lipid transfer protein/Par | Parasponia | Cannbio_061193 | 8 |
| | allergen | andersonii | | |
| UniRef100_G9C075 | 2-C-methyl-D-erythritol | Humulus lupulus | Cannbio_062278 | 9 |
| | 2,4-cyclodiphosphate | | | |
| | synthase | | | |
| UniRef100_A0A1V0QSG6 | Terpene synthase | Cannabis sativa | Cannbio_017395 | 10 |
| UniRef100_A0A2K3NIT5 | Agamous-like mads-box | Trifolium | Cannbio_017410 | 11 |
| | protein agl8-like | pratense | | |
| | (Fragment) | | | |
| UniRef100_A0A2P5FM91 | Bet v I type allergen | Trema orientalis | Cannbio_017957 | 12 |
| UniRef100_A0A1V0QSF3 | Terpene synthase | Cannabis sativa | Cannbio_018080 | 13 |
| UniRef100_A0A1V0QSG9 | GPPS small subunit | Cannabis sativa | Cannbio_018129 | 14 |
| | (Fragment) | | | |
| UniRef100_A0A1V0QSG6 | Terpene synthase | Cannabis sativa | Cannbio_018250 | 15 |
| UniRef100_F1LKH9 | Polyketide synthase 5 | Cannabis sativa | Cannbio_018356 | 16 |
| UniRef100_A0A2P5BSA4 | Lipoxygenase | Trema orientalis | Cannbio_018948 | 17 |
| UniRef100_A0A088MFF4 | Delta 12 desaturase | Cannabis sativa | Cannbio_018959 | 18 |
| UniRef100_A0A2P5D8Y7 | Lipoxygenase | Parasponia | Cannbio_019068 | 19 |
| | | andersonii | | |
| UniRef100_A0A1V0QSH1 | Terpene synthase | Cannabis sativa | Cannbio_019267 | 20 |
| UniRef100_A7IZZ1 | (−)-limonene synthase, | Cannabis sativa | Cannbio_019445 | 21 |
| | chloroplastic | | | |
| UniRef100_A0A1V0QSF8 | Terpene synthase | Cannabis sativa | Cannbio_019641 | 22 |
| UniRef100_B1Q2B6 | 3,5,7-trioxododecanoyl- | Cannabis sativa | Cannbio_019717 | 23 |
| | CoA synthase | | | |
| UniRef100_UPI000CED6FA7 | MADS-box transcription | Morus notabilis | Cannbio_020814 | 24 |
| | factor 17 isoform X2 | | | |
| UniRef100_A0A1V0QSF8 | Terpene synthase | Cannabis sativa | Cannbio_020910 | 25 |
| UniRef100_A0A1V0QSF8 | Terpene synthase | Cannabis sativa | Cannbio_021373 | 26 |
| UniRef100_A0A1V0QSF3 | Terpene synthase | Cannabis sativa | Cannbio_021413 | 27 |
| UniRef100_A0A2P5CU97 | Major pollen allergen Lol | Parasponia | Cannbio_021476 | 28 |
| | pI | andersonii | | |
| UniRef100_W9QZH6 | Secologanin synthase | Morus notabilis | Cannbio_021743 | 29 |
| UniRef100_A0A2P5AS06 | TIR-NBS-LRR-like | Trema orientalis | Cannbio_022325 | 30 |
| | protein | | | |
| UniRef100_A0A2P5EEE4 | Fatty acid desaturase | Trema orientalis | Cannbio_022360 | 31 |
| UniRef100_W9QZH6 | Secologanin synthase | Morus notabilis | Cannbio_022533 | 32 |
| UniRef100_A7IZZ1 | (−)-limonene synthase, | Cannabis sativa | Cannbio_022649 | 33 |
| | chloroplastic | | | |
| UniRef100_A0A1V0QSH1 | Terpene synthase | Cannabis sativa | Cannbio_022866 | 34 |
| UniRef100_A0A068L6A5 | TMV resistance protein N- | Humulus lupulus | Cannbio_023213 | 35 |
| | like protein (Fragment) | | | |
| UniRef100_W9S8D7 | Vinorine synthase | Morus notabilis | Cannbio_023316 | 36 |
| UniRef100_A0A2P5AXX8 | 4-hydroxy-3-methylbut-2- | Parasponia | Cannbio_023496 | 37 |
| | en-1-yl diphosphate | andersonii | | |
| | synthase, bacterial-type | | | |
| UniRef100_A0A1V0QSG6 | Terpene synthase | Cannabis sativa | Cannbio_023581 | 38 |
| UniRef100_A0A142EGK4 | THCA synthase | Cannabis sativa | Cannbio_024022 | 39 |
| | (Fragment) | | | |
| UniRef100_A0A2P5BSA4 | Lipoxygenase | Trema orientalis | Cannbio_024416 | 40 |
| UniRef100_A0A2P5AAT5 | Cysteine-rich secretory | Trema orientalis | Cannbio_024692 | 41 |
| | protein, allergen V5/Tpx- | | | |
| | 1-related | | | |
| UniRef100_A0A2P5EEE4 | Fatty acid desaturase | Trema orientalis | Cannbio_024800 | 42 |
| UniRef100_A0A1V0QSF9 | Terpene synthase | Cannabis sativa | Cannbio_024851 | 43 |
| UniRef100_Q94LW8 | Chaicone synthase | Humulus lupulus | Cannbio_024998 | 44 |
| UniRef100_A7IZZ2 | (+)-alpha-pinene synthase, | Cannabis sativa | Cannbio_025638 | 45 |
| | chloroplastic | | | |
| UniRef100_A0A2P5BSA4 | Lipoxygenase | Trema orientalis | Cannbio_025677 | 46 |
| UniRef100_W9QZH6 | Secologanin synthase | Morus notabilis | Cannbio_025700 | 47 |
| UniRef100_A0A1V0QSF3 | Terpene synthase | Cannabis sativa | Cannbio_026189 | 48 |
| UniRef100_A5YW15 | FAD2 (Fragment) | Brassica napus | Cannbio_026331 | 49 |
| UniRef100_A0A1V0QSF8 | Terpene synthase | Cannabis sativa | Cannbio_026571 | 50 |
| UniRef100_A0A1V0QSF9 | Terpene synthase | Cannabis sativa | Cannbio_026852 | 51 |
| UniRef100_W9QMT8 | Omega-6 fatty acid | Morus notabilis | Cannbio_027028 | 52 |
| | desaturase, endoplasmic | | | |
| | reticulum isozyme 2 | | | |
| UniRef100_W9QMT8 | Omega-6 fatty acid | Morus notabilis | Cannbio_027313 | 53 |
| | desaturase, endoplasmic | | | |
| | reticulum isozyme 2 | | | |
| UniRef100_A0A2P5D8Y7 | Lipoxygenase | Parasponia | Cannbio_027834 | 54 |
| | | andersonii | | |
| UniRef100_A0A2P5CPJ2 | Pollen Ole e 1 allergen and | Trema orientalis | Cannbio_027848 | 55 |
| | extensin family protein | | | |
| UniRef100_A0A2P5BSA4 | Lipoxygenase | Trema orientalis | Cannbio_028191 | 56 |
| UniRef100_A0A2P5BSA4 | Lipoxygenase | Trema orientalis | Cannbio_028346 | 57 |
| UniRef100_A0A2P5C0X0 | MADS-box transcription | Parasponia | Cannbio_028894 | 58 |
| | factor | andersonii | | |
| UniRef100_A0A2P5EEE4 | Fatty acid desaturase | Trema orientalis | Cannbio_029003 | 59 |
| UniRef100_I6WU39 | Olivetolic acid cyclase | Cannabis sativa | Cannbio_029154 | 60 |
| UniRef100_A0A2P5ER93 | MADS-box transcription | Trema orientalis | Cannbio_029230 | 61 |
| | factor | | | |
| UniRef100_UPI0005114440 | agamous-like MADS-box | Pyrus x | Cannbio_029262 | 62 |
| | protein AGL11 | bretschneideri | | |
| UniRef100_A0A1V0QSF8 | Terpene synthase | Cannabis sativa | Cannbio_029651 | 63 |
| UniRef100_A0A2P5AIB3 | Terpene cyclase/mutase | Parasponia | Cannbio_029679 | 64 |
| | family member | andersonii | | |
| UniRef100_A0A1V0QSH1 | Terpene synthase | Cannabis sativa | Cannbio_029777 | 65 |
| UniRef100_F1LKH6 | Polyketide synthase 1 | Cannabis sativa | Cannbio_029830 | 66 |
| UniRef100_A7IZZ1 | (−)-limonene synthase, | Cannabis sativa | Cannbio_030130 | 67 |
| | chloroplastic | | | |
| UniRef100_F1LKH5 | Polyketide synthase 3 | Cannabis sativa | Cannbio_030174 | 68 |
| UniRef100_A0A2P5E973 | MADS-box transcription | Trema orientalis | Cannbio_030355 | 69 |
| | factor | | | |
| UniRef100_A0A1V0QSG3 | HDS (Fragment) | Cannabis sativa | Cannbio_030486 | 70 |
| UniRef100_A0A1V0QSG6 | Terpene synthase | Cannabis sativa | Cannbio_030713 | 71 |
| UniRef100_A0A1V0QSF8 | Terpene synthase | Cannabis sativa | Cannbio_031172 | 72 |
| UniRef100_A0A142EGK4 | THCA synthase | Cannabis sativa | Cannbio_031223 | 73 |
| | (Fragment) | | | |
| UniRef100_M9T8L0 | Fatty acid desaturase 2-1 | Linum | Cannbio_031843 | 74 |
| | | usitatissimum | | |
| UniRef100_A7IZZ2 | (+)-alpha-pinene synthase, | Cannabis sativa | Cannbio_031882 | 75 |
| | chloroplastic | | | |
| UniRef100_F1LKH9 | Polyketide synthase 5 | Cannabis sativa | Cannbio_032283 | 76 |
| UniRef100_A7IZZ1 n = 1 | (−)-limonene synthase, | Cannabis sativa | Cannbio_032659 | 77 |
| Tax = TaxID = 3483 | chloroplastic | | | |
| RepID = TPS1_CANSA | | | | |
| UniRef100_A0A1V0QSF8 | Terpene synthase | Cannabis sativa | Cannbio_032731 | 78 |
| UniRef100_A0A2P5D8Y7 | Lipoxygenase | Parasponia | Cannbio_032734 | 79 |
| | | andersonii | | |
| UniRef100_A7IZZ1 | (−)-limonene synthase, | Cannabis sativa | Cannbio_032834 | 80 |
| | chloroplastic | | | |
| UniRef100_C0LEJ7 | Fatty acid desaturase 2 | Brassica juncea | Cannbio_032875 | 81 |
| UniRef100_A0A2P5D8Y7 | Lipoxygenase | Parasponia | Cannbio_032880 | 82 |
| | | andersonii | | |
| UniRef100_A7IZZ1 | (−)-limonene synthase, | Cannabis sativa | Cannbio_032888 | 83 |
| | chloroplastic | | | |
| UniRef100_A0A1V0QSF6 | Terpene synthase | Cannabis sativa | Cannbio_033257 | 84 |
| UniRef100_A0A2P4NBR4 | Isoform 2 of mads-box | Quercus suber | Cannbio_033299 | 85 |
| | protein agl42 | | | |
| UniRef100_A7IZZ1 | (−)-limonene synthase, | Cannabis sativa | Cannbio_033300 | 86 |
| | chloroplastic | | | |
| UniRef100_A0A1V0QSF6 | Terpene synthase | Cannabis sativa | Cannbio_033443 | 87 |
| UniRef100_A0A1V0QSH1 | Terpene synthase | Cannabis sativa | Cannbio_033511 | 88 |
| UniRef100_E5RP65 | 2-acylphloroglucinol 4- | Humulus lupulus | Cannbio_033975 | 89 |
| | prenyltransferase, | | | |
| | chloroplastic | | | |
| UniRef100_F1LKH6 | Polyketide synthase 1 | Cannabis sativa | Cannbio_034051 | 90 |
| UniRef100_UPI000B78CFD1 | delta(12) -acyl-lipid- | Hevea | Cannbio_034132 | 91 |
| | desaturase-like | brasiliensis | | |
| UniRef100_A0A1V0QSH9 | HDR (Fragment) | Cannabis sativa | Cannbio_034678 | 92 |
| UniRef100_I6WU39 | Olivetolic acid cyclase | Cannabis sativa | Cannbio_034765 | 93 |
| UniRef100_A7IZZ2 | (+)-alpha-pinene synthase, | Cannabis sativa | Cannbio_034925 | 94 |
| | chloroplastic | | | |
| UniRef100_A0A1V0QSH9 | HDR (Fragment) | Cannabis sativa | Cannbio_035170 | 95 |
| UniRef100_G7J632 | Lipoxygenase | Medicago | Cannbio_035255 | 96 |
| | | truncatula | | |
| UniRef100_A0A1V0QSF8 | Terpene synthase | Cannabis sativa | Cannbio_035365 | 97 |
| UniRef100_A0A1V0QSH9 | HDR (Fragment) | Cannabis sativa | Cannbio_035649 | 98 |
| UniRef100_F1LKH7 | Polyketide synthase 2 | Cannabis sativa | Cannbio_036104 | 99 |
| UniRef100_A0A2P5BSA4 | Lipoxygenase | Trema orientalis | Cannbio_036283 | 100 |
| UniRef100_A0A1V0QSF6 | Terpene synthase | Cannabis sativa | Cannbio_036336 | 101 |
| UniRef100_A0A1V0QSH9 | HDR (Fragment) | Cannabis sativa | Cannbio_036416 | 102 |
| UniRef100_A0A1V0QSH9 | HDR (Fragment) | Cannabis sativa | Cannbio_036667 | 103 |
| UniRef100_A0A1V0QSH9 | HDR (Fragment) | Cannabis sativa | Cannbio_036684 | 104 |
| UniRef100_A0A2P5B4E8 | 4-hydroxy-3-methylbut-2- | Trema orientalis | Cannbio_036703 | 105 |
| | enyl diphosphate reductase | | | |
| UniRef100_A0A1V0QSH9 | HDR (Fragment) | Cannabis sativa | Cannbio_036741 | 106 |
| UniRef100_A0A2P5BSA4 | Lipoxygenase | Trema orientalis | Cannbio_036789 | 107 |
| UniRef100_A0A1V0QSH9 | HDR (Fragment) | Cannabis sativa | Cannbio_036932 | 108 |
| UniRef100_A7IZZ1 | (−)-limonene synthase, | Cannabis sativa | Cannbio_036960 | 109 |
| | chloroplastic | | | |
| UniRef100_A0A2P5B4E8 | 4-hydroxy-3-methylbut-2- | Trema orientalis | Cannbio_036966 | 110 |
| | enyl diphosphate reductase | | | |
| UniRef100_A0A1V0QSH9 | HDR (Fragment) | Cannabis sativa | Cannbio_037023 | 111 |
| UniRef100_A0A1V0QSH9 | HDR (Fragment) | Cannabis sativa | Cannbio_037034 | 112 |
| UniRef100_A0A1V0QSF9 | Terpene synthase | Cannabis sativa | Cannbio_037103 | 113 |
| UniRef100_F1LKH7 | Polyketide synthase 2 | Cannabis sativa | Cannbio_037193 | 114 |
| UniRef100_A0A1V0QSF6 | Terpene synthase | Cannabis sativa | Cannbio_037451 | 115 |
| UniRef100_A7IZZ1 | (−)-limonene synthase, | Cannabis sativa | Cannbio_037653 | 116 |
| | chloroplastic | | | |
| UniRef100_A0A1V0QSH9 | HDR (Fragment) | Cannabis sativa | Cannbio_037729 | 117 |
| UniRef100_A0A1V0QSF9 | Terpene synthase | Cannabis sativa | Cannbio_037841 | 118 |
| UniRef100_A0A2P5B4E8 | 4-hydroxy-3-methylbut-2- | Trema orientalis | Cannbio_037930 | 119 |
| | enyl diphosphate reductase | | | |
| UniRef100_A0A1V0QSH9 | HDR (Fragment) | Cannabis sativa | Cannbio_038048 | 120 |
| UniRef100_A0A1V0QSH9 | HDR (Fragment) | Cannabis sativa | Cannbio_038684 | 121 |
| UniRef100_A0A1V0QSH9 | HDR (Fragment) | Cannabis sativa | Cannbio_038698 | 122 |
| UniRef100_A0A1V0QSH9 | HDR (Fragment) | Cannabis sativa | Cannbio_038822 | 123 |
| UniRef100_A0A2P5E2H1 | MADS-box transcription | Parasponia | Cannbio_038827 | 124 |
| | factor | andersonii | | |
| UniRef100_A0A2P5BSA4 | Lipoxygenase | Trema orientalis | Cannbio_038873 | 125 |
| UniRef100_A0A1V0QSH9 | HDR (Fragment) | Cannabis sativa | Cannbio_038936 | 126 |
| UniRef100_A0A1V0QSG2 | Terpene synthase | Cannabis sativa | Cannbio_039060 | 127 |
| UniRef100_A0A1V0QSH9 | HDR (Fragment) | Cannabis sativa | Cannbio_039063 | 128 |
| UniRef100_A0A2P5CPJ2 | Pollen Ole e 1 allergen and | Trema orientalis | Cannbio_039084 | 129 |
| | extensin family protein | | | |
| UniRef100_A0A1V0QSH9 | HDR (Fragment) | Cannabis sativa | Cannbio_039298 | 130 |
| UniRef100_F1LKH8 | Polyketide synthase 4 | Cannabis sativa | Cannbio_039360 | 131 |
| UniRef100_F1LKH8 | Polyketide synthase 4 | Cannabis sativa | Cannbio_039530 | 132 |
| UniRef100_A0A067YB28 | Glucosyltransferase | Pueraria | Cannbio_039722 | 133 |
| | KGT15 (Fragment) | montana var. | | |
| | | lobata | | |
| UniRef100_F1LKH5 | Polyketide synthase 3 | Cannabis sativa | Cannbio_039738 | 134 |
| UniRef100_F1LKH7 | Polyketide synthase 2 | Cannabis sativa | Cannbio_039766 | 135 |
| UniRef100_A0A1V0QSF8 | Terpene synthase | Cannabis sativa | Cannbio_039793 | 136 |
| UniRef100_A7IZZ1 | (−)-limonene synthase, | Cannabis sativa | Cannbio_039860 | 137 |
| | chloroplastic | | | |
| UniRef100_A0A2P4IJF6 | Delta(12)-acyl-lipid- | Quercus suber | Cannbio_039875 | 138 |
| | desaturase | | | |
| UniRef100_F1LKH8 | Polyketide synthase 4 | Cannabis sativa | Cannbio_039981 | 139 |
| UniRef100_A0A2P4GL99 | Linoleate 13s- | Quercus suber | Cannbio_040003 | 140 |
| | lipoxygenase 2-1, | | | |
| | chloroplastic | | | |
| UniRef100_A0A2P5BSA4 | Lipoxygenase | Trema orientalis | Cannbio_040162 | 141 |
| UniRef100_A7IZZ1 | (−)-limonene synthase, | Cannabis sativa | Cannbio_040185 | 142 |
| | chloroplastic | | | |
| UniRef100_A0A1V0QSH9 | HDR (Fragment) | Cannabis sativa | Cannbio_040192 | 143 |
| UniRef100_B1Q2B6 | 3,5,7-trioxododecanoyl- | Cannabis sativa | Cannbio_041037 | 144 |
| | CoA synthase | | | |
| UniRef100_A0A1V0QSF6 | Terpene synthase | Cannabis sativa | Cannbio_041126 | 145 |
| UniRef100_A0A2P5BSA4 | Lipoxygenase | Trema orientalis | Cannbio_041476 | 146 |
| UniRef100_A0A1V0QSH9 | HDR (Fragment) | Cannabis sativa | Cannbio_041521 | 147 |
| UniRef100_A0A2P5D8Y7 | Lipoxygenase | Parasponia | Cannbio_041619 | 148 |
| | | andersonii | | |
| UniRef100_A0A2P5D8Y7 | Lipoxygenase | Parasponia | Cannbio_041633 | 149 |
| | | andersonii | | |
| UniRef100_F1LKH8 | Polyketide synthase 4 | Cannabis sativa | Cannbio_041647 | 150 |
| UniRef100_A0A1V0QSH9 | HDR (Fragment) | Cannabis sativa | Cannbio_041763 | 151 |
| UniRef100_A7IZZ1 | (−)-limonene synthase, | Cannabis sativa | Cannbio_041779 | 152 |
| | chloroplastic | | | |
| UniRef100_A0A1V0QSH9 | HDR (Fragment) | Cannabis sativa | Cannbio_041807 | 153 |
| UniRef100_A0A1V0QSH9 | HDR (Fragment) | Cannabis sativa | Cannbio_041839 | 154 |
| UniRef100_A0A2P5BSA4 | Lipoxygenase | Trema orientalis | Cannbio_042000 | 155 |
| UniRef100_A0A1V0QSF6 | Terpene synthase | Cannabis sativa | Cannbio_042088 | 156 |
| UniRef100_A0A184AGC6 | Delta 12-oleate desaturase | Brassica nigra | Cannbio_042165 | 157 |
| | (Fragment) | | | |
| UniRef100_Q6RXX0 | Delta12-oleic acid | Euphorbia | Cannbio_042391 | 158 |
| | desaturase | lagascae | | |
| UniRef100_Q6RXX0 | Delta12-oleic acid | Euphorbia | Cannbio_042452 | 159 |
| | desaturase | lagascae | | |
| UniRef100_F1LKH6 | Polyketide synthase 1 | Cannabis sativa | Cannbio_042885 | 160 |
| UniRef100_A0A2P5D2A4 | TIR-NBS-LRR-like | Parasponia | Cannbio_042929 | 161 |
| | protein | andersonii | | |
| UniRef100_A0A1V0QSG6 | Terpene synthase | Cannabis sativa | Cannbio_043040 | 162 |
| UniRef100_F1LKH9 | Polyketide synthase 5 | Cannabis sativa | Cannbio_043103 | 163 |
| UniRef100_A0A1V0QSF3 | Terpene synthase | Cannabis sativa | Cannbio_043287 | 164 |
| UniRef100_F1LKH9 | Polyketide synthase 5 | Cannabis sativa | Cannbio_043358 | 165 |
| UniRef100_A0A2P5C0X0 | MADS-box transcription | Parasponia | Cannbio_043417 | 166 |
| | factor | andersonii | | |
| UniRef100_A0A1V0QSF8 | Terpene synthase | Cannabis sativa | Cannbio_043531 | 167 |
| UniRef100_A0A2P5C0X0 | MADS-box transcription | Parasponia | Cannbio_043906 | 168 |
| | factor | andersonii | | |
| UniRef100_A0A1V0QSG2 | Terpene synthase | Cannabis sativa | Cannbio_043909 | 169 |
| UniRef100_A0A1V0QSF5 | 3-hydroxy-3- | Cannabis sativa | Cannbio_044281 | 170 |
| | methylglutaryl coenzyme | | | |
| | A reductase (Fragment | | | |
| UniRef100_I6WU39 | Olivetolic acid cyclase | Cannabis sativa | Cannbio_044427 | 171 |
| UniRef100_B1Q2B6 | 3,5,7-trioxododecanoyl- | Cannabis sativa | Cannbio_044836 | 172 |
| | CoA synthase | | | |
| UniRef100_A0A1V0QSH1 | Terpene synthase | Cannabis sativa | Cannbio_045040 | 173 |
| UniRef100_A0A088MFF4 | Delta 12 desaturase | Cannabis sativa | Cannbio_045108 | 174 |
| UniRef100_A0A142EGL9 | THCA synthase | Cannabis sativa | Cannbio_045388 | 175 |
| | (Fragment) | | | |
| UniRef100_A0A068L6A5 | TMV resistance protein N- | Humulus lupulus | Cannbio_045448 | 176 |
| | like protein (Fragment) | | | |
| UniRef100_A0A1V0QSG3 | HDS (Fragment) | Cannabis sativa | Cannbio_045663 | 177 |
| UniRef100_I6WU39 | Olivetolic acid cyclase | Cannabis sativa | Cannbio_045994 | 178 |
| UniRef100_A0A1V0QSF3 | Terpene synthase | Cannabis sativa | Cannbio_046296 | 179 |
| UniRef100_A0A2P5B4E8 | 4-hydroxy-3-methylbut-2- | Trema orientalis | Cannbio_046662 | 180 |
| | enyl diphosphate reductase | | | |
| UniRef100_A0A2P5FJB2 | Major pollen allergen Lol | Trema orientalis | Cannbio_046671 | 181 |
| | pI | | | |
| UniRef100_A0A2P5BSA4 | Lipoxygenase | Trema orientalis | Cannbio_046768 | 182 |
| UniRef100_A0A2P5BSA4 | Lipoxygenase | Trema orientalis | Cannbio_046769 | 183 |
| UniRef100_A0A1Q3B5J9 | FA_desaturase domain- | Cephalotus | Cannbio_047069 | 184 |
| | containing | follicularis | | |
| | protein/DUF3474 domain- | | | |
| | containing protein | | | |
| UniRef100_W9QMT8 | Omega-6 fatty acid | Morus notabilis | Cannbio_047523 | 185 |
| | desaturase, endoplasmic | | | |
| | reticulum isozyme 2 | | | |
| UniRef100_UPI000B79458D | omega-6 fatty acid | Chenopodium | Cannbio_047604 | 186 |
| | desaturase, endoplasmic | quinoa | | |
| | reticulum isozyme 1-like | | | |
| UniRef100_A0A2P5D7B0 | MADS-box transcription | Trema orientalis | Cannbio_047696 | 187 |
| | factor | | | |
| UniRef100_Q6RXX0 | Deltal2-oleic acid | Euphorbia | Cannbio_047742 | 188 |
| | desaturase | lagascae | | |
| UniRef100_A0A2P5CKQ2 | Allergen Ole e 1, | Parasponia | Cannbio_048042 | 189 |
| | conserved site | andersonii | | |
| UniRef100_A0A068L6A5 | TMV resistance protein N- | Humulus lupulus | Cannbio_048101 | 190 |
| | like protein (Fragment) | | | |
| UniRef100_A0A1V0QSG2 | Terpene synthase | Cannabis sativa | Cannbio_048110 | 191 |
| UniRef100_A0A1V0QSG2 | Terpene synthase | Cannabis sativa | Cannbio_048188 | 192 |
| UniRef100_A0A1V0QSF8 | Terpene synthase | Cannabis sativa | Cannbio_048198 | 193 |
| UniRef100_F1LKH6 | Polyketide synthase 1 | Cannabis sativa | Cannbio_048260 | 194 |
| UniRef100_B1Q2B6 | 3,5,7-trioxododecanoyl- | Cannabis sativa | Cannbio_048356 | 195 |
| | CoA synthase | | | |
| UniRef100_F1LKH8 | F1LKH8 Polyketide | Cannabis sativa | Cannbio_000101 | 196 |
| | synthase 4 | | | |
| UniRef100_I6WU39 | Olivetolic acid cyclase | Cannabis sativa | Cannbio_000163 | 197 |
| UniRef100_A0A2P5BB86 | TIR-NBS-LRR-like | Trema orientalis | Cannbio_000445 | 198 |
| | protein | | | |
| UniRef100_A0A1V0QSF9 | Terpene synthase | Cannabis sativa | Cannbio_000746 | 199 |
| UniRef100_A0A1V0QSH9 | HDR (Fragment) | Cannabis sativa | Cannbio_000796 | 200 |
| UniRef100_A0A142EGM0 | Truncated THCA synthase | Cannabis sativa | Cannbio_000877 | 201 |
| UniRef100_A0A1V0QSF8 | Terpene synthase | Cannabis sativa | Cannbio_001051 | 202 |
| UniRef100_A0A1V0QSH9 | HDR (Fragment) | Cannabis sativa | Cannbio_001191 | 203 |
| UniRef100_A0A2P5B4E8 | 4-hydroxy-3-methylbut-2- | Trema orientalis | Cannbio_001253 | 204 |
| | enyl diphosphate reductase | | | |
| UniRef100_A0A2P5E2H1 | MADS-box transcription | Parasponia | Cannbio_001292 | 205 |
| | factor | andersonii | | |
| UniRef100_A0A2P5B4E8 | 4-hydroxy-3-methylbut-2- | Trema orientalis | Cannbio_001301 | 206 |
| | enyl diphosphate reductase | | | |
| UniRef100_A0A2P5CKQ2 | Allergen Ole e 1, | Parasponia | Cannbio_001307 | 207 |
| | conserved site | andersonii | | |
| UniRef100_A0A1V0QSG2 | Terpene synthase | Cannabis sativa | Cannbio_001375 | 208 |
| UniRef100_B1Q2B6 | 3,5,7-trioxododecanoyl- | Cannabis sativa | Cannbio_001409 | 209 |
| | CoA synthase | | | |
| UniRef100_W9R118 | 4-hydroxy-3-methylbut-2- | Morus notabilis | Cannbio_001432 | 210 |
| | enyl diphosphate reductase | | | |
| UniRef100_A0A1V0QSH9 | HDR (Fragment) | Cannabis sativa | Cannbio_001482 | 211 |
| UniRef100_A0A1V0QSH9 | HDR (Fragment) | Cannabis sativa | Cannbio_001569 | 212 |
| UniRef100_A0A218ANK1 | Omega-6 fatty acid | Idesia polycarpa | Cannbio_001610 | 213 |
| | desaturase | | | |
| UniRef100_F1LKH8 | Polyketide synthase 4 | Cannabis sativa | Cannbio_001628 | 214 |
| UniRef100_A0A1V0QSH9 | HDR (Fragment) | Cannabis sativa | Cannbio_001663 | 215 |
| UniRef100_A0A1V0QSH9 | HDR (Fragment) | Cannabis sativa | Cannbio_001692 | 216 |
| UniRef100_UPI00090D6990 | omega-6 fatty acid | Lupinus | Cannbio_001709 | 217 |
| | desaturase, endoplasmic | angustifolius | | |
| | reticulum isozyme 1-like | | | |
| UniRef100_A0A1V0QSH9 | HDR (Fragment) | Cannabis sativa | Cannbio_001749 | 218 |
| UniRef100_F1LKH7 | Polyketide synthase 2 | Cannabis sativa | Cannbio_001757 | 219 |
| UniRef100_A0A1V0QSH9 | HDR (Fragment) | Cannabis sativa | Cannbio_001860 | 220 |
| UniRef100_A0A2P5BSA4 | Lipoxygenase | Trema orientalis | Cannbio_002094 | 221 |
| UniRef100_A0A1V0QSH9 | HDR (Fragment) | Cannabis sativa | Cannbio_002181 | 222 |
| UniRef100_A0A1V0QSH9 | HDR (Fragment) | Cannabis sativa | Cannbio_002273 | 223 |
| UniRef100_A0A1Q3B5J9 | FA_desaturase domain- | Cephalotus | Cannbio_002293 | 224 |
| n = 1 | containing | follicularis | | |
| | protein/DUF3474 domain- | | | |
| | containing protein | | | |
| UniRef100_A0A142EGL4 | Truncated THCA synthase | Cannabis sativa | Cannbio_002736 | 225 |
| UniRef100_A0A2P5D8Y7 | Lipoxygenase | Parasponia | Cannbio_002844 | 226 |
| | | andersonii | | |
| UniRef100_A0A2P5E2H1 | MADS-box transcription | Parasponia | Cannbio_002936 | 227 |
| | factor | andersonii | | |
| UniRef100_W9QZH6 | Secologanin synthase | Morus notabilis | Cannbio_003185 | 228 |
| UniRef100_W9QMT8 | Omega-6 fatty acid | Morus notabilis | Cannbio_003201 | 229 |
| | desaturase, endoplasmic | | | |
| | reticulum isozyme 2 | | | |
| UniRef100_A0A1V0QSF3 | Terpene synthase | Cannabis sativa | Cannbio_004639 | 230 |
| UniRef100_A0A1V0QSG2 | Terpene synthase | Cannabis sativa | Cannbio_004731 | 231 |
| UniRef100_A0A1V0QSH9 | HDR (Fragment) | Cannabis sativa | Cannbio_004871 | 232 |
| UniRef100_B1Q2B6 | 3,5,7-trioxododecanoyl- | Cannabis sativa | Cannbio_004873 | 233 |
| | CoA synthase | | | |
| UniRef100_A0A142EGM0 | Truncated THCA synthase | Cannabis sativa | Cannbio_005228 | 234 |
| UniRef100_A0A1W5Q131 | Delta-12 fatty acid | Perilla | Cannbio_005992 | 235 |
| | desaturase allele 2 | frutescens | | |
| UniRef100_A0A1V0QSF6 | Terpene synthase | Cannabis sativa | Cannbio_006127 | 236 |
| UniRef100_A0A1V0QSF6 | Terpene synthase | Cannabis sativa | Cannbio_006183 | 237 |
| UniRef100_A0A2P5BJ37 | Fatty acid desaturase | Parasponia | Cannbio_006213 | 238 |
| | | andersonii | | |
| UniRef100_A0A2P5CS91 | MADS-box transcription | Trema orientalis | Cannbio_006389 | 239 |
| | factor | | | |
| UniRef100_A0A1V0QSG6 | Terpene synthase | Cannabis sativa | Cannbio_006417 | 240 |
| UniRef100_A0A1V0QSF3 | Terpene synthase | Cannabis sativa | Cannbio_006443 | 241 |
| UniRef100_A0A1V0QSG6 | Terpene synthase | Cannabis sativa | Cannbio_006553 | 242 |
| UniRef100_A0A1V0QSG2 | Terpene synthase | Cannabis sativa | Cannbio_006565 | 243 |
| UniRef100_A0A1V0QSH9 | HDR (Fragment) | Cannabis sativa | Cannbio_006736 | 244 |
| UniRef100_A7IZZ1 | (−)-limonene synthase, | Cannabis sativa | Cannbio_006811 | 245 |
| | chloroplastic | | | |
| UniRef100_A0A1V0QSF8 | Terpene synthase | Cannabis sativa | Cannbio_006875 | 246 |
| UniRef100_B1Q2B6 | 3,5,7-trioxododecanoyl- | Cannabis sativa | Cannbio_008449 | 247 |
| | CoA synthase | | | |
| UniRef100_A0A2P5AS06 | TIR-NBS-LRR-like | Trema orientalis | Cannbio_008463 | 248 |
| | protein | | | |
| UniRef100_A0A1V0QSG2 | Terpene synthase | Cannabis sativa | Cannbio_009119 | 249 |
| UniRef100_A0A2P5FUU5 | MADS-box transcription | Trema orientalis | Cannbio_009169 | 250 |
| | factor | | | |
| UniRef100_F1LKH8 | Polyketide synthase 4 | Cannabis sativa | Cannbio_009189 | 251 |
| UniRef100_A0A142EGL4 | Truncated THCA synthase | Cannabis sativa | Cannbio_009678 | 252 |
| UniRef100_A0A2P5F1B3 | MADS-box transcription | Trema orientalis | Cannbio_009872 | 253 |
| | factor | | | |
| UniRef100_A0A2P5FZN0 | Bet v I type allergen | Trema orientalis | Cannbio_010274 | 254 |
| UniRef100_A0A2P5DKL8 | Pollen allergen Ole e 1 | Trema orientalis | Cannbio_010479 | 255 |
| | family | | | |
| UniRef100_A0A2P5E2H1 | MADS-box transcription | Parasponia | Cannbio_010630 | 256 |
| | factor | andersonii | | |
| UniRef100_A0A2P5BJ37 | Fatty acid desaturase | Parasponia | Cannbio_010956 | 257 |
| | | andersonii | | |
| UniRef100_A7IZZ1 | (−)-limonene synthase, | Cannabis sativa | Cannbio_012008 | 258 |
| | chloroplastic | | | |
| UniRef100_A0A1V0QSF3 | Terpene synthase | Cannabis sativa | Cannbio_012506 | 259 |
| UniRef100_A0A1Q3CCU4 | Terpene cyclase/mutase | Cephalotus | Cannbio_012536 | 260 |
| | family member | follicularis | | |
| UniRef100_W9RC97 | Vinorine synthase | Mortis notabilis | Cannbio_012638 | 261 |
| UniRef100_A0A1V0QSF9 | Terpene synthase | Cannabis sativa | Cannbio_012855 | 262 |
| UniRef100_A0A2P5FJP0 | Ves allergen | Trema orientalis | Cannbio_013018 | 263 |
| UniRef100_A0A2P5A3W2 | MADS-box transcription | Parasponia | Cannbio_013204 | 264 |
| | factor | andersonii | | |
| UniRef100_A0A2P5CGN5 | MADS-box transcription | Trema orientalis | Cannbio_013615 | 265 |
| | factor | | | |
| UniRef100_A6P6W0 | Cannabidiolic acid | Cannabis sativa | Cannbio_013699 | 266 |
| | synthase-like 1 | | | |
| UniRef100_A0A2P5FJB2 | Major pollen allergen Lol | Trema orientalis | Cannbio_013730 | 267 |
| | pI | | | |
| UniRef100_A0A1V0QSG2 | Terpene synthase | Cannabis sativa | Cannbio_013743 | 268 |
| UniRef100_A0A2P5FJB2 | Major pollen allergen Lol | Trema orientalis | Cannbio_013884 | 269 |
| | pI | | | |
| UniRef100_A0A2P5BJY5 | MADS-box transcription | Parasponia | Cannbio_013942 | 270 |
| | factor | andersonii | | |
| UniRef100_A0A1V0QSF8 | Terpene synthase | Cannabis sativa | Cannbio_014047 | 271 |
| UniRef100_W9QZH6 | Secologanin synthase | Mortis notabilis | Cannbio_014174 | 272 |
| UniRef100_A0A2P5CJH3 | Major pollen allergen Lol | Parasponia | Cannbio_014187 | 273 |
| | pI | andersonii | | |
| UniRef100_A0A2P5F216 | Pollen Ole e 1 allergen and | Trema orientalis | Cannbio_014514 | 274 |
| | extensin family protein | | | |
| UniRef100_A0A2P5FNX9 | MADS-box transcription | Trema orientalis | Cannbio_014948 | 275 |
| | factor | | | |
| UniRef100_I1V0C6 | Tetrahydrocannabinolic | Cannabis sativa | Cannbio_014959 | 276 |
| | acid synthase (Fragment) | | | |
| UniRef100_A0A2P5BSA4 | Lipoxygenase | Trema orientalis | Cannbio_015133 | 277 |
| UniRef100_B6SCF6 | Germacrene-A synthase | Humulus lupulus | Cannbio_015144 | 278 |
| UniRef100_A0A2P5AD11 | NB-ARC domain, LRR | Parasponia | Cannbio_015516 | 279 |
| | domain containing protein | andersonii | | |
| UniRef100_A0A1V0QSG3 | HDS (Fragment) | Cannabis sativa | Cannbio_015609 | 280 |
| UniRef100_Q94LW8 | Chaicone synthase | Humulus lupulus | Cannbio_015624 | 281 |
| UniRef100_A0A2P5BB86 | TIR-NBS-LRR-like | Trema orientalis | Cannbio_015748 | 282 |
| | protein | | | |
| UniRef100_A0A088MFF4 | Delta 12 desaturase | Cannabis sativa | Cannbio_015972 | 283 |
| UniRef100_A7IZZ2 | (+)-alpha-pinene synthase, | Cannabis sativa | Cannbio_016048 | 284 |
| | chloroplastic | | | |
| UniRef100_A6P6W0 | Cannabidiolic acid | Cannabis sativa | Cannbio_016114 | 285 |
| | synthase-like 1 | | | |
| UniRef100_A6P6V9 | Cannabidiolic acid | Cannabis sativa | Cannbio_016204 | 286 |
| | synthase | | | |
| UniRef100_A0A1V0QSF3 | Terpene synthase | Cannabis sativa | Cannbio_016317 | 287 |
| UniRef100_A0A1V0QSG2 | Terpene synthase | Cannabis sativa | Cannbio_016394 | 288 |
| UniRef100_A0A2P5E749 | Terpene cyclase/mutase | Trema orientalis | Cannbio_016508 | 289 |
| | family member | | | |
| UniRef100_A6P6V9 | Cannabidiolic acid | Cannabis sativa | Cannbio_016865 | 290 |
| | synthase | | | |
| UniRef100_A0A2P5FDA0 | MADS-box transcription | Trema orientalis | Cannbio_053021 | 291 |
| | factor | | | |
| UniRef100_A0A2P5FDA0 | MADS-box transcription | Trema orientalis | Cannbio_048850 | 292 |
| | factor | | | |
| UniRef100_A0A088MFF4 | Delta 12 desaturase | Cannabis sativa | Cannbio_048952 | 293 |
| UniRef100_A0A2P5CSN3 | MADS-box transcription | Trema orientalis | Cannbio_049455 | 294 |
| | factor | | | |
| UniRef100_A0A2P5EYN7 | MADS-box transcription | Trema orientalis | Cannbio_050616 | 295 |
| | factor | | | |
| UniRef100_A0A2P5D9J6 | MADS-box transcription | Parasponia | Cannbio_050725 | 296 |
| | factor | andersonii | | |
| UniRef100_A0A2P5E2H1 | MADS-box transcription | Parasponia | Cannbio_052623 | 297 |
| | factor | andersonii | | |
| UniRef100_UPI000CECED | 97 lupeol synthase | Mortis notabilis | Cannbio_049822 | 298 |
| UniRef100_UPI000CED62 | 77 lupeol synthase isoform | Mortis notabilis | Cannbio_049974 | 299 |
| | X2 | | | |
| UniRef100_A0A2P5F235 | TIR-NBS-LRR-like | Trema orientalis | Cannbio_052701 | 300 |
| | protein | | | |
| UniRef100_A0A2P5FAX7 | Pollen allergen ole e | Trema orientalis | Cannbio_048504 | 301 |
| UniRef100_A0A2P5F7H7 | MADS-box transcription | Trema orientalis | Cannbio_057623 | 302 |
| | factor | | | |
| UniRef100_A0A2P5FXD8 | Ves allergen | Trema orientalis | Cannbio_058961 | 303 |
| UniRef100_A0A1V0QSH9 | HDR (Fragment) | Cannabis sativa | Cannbio_060627 | 304 |
| UniRef100_A0A2P5B4E8 | 4-hydroxy-3-methylbut-2- | Trema orientalis | Cannbio_061752 | 305 |
| | enyl diphosphate reductase | | | |
| UniRef100_UPI000CE16E | CB agamous-like MADS- | Quercus suber | Cannbio_062122 | 306 |
| | box protein AGE 104 | | | |
| UniRef100_A0A2P5FH55 | MADS-box transcription | Trema orientalis | Cannbio_062967 | 307 |
| | factor | | | |
| UniRef100_A0A2P5FUU5 | MADS-box transcription | Trema orientalis | Cannbio_062969 | 308 |
| | factor | | | |
| UniRef100_A0A2P5AD11 | NB-ARC domain, ERR | Parasponia | Cannbio_063072 | 309 |
| | domain containing protein | andersonii | | |
| UniRef100_A0A2P5EEE4 | Fatty acid desaturase | Trema orientalis | Cannbio_063081 | 310 |
| UniRef100_A0A2P5EQJ8 | Major pollen allergen Lol | Trema orientalis | Cannbio_064056 | 311 |
| | pI | | | |
| UniRef100_A0A2P5AD11 | NB-ARC domain, LRR | Parasponia | Cannbio_064155 | 312 |
| | domain containing protein | andersonii | | |

TABLE 4
Description of Selected Variant Sequences.

| Transcript ID | Variant SEQ ID NO |
|---|---|
| Cannbio_053844 | 313 |
| Cannbio_056731 | 314 |
| Cannbio_056951 | 315 |
| Cannbio_058401 | 316 |
| Cannbio_058668 | 317 |
| Cannbio_059903 | 318 |
| Cannbio_060030 | 319 |
| Cannbio_061193 | 320 |
| Cannbio_062278 | 321 |
| Cannbio_017395 | 322 |
| Cannbio_017410 | 323 |
| Cannbio_017957 | 324 |
| Cannbio_018129 | 325 |
| Cannbio_018250 | 326 |
| Cannbio_018356 | 327 |
| Cannbio_018948 | 328 |
| Cannbio_018959 | 329 |
| Cannbio_019445 | 330 |
| Cannbio_019717 | 331 |
| Cannbio_020910 | 332 |
| Cannbio_021476 | 333 |
| Cannbio_021743 | 334 |
| Cannbio_022325 | 335 |
| Cannbio_022360 | 336 |
| Cannbio_022533 | 337 |
| Cannbio_022866 | 338 |
| Cannbio_023213 | 339 |
| Cannbio_023316 | 340 |
| Cannbio_023581 | 341 |
| Cannbio_024692 | 342 |
| Cannbio_024851 | 343 |
| Cannbio_024998 | 344 |
| Cannbio_025677 | 345 |
| Cannbio_025700 | 346 |
| Cannbio_026189 | 347 |
| Cannbio_026331 | 348 |
| Cannbio_026571 | 349 |
| Cannbio_026852 | 350 |
| Cannbio_027028 | 351 |
| Cannbio_027313 | 352 |
| Cannbio_027834 | 353 |
| Cannbio_027848 | 354 |
| Cannbio_028346 | 355 |
| Cannbio_029003 | 356 |
| Cannbio_029154 | 357 |
| Cannbio_029777 | 358 |
| Cannbio_030130 | 359 |
| Cannbio_030174 | 360 |
| Cannbio_030355 | 361 |
| Cannbio_030486 | 362 |
| Cannbio_030713 | 363 |
| Cannbio_031172 | 364 |
| Cannbio_031843 | 365 |
| Cannbio_032283 | 366 |
| Cannbio_032659 | 367 |
| Cannbio_032731 | 368 |
| Cannbio_032834 | 369 |
| Cannbio_032875 | 370 |
| Cannbio_032888 | 371 |
| Cannbio_033299 | 372 |
| Cannbio_033443 | 373 |
| Cannbio_033511 | 374 |
| Cannbio_034051 | 375 |
| Cannbio_034132 | 376 |
| Cannbio_034765 | 377 |
| Cannbio_034925 | 378 |
| Cannbio_035365 | 379 |
| Cannbio_036336 | 380 |
| Cannbio_036703 | 381 |
| Cannbio_036789 | 382 |
| Cannbio_036960 | 383 |
| Cannbio_037930 | 384 |
| Cannbio_038936 | 385 |
| Cannbio_039084 | 386 |
| Cannbio_039530 | 387 |
| Cannbio_039860 | 388 |
| Cannbio_039875 | 389 |
| Cannbio_039981 | 390 |
| Cannbio_040185 | 391 |
| Cannbio_041037 | 392 |
| Cannbio_041126 | 393 |
| Cannbio_041476 | 394 |
| Cannbio_042165 | 395 |
| Cannbio_042391 | 396 |
| Cannbio_042452 | 397 |
| Cannbio_042885 | 398 |
| Cannbio_042929 | 399 |
| Cannbio_043040 | 400 |
| Cannbio_043103 | 401 |
| Cannbio_043287 | 402 |
| Cannbio_043358 | 403 |
| Cannbio_043531 | 404 |
| Cannbio_043909 | 405 |
| Cannbio_044281 | 406 |
| Cannbio_044427 | 407 |
| Cannbio_045040 | 408 |
| Cannbio_045108 | 409 |
| Cannbio_045388 | 410 |
| Cannbio_045994 | 411 |
| Cannbio_046296 | 412 |
| Cannbio_046671 | 413 |
| Cannbio_046768 | 414 |
| Cannbio_046769 | 415 |
| Cannbio_047069 | 416 |
| Cannbio_047523 | 417 |
| Cannbio_047604 | 418 |
| Cannbio_047742 | 419 |
| Cannbio_048042 | 420 |
| Cannbio_048110 | 421 |
| Cannbio_048188 | 422 |
| Cannbio_048198 | 423 |
| Cannbio_048260 | 424 |
| Cannbio_048356 | 425 |
| Cannbio_000101 | 426 |
| Cannbio_000163 | 427 |
| Cannbio_000445 | 428 |
| Cannbio_000746 | 429 |
| Cannbio_000877 | 430 |
| Cannbio_001051 | 431 |
| Cannbio_001253 | 432 |
| Cannbio_001292 | 433 |
| Cannbio_001307 | 434 |
| Cannbio_001610 | 435 |
| Cannbio_001628 | 436 |
| Cannbio_001709 | 437 |
| Cannbio_002293 | 438 |
| Cannbio_002736 | 439 |
| Cannbio_002936 | 440 |
| Cannbio_003185 | 441 |
| Cannbio_004639 | 442 |
| Cannbio_004731 | 443 |
| Cannbio_004873 | 444 |
| Cannbio_005228 | 445 |
| Cannbio_005992 | 446 |
| Cannbio_006127 | 447 |
| Cannbio_006183 | 448 |
| Cannbio_006213 | 449 |
| Cannbio_006389 | 450 |
| Cannbio_006417 | 451 |
| Cannbio_006443 | 452 |
| Cannbio_006553 | 453 |
| Cannbio_006565 | 454 |
| Cannbio_006811 | 455 |
| Cannbio_006875 | 456 |
| Cannbio_008449 | 457 |
| Cannbio_008463 | 458 |
| Cannbio_009119 | 459 |
| Cannbio_009169 | 460 |
| Cannbio_009189 | 461 |
| Cannbio_009678 | 462 |
| Cannbio_009872 | 463 |
| Cannbio_010274 | 464 |
| Cannbio_010479 | 465 |
| Cannbio_010630 | 466 |
| Cannbio_010956 | 467 |
| Cannbio_012008 | 468 |
| Cannbio_012506 | 469 |
| Cannbio_012536 | 470 |
| Cannbio_012638 | 471 |
| Cannbio_012855 | 472 |
| Cannbio_013018 | 473 |
| Cannbio_013204 | 474 |
| Cannbio_013615 | 475 |
| Cannbio_013699 | 476 |
| Cannbio_013730 | 477 |
| Cannbio_013743 | 478 |
| Cannbio_013884 | 479 |
| Cannbio_013942 | 480 |
| Cannbio_014047 | 481 |
| Cannbio_014174 | 482 |
| Cannbio_014187 | 483 |
| Cannbio_014514 | 484 |
| Cannbio_014948 | 485 |
| Cannbio_014959 | 486 |
| Cannbio_015133 | 487 |
| Cannbio_015144 | 488 |
| Cannbio_015516 | 489 |
| Cannbio_015609 | 490 |
| Cannbio_015624 | 491 |
| Cannbio_015748 | 492 |
| Cannbio_015972 | 493 |
| Cannbio_016048 | 494 |
| Cannbio_016114 | 495 |
| Cannbio_016204 | 496 |
| Cannbio_016317 | 497 |
| Cannbio_016394 | 498 |
| Cannbio_016508 | 499 |
| Cannbio_016865 | 500 |
| Cannbio_053021 | 501 |
| Cannbio_048850 | 502 |
| Cannbio_048952 | 503 |
| Cannbio_049455 | 504 |
| Cannbio_050616 | 505 |
| Cannbio_050725 | 506 |
| Cannbio_052623 | 507 |
| Cannbio_049822 | 508 |
| Cannbio_049974 | 509 |
| Cannbio_052701 | 510 |
| Cannbio_048504 | 511 |
| Cannbio_057623 | 512 |
| Cannbio_058961 | 513 |
| Cannbio_061752 | 514 |
| Cannbio_062122 | 515 |
| Cannbio_062967 | 516 |
| Cannbio_062969 | 517 |
| Cannbio_063072 | 518 |
| Cannbio_063081 | 519 |
| Cannbio_064056 | 520 |
| Cannbio_064155 | 521 |

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

The various embodiments enabled herein are further described by the following non-limiting examples.

### EXAMPLES

### A. Materials

### Plant Material

Cannabis plants were grown under an Office of Drug Control license at the Victorian Government Medicinal Cannabis Cultivation Facility, Victoria, Australia. Indoor greenhouse growing facilities were equipped with full climate control (i.e., temperature, humidity and high-intensity lighting) to ensure that crops were produced in almost identical growing conditions.

Cannabis plants were asexually propagated from cuttings taken from vegetative mother plants originating from a single seed source. Cuttings were maintained for 2 weeks at 22° C. in a high humidity environment (i.e., 50% relative humidity) under 18 hours day light in rooting medium to stimulate root development before being transferred to substrate medium for hydroponic growth. The plants were grown for a further 5 weeks under the same growth conditions before being transferred to a larger substrate medium to induce flowering.

Flowering conditions were identical to the rooting and growth conditions, with the exception that the daylight length was reduced to 12 hours. The plants were maintained in flowering conditions for 9 weeks to allow for flowering and maturation. The plants were irrigated throughout their growing cycle with potable quality water and sustained release fertilizer was applied to the soil-free medium.

A female cannabis strain and male cannabis strain were maintained under these conditions.

The female cannabis strain used for the purpose of these analyses has a cannabinoid profile enriched for total CBD and total THC, as provided by Table 5, below (mg/g).

TABLE 5
Quantitative analysis of cannabinoids in
CBD- and THC-enriched female cannabis.

| | | | | | | | | Total |
|---|---|---|---|---|---|---|---|---|
| | | | | | | | | canna- |
| Strain | CBD | THC | CBG | CBC | CBN | CBDV | THCV | binoid |
| Female | 53.33 | 33.96 | 1.21 | 3.12 | 0.1 | 0.23 | 0.24 | 92.19 |

The terpene profile of the female cannabis strain is also characterised by enrichment for myrcene and β-pinene. The relative abundance (ratio) of myrcene to β-pinene in the female cannabis strain is from about 40:1 to about 1:1.

### B. Sample Preparation

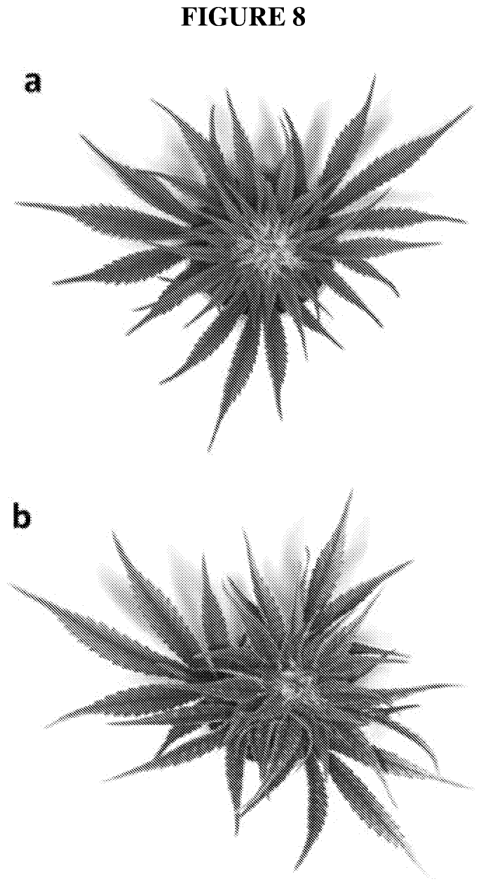

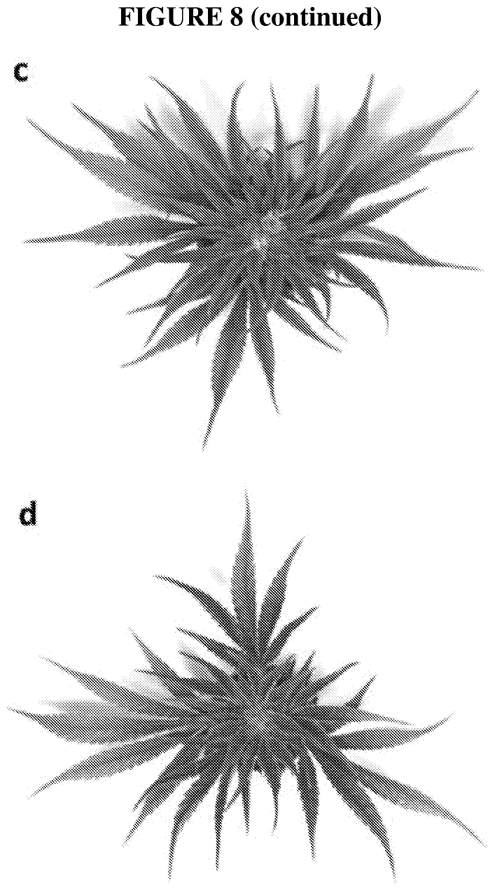

Plant tissues from multiple sources were sampled including stem, root-tip, root-mid, leaf tissue at various developmental stages of the plant that ranged from a freshly planted cutting, vegetative plant to reproductive plant. To study the expression level of the cannabinoid biosynthesis pathway genes, floral bud tissues and trichomes were isolated from reproductive plants at four different timepoints, in six biological replicates. The four timepoints included tissues harvested at 35, 42, 49 and 56 days after induction of flowering in the female plants (FIG. 8). In addition, vegetative leaf and reproductive tissues (pollen sacs) were harvested from the male strain plant.

Trichomes were harvested from the female floral buds using the method described previously (Vincent et al. Molecules. 2019, 24(4): E659) with some modifications. Harvested floral bud tissue (— 3-5 cm×3-5 cm) was placed in a Falcon 50 mL tube filled with 20% of liquid nitrogen. The tube was loosely capped and vortexed for a maximum of 2 min to dislodge the trichomes onto the sides of the tube. The remaining tissue was removed manually from the tube by forceps and the released trichomes were gently resuspended in 1 mL of the lysis buffer from the RNeasy® Plant Mini Kit (QIAGEN, Hilden, Germany). The resuspended tissue was filtered through the cell strainer (180 microns) to further purify the trichomes which were immediately processed for extraction of RNA.

### C. Total RNA Extraction and RNA-Seq Library Preparation

For RNA extraction of trichomes and all other harvested samples of the plant, total RNA was extracted using the RNeasy® Plant Mini Kit (QIAGEN, Hilden, Germany) following manufacturer's instructions. The concentration of RNA was confirmed using a spectrophotometer (Thermo Scientific, Wilmington, Del., USA) at the wavelength ratios of A260/230 and A260/280 nm.

RNA-Seq libraries were prepared with the SureSelect Strand-Specific RNA Library Kit (Agilent Technologies, Santa Clara, Calif., USA) according to manufacturer's instructions. Each library was prepared with a unique indexing primer. The libraries were assessed for quality and quantification purposes on an Agilent TapeStation 2200 platform with D1000 ScreenTape (Agilent Technologies, Santa Clara, Calif., USA) following the manufacturer's protocol. RNA-Seq libraries were multiplexed in an equimolar concentration to generate a single pool. The multiplexed pooled sample was quantified using the high-sensitivity fluorometric assay (Qubit, Thermo Fisher Scientific, Waltham, U.S.A.) according to the protocol described by the manufacturer. The quantified sample was subjected to 2×150 pair-end sequencing using the HiSeq 3000 system (Illumina Inc., San Diego, Calif., USA).

### D. Sequence Data Processing and De Novo Assembly

The raw reads of sequences were filtered by employing a custom perl script and Cutadapt v. 1.9 (Martin, EMBnet.journal. 2011, 17: 10-12). Adaptor sequences and low-quality reads (reads with >10% bases with Q≤20) were removed from the resulting data. Trimming of the data involved removal of the reads that had three or more consecutive unassigned Ns with a phred score of ≤20. Sequence reads that were less than 50 bp were discarded prior to the de novo transcriptome assembly step. The filtered data was assembled using the transcriptome assembler, SOAPdenovo-TRANS (REF 45) with k-mer size of 51, 69, 73, 75, 91 and 101 to find the optimum k-mer size for the assembly. The resulting contigs and scaffolds from the chosen k-mer size assembly that had a total length of less than 240 bp were omitted, as these were considered shorter than the length of a single pair of the sequence. Transcripts that ranged between 240-500 bp in length and had less than 10 sequence reads associated with the assembly were also discarded. To generate more complete sequences with longer length, fork, bubble and complex loci from SOAPdenovo-TRANS assembly were further combined using the CAPS assembler (Huang & Madan, Genome Res. 1999, 9: 869-877) with 95% identity and minimum overlap of 50 bp.

### E. Transcriptome Annotation

The generated transcriptome assembly was compared using BLASTX (Altschul et al. Nucleic Acids Res. 1997, 25: 3389-3402) against the UniRef100 database (Suzek et al. Bioinformatics. 2007, 23: 1282-1288) with the threshold E-value of <10⁻¹⁰. The transcripts were further BLASTN analysed against the previously-generated cannabis transcriptome databases of PK and Finola (van Bakal et al. supra) and to the CDS of CBDrx genome assembly (Grassa et al. supra). Transcripts that displayed a significant match to non-plant databases based on their annotation were removed from further analysis. The assembled transcripts were also assigned gene ontology (GO) terms based on sequence similarity to UniRef100 database. GO terms were retrieved based on UniRef100 identifiers (i.e., annotations) using Retrieve/ID mapping tool of UniProt and their distribution across categories was compared and plotted using WEGO (Ye et al. Nucleic Acids Res. 2006, 34: 293-297; Zhou et al. Nucleic Acids Res. 2018, 46: 71-75).

### F. Differential Gene Expression Analysis

To analyse differential gene expression, quality trimmed sequence reads from each of the tissue sample were aligned to the generated transcriptome assembly using the BWA-MEM software package (Li. arXiv Preprint. 2013, 1303.3997) using default parameters. Overall transcriptional activity was determined by normalising read counts using the DESeq method (Anders & Huber. Genome Biol. 2010, 11: 106). Principal component analysis (PCA) plot was utilised to visualise and assess the clustering of the data. R Bioconductor package, DESeq2 (Love et al. Genome Biol. 2014, 15: 550) was used to perform differential gene expression analysis. Benjamini-Hochberg method was used to control the false discovery rate (FDR) by adjusting the p-values (Benjamini & Hochberg. J. Royal Statist. Soc., Series B. 1995, 57: 289-300). Genes were included for further analysis only if they were defined to be significantly differentially expressed; if the value for Log₂ fold changes were either ≥two-fold or ≤-two-fold with adjusted p-value (Padj) of ≤0.05.

The differential expression analysis was carried out separately for the two variables of tissue type and female floral stage-specific development. To study the differential gene expression across multiple tissue types, the samples were categorised into leaf/stem and root tissues from vegetative plant and reproductive tissues of male and female plants (floral buds with trichomes and trichome tissue). For the study of differential expression of genes during female flower development, differential gene expression analysis was carried out separately for female flowers and trichome tissue harvested at days 35 (Stage 1), 42 (Stage 2), 49 (Stage 3) and 56 (Stage 4) post-induction of flowering. Differentially expressed genes identified between Stage 4 and Stage 1 in flowers and trichome tissue were further categorised functionally using GO Annotation (GOA) classification in CateGOrizer (Hu et al. Online Journal of Bioinformatics. 2008, 9: 108-112). Results of CateGOrizer were further summarised and visualised in REVIGO (Supek et al. PLoS One. 2011, 6: e21800) to generate the relevant scatterplots. Selected differentially transcripts identified may be interchangeably defined by reference to UniRef100 annotation, transcript identifier and sequence identifier as shown in Table 3.

### G. Quantitative PCR Analysis

The expression of a randomly selected set of 20 differentially expressed transcripts by the RNA-Seq analysis was re-examined using qRT-PCR analysis. RNA was extracted from vegetative tissues (leaf and root) and reproductive female floral buds (Stage 1 and Stage 4) of the female strain described above. The primer sequences for the selected transcripts were designed using BatchPrimer3 (You et al. BMC Bioinformatics. 2008, 9: 253) for qRT-PCR (Table 6) with default parameters for the product size of 100 to 130 bp, GC content ranging from 40% to 60% and an optimum annealing temperature between 55 and 60° C. The F-Box gene was used as an internal reference gene. The qRT-PCR, melting curve analysis and normalisation of the obtained data against the internal control was conducted as detailed previously (Braich et al. Agronomy. 2017, 7: 53; Sudheesh et al. Int. J. Mol. Sci. 2016, 17: 1887). The correlation between the RNA-Seq and qRT-PCR data was made using Pearson's correlation coefficient.

TABLE 6
qRT-PCR primer sequences for selected transcripts

| | UniRef100 | Forward primer | Reverse primer |
|---|---|---|---|
| Transcript | Annotation | (5′->3′) | (5′->3′) |
| Cannbio_0 | UniRef100_ | GAT GGA CCC AGG | TGC GTG GCT CAG |
| 00799 | A0A2P5 AWV8 | AAG TTT CA | AAC ATA AG |
| Cannbio_0 | UniRef100_ | ACG AAC AGC CCC | GTG ATG GTG TTG |
| 13596 | A0A0B0P276 | AAT GTT AC | GCC TTC TT |
| Cannbio_0 | UniRef100_ | CAG AGA TGT CGT | TTT GGG TGA GGA |
| 33634 | A0A2P5ABA8 | CGT CCT GA | AGC TGA TT |
| Cannbio_0 | UniRef100_ | CTC TCG AGC AAG | CCA CCT TTG AAT |
| 35851 | A0A1V0QSF6 | GAA GCT TTT | TGC TTT GG |
| Cannbio_0 | UniRef100_ | AAG GGC CAC TAT | TCT GAG AAG TTC |
| 41591 | A0A2P5AWV8 | GTC AGC AC | GCC ACT CC |
| Cannbio_0 | UniRef100_ | TTT TGC CAG TTG | GTG GGT CGA CTG |
| 44988 | A7LCN2 | AAG AAG CA | GGT ATG AC |
| Cannbio_0 | UniRef100_ | TGA AGT CTT CTG | AAG AAT CTT TTT |
| 38927 | A0A059BY11 | GCT TCT TGC | GCC CGA TG |
| Cannbio_0 | UniRef100_ | CAA GGA TCG CCG | GAC CGT CCT GAA |
| 50434 | UPI000CED6849 | TTA ATG AT | CGA CAA CT |
| Cannbio_0 | UniRef100_ | CTT TGT GTT CCC | GGT TGG ACC CAA |
| 60043 | UPI000CED1C21 | CAA AGG AA | ACA AGG TA |
| Cannbio_0 | UniRef100_ | TGA CTT CAT CTT | ATC CAC TGT TTT |
| 51181 | A0A2P5FH06 | CGG CAA TG | CGG ACA CC |
| Cannbio_0 | UniRef100_ | CGT TGG TTT CGT | GCT TTG GGA CAC |
| 56951 | A0A2P5BJ37 | CCT TCA TT | ACA CTT CA |
| Cannbio_0 | UniRef100_ | AAC CCC AAA ACC | GGA CAA ACC CTC |
| 09678 | A0A142EGL4 | ACT CGT TA | AGC ATC AT |
| Cannbio_0 | UniRef100_ | GAA GAG AAG AGC | GGC AGC ATT TTT |
| 01610 | A0A2I8ANK1 | GGA GTG GA | GCT GGT AT |
| | RepID = | | |
| | A0A218ANK1_ | | |
| | IDEPO | | |
| Cannbio_0 | UniRef100_ | ACA GGC AAA CCA | AGC CAA GCT GGC |
| 42452 | Q6RXX0 | TAT GAT CG | ATA ACA CT |
| Cannbio_0 | UniRef100_ | AGC CAT GCG ATT | TCC GCT CTT CTC |
| 47069 | A0A1Q3B5J9 | TGA GAT TC | TTC CCA TA |
| Cannbio_0 | UniRef100_ | ACC AAT CCC ACT | GGG TTG AGT TGG |
| 47604 | UPI000B79458D | CTG ACG AG | CTT GTG TT |
| Cannbio_0 | UniRef100_ | CAC CAA GCT TTC | GGA GCC GAT GTT |
| 27313 | W9QMT8 | AGT GAC CA | TGA ATG AT |
| Cannbio_0 | UniRef100_ | TGG CTT CAC AGC | ACC GTT GAT CGA |
| 15972 | A0A088MFF4 | TTT TGT TG | GAT TTT GG |
| Cannbio_0 | UniRef100_ | CAA TGT GTT CAA | ATG GTT GGT CAT |
| 26293 | A0A2P5DN04 | GGC GAA TG | GTC TGC AA |
| Cannbio_0 | UniRef100_ | GGC CAA GCT TGA | TGC CAC AAA TTG |
| 63731 | A0A2P5BI20 | AAA GGT TA | AGT TTG GA |
| | F-box | CCA TTA CCA ACC | ACT GGT TCC GAA |
| | | TGA AGA AGC | CTC TGC TG |

### H. Expression Analysis of Genes Involved in Terpene and Cannabinoid Synthesis

BLASTN analysis with the threshold E-value of <10⁻¹⁰ was performed against terpene synthases and the genes involved in terpene synthesis of C. sativa (Booth et al. PLoS One. 2017, 12: e0173911) to identify the associated transcripts of interest from the current assembly. Additionally, candidate transcripts were identified as tetrahydrocannabinolic acid synthase (THCAS), cannabidiolic acid synthase-like 1 (CBDAS-like 1) and cannabidiolic acid synthase (CBDAS) based on the annotation of similarity results to UniRef100 database. The relative level of expression for these transcripts in each tissue type and across the female reproductive developmental stages was determined by normalised read count analysis. The identified candidate transcripts with normalised read count of over 100 in at least one sample were considered to be expressed significantly and were used to generate relevant heat maps with R Bioconductor packages, gplots and d3heatmap.

### Example 1

### RNA-Seq and De Novo Transcriptome Assembly

A total of seventy-one RNA-Seq libraries were sequenced aiming to obtain a minimum of 30 million reads from each sample. The transcriptome assembly was generated from a total of 6,946,497,370 sequence reads. A complete list of samples and associated details used in the de novo transcriptome assembly is provided in Table 7.

TABLE 7
Sample list and number of paired-end
reads obtained for each sample.

| Tissue Type | Read Count |
|---|---|
| Female_Flower_Stage1-Rep1 | 98,750,064 |
| Female_Flower_Stage1-Rep2 | 89,868,880 |
| Female_Flower_Stage1-Rep3 | 72,164,492 |
| Female_Flower_Stage1-Rep4 | 80,156,826 |
| Female_Flower_Stage1-Rep5 | 60,817,652 |
| Female_Flower_Stage1-Rep6 | 67,599,576 |
| Female_Flower_Stage2-Rep1 | 58,567,384 |
| Female_Flower_Stage2-Rep2 | 65,931,498 |
| Female_Flower_Stage2-Rep3 | 56,800,800 |
| Female_Flower_Stage2-Rep4 | 74,775,084 |
| Female_Flower_Stage2-Rep5 | 56,702,594 |
| Female_Flower_Stage2-Rep6 | 63,982,774 |
| Female_Flower_Stage3-Rep1 | 86,641,860 |
| Female_Flower_Stage3-Rep2 | 70,178,844 |
| Female_Flower_Stage3-Rep3 | 88,562,416 |
| Female_Flower_Stage3-Rep4 | 51,978,330 |
| Female_Flower_Stage3-Rep5 | 70,642,692 |
| Female_Flower_Stage3-Rep6 | 74,370,556 |
| Female_Flower_Stage4-Rep1 | 72,015,200 |
| Female_Flower_Stage4-Rep2 | 45,817,624 |
| Female_Flower_Stage4-Rep3 | 65,822,048 |
| Female_Flower_Stage4-Rep4 | 95,439,268 |
| Female_Flower_Stage4-Rep5 | 74,284,522 |
| Female_Flower_Stage4-Rep6 | 65,060,600 |
| Trichome_Stage1-Rep1 | 60,376,816 |
| Trichome_Stage1-Rep2 | 52,871,298 |
| Trichome_Stage1-Rep3 | 140,183,178 |
| Trichome_Stage1-Rep4 | 97,734,802 |
| Trichome_Stage1-Rep5 | 105,105,834 |
| Trichome_Stage1-Rep6 | 110,188,760 |
| Trichome_Stage2-Rep1 | 92,195,806 |
| Trichome_Stage2-Rep2 | 63,143,128 |
| Trichome_Stage2-Rep3 | 65,294,382 |
| Trichome_Stage2-Rep4 | 87,664,360 |
| Trichome_Stage2-Rep5 | 109,612,230 |
| Trichome_Stage2-Rep6 | 55,655,608 |
| Trichome_Stage3-Rep1 | 80,927,528 |
| Trichome_Stage3-Rep2 | 117,975,584 |
| Trichome_Stage3-Rep3 | 105,285,692 |
| Trichome_Stage3-Rep4 | 120,965,734 |
| Trichome_Stage3-Rep5 | 62,858,298 |
| Trichome_Stage3-Rep6 | 124,374,966 |
| Trichome_Stage4-Rep1 | 92,634,552 |
| Trichome_Stage4-Rep2 | 160,091,034 |
| Trichome_Stage4-Rep3 | 62,868,138 |
| Trichome_Stage4-Rep4 | 183,161,692 |
| Trichome_Stage4-Rep5 | 68,083,392 |
| Trichome_Stage4-Rep6 | 40,521,984 |
| Female Vegetative Leaf - fresh cutting | 34,819,074 |
| Female Vegetative Leaf - fresh cutting | 384,162,568 |
| Female Vegetative leaf - mature mother plant | 307,090,004 |
| Female Vegetative leaf - mature mother plant | 130,757,372 |
| Female Dried floral bud | 156,869,714 |
| Female Reproductive leaf | 126,705,172 |
| Female Reproductive immature bud | 126,272,602 |
| Female Mature plant reproductive bud | 161,923,146 |
| Female Immature budding plant reproductive leaf | 126,939,744 |
| Female Immature budding plant reproductive buds | 189,378,870 |
| Female Maturing plant - reproductive leaf | 51,979,970 |
| Female Maturing plant - reproductive bud | 43,600,250 |
| Female Vegetative leaf | 90,658,286 |
| Female Fresh Flower | 253,321,014 |
| Female Root-mid | 145,593,320 |
| Female Root-tip | 130,724,514 |
| Male Flower-Rep1 | 94,220,832 |
| Male Flower-Rep2 | 70,202,938 |
| Male Flower-Rep3 | 40,654,622 |
| Male Flower-Rep4 | 64,464,294 |
| Male Flower-Rep5 | 89,265,712 |
| Male Flower-Rep6 | 66,444,546 |
| Male Leaf | 93,742,426 |
| Total | 6,946,497,370 |

The high-quality trimmed reads were initially assembled using the SOAPdenovo-TRANS assembler. An empirically optimised k-mer value of 73 was used for the assembly. The statistics of the sequencing data filtering and outputs are summarised in Table 6, with the initial assembly resulting in 500,485 contigs and scaffolds with a mean size of 487 bp. Following the initial assembly, a total of 221,849 contigs were removed as they had length less than 240 bp (considerably shorter than a pair of sequence reads) and were considered likely to be spurious. A further total of 94,670 contigs were also removed, as they had less than 10 sequence reads associated with the initial assembly and their length ranged between 240-500 bp. These filtering steps removed a large number of transcripts and resulted in a total of 183,966 contigs and scaffolds remaining.

TABLE 8
Sequencing outputs and transcriptome assembly statistics
of the primary, secondary and filtered assembly.

| | |
|---|---|
| Assembly | Statistics |

Primary Assembly: SOAPdenovo-Trans

| Total number of transcripts | 500,485 | |
|---|---|---|
| Total base pairs (without N) | 241,253,446 | bp |
| N50 length | 954 | bp |

Secondary Assembly: CAP3

| Number of transcripts | 143,671 | |
|---|---|---|
| Total base pairs | 104,880,973 | bp |
| N50 | 1071 | bp |

Final Assembly: Filtered

| Number of transcripts | 64,727 | |
|---|---|---|
| Total base pairs | 57,300,518 | bp |
| N50 | 1846 | bp |

The initially assembled scaffolds (57,268) that were identified as fork, bubble and complex loci in nature from the SOAPdenovo-TRANS assembly were individually assembled using CAP3. The CAP3 assembler resolved 24,840 scaffolds relating to 7,143 loci (each representing a single sequence in the transcriptome assembly). The majority of scaffolds that were not resolved by the CAP3 assembly step, were complex loci (78.9%). The unresolved scaffolds (32,428) were analysed, and a single longest transcript for each locus from these scaffolds was retained in the assembly, this added another 9,830 transcripts to the assembly. The secondary enhanced assembly (Table 7) resulted in 143,671 contigs and scaffolds with N50 of 1071 bp and N90 of 287 bp with the largest transcript length of 167,637 bp.

### Example 2

### Classification and Annotation of the Transcriptome Assembly

The secondary assembly was used as the query file for a BLASTX search against UniRef100 database and identified 82,610 transcripts corresponding to 53,652 unique UniRef100 identifiers. Contigs and scaffolds that were not annotated by UniRef100 BLASTX search were removed from the transcriptome assembly. Based on the obtained annotation of the UniRef100 protein, a total of 19,440 transcripts exhibited the highest matches to sequences of non-plant derived sources. A small proportion of these non-plant identified transcripts (1,557) showed high-value matches of moderate similarity to the published cannabis transcriptome assemblies of PK and Finola (van Bakal supra) and were therefore retained in the assembly, all other non-plant identified sequences were removed from the assembly. Out of the 61,061 unannotated sequences, 36,392 transcripts displayed similarity matches to either or both PK and Finola transcriptome assemblies but were not included for further analysis as they failed to return a match to a known protein. The final filtered transcriptome assembly comprised of 64,727 contigs and scaffolds (Table 7). The size distribution of the final transcriptome assembly was determined (FIG. 5). The majority of the contigs and scaffolds ranged between 240-300 bp in length (42.2%), followed by those that were above the length of 2000 bp (12.3%) with the largest transcript length of 107,602 bp and N50 of 1,847 bp.

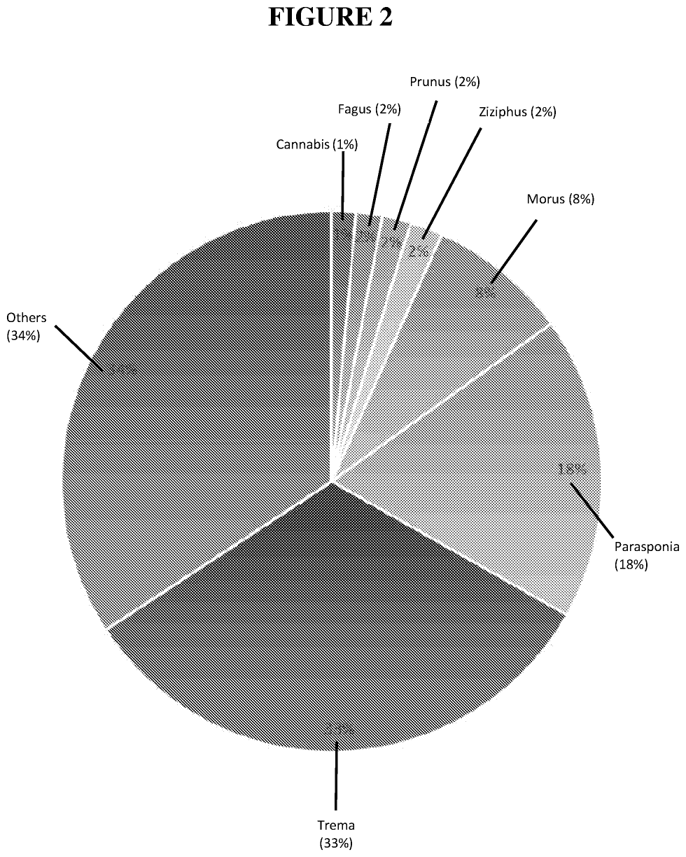

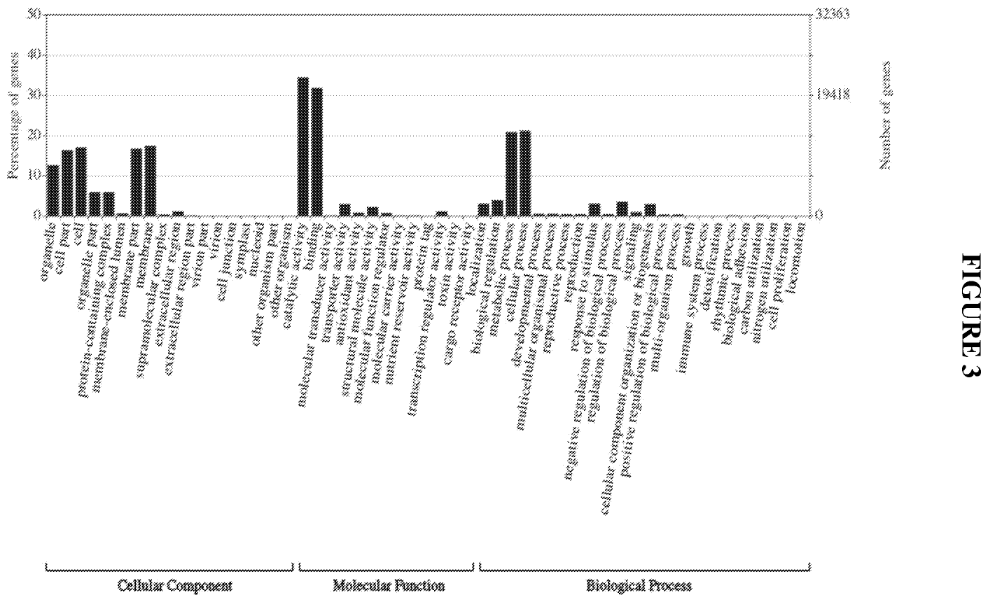

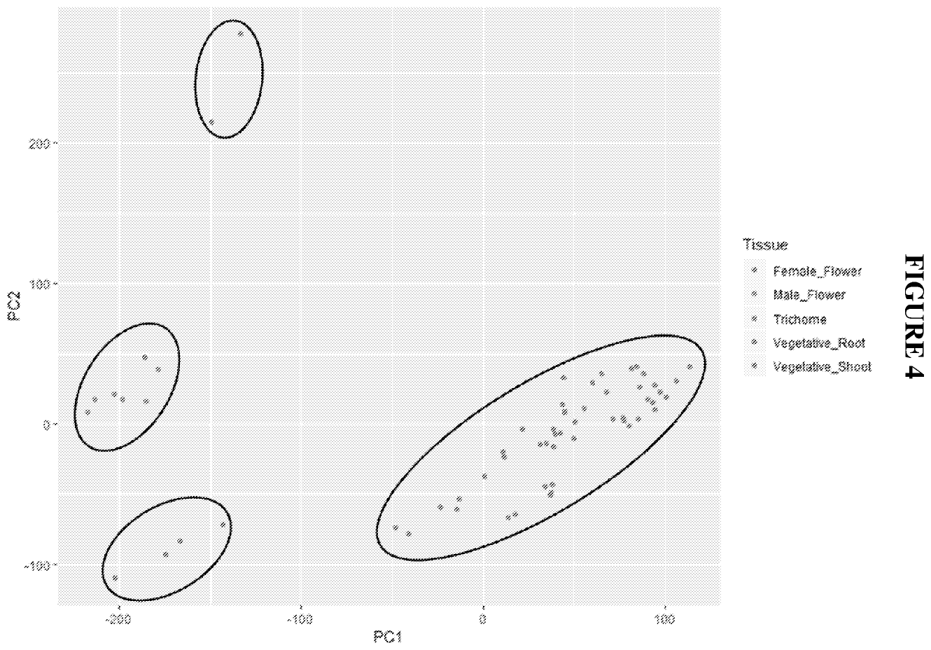

The BLASTX analysis to the UniRef100 database also revealed the distribution of similarity of the assembled transcripts to other plant species. FIG. 2 represents the genus wide similarity distribution of the transcripts from the current study that was obtained from the taxonomy of the corresponding similar protein. A total of 21,012 transcripts displayed the highest similarity to Trema orientalis, followed by Parasponia andersonii (11,721) and Morus notabilis (5,363).

Comparison of the final transcriptome assembly to the previously published cannabis transcriptome and CDS datasets revealed that the current assembly captured 89% of the transcripts of PK (van Bakal, supra), 93.7% transcripts of Finola (van Bakal, supra) and 78.7% of the coding sequences (CDS) of the CBDrx assembly (Grassa et al. supra). A total of 48,893 of the assembly transcripts were present in all three datasets, while 2,726 of the contigs and scaffolds were found to be exclusive to the assembly and have not been previously characterised in this species' datasets.

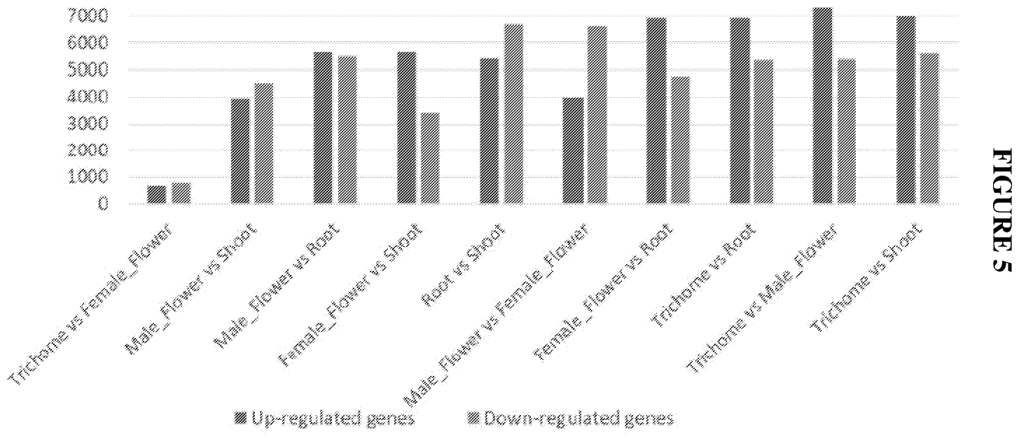

Gene function categories of the contigs and scaffolds generated from the current transcriptome assembly were obtained by assigning GO terms based on the sequence similarity to UniRef100 database. A total of 41,457 transcripts from the assembly were assigned at least one GO term (FIG. 3). GO assignment was recorded to be the highest for molecular function (47.3%), followed by cellular component (27.8%) and biological process (25%). Amongst the annotated sequences, molecular function categories included catalytic activity (22,272), binding (20,593), transporter activity (1,881), structural molecule activity (1,406) and other categories (1,851). Cellular component categories included membrane (11,250), cell (11,019), membrane part (10,789), cell part (10,578), organelle (8,176) and other categories (9,082). In addition, biological process categories were comprised of cellular process (13,640), metabolic process (13,447), biological regulation (2,546), regulation of biological process (2,288), localisation (1,926), response to stimulus (1,911), cellular component organisation or biogenesis (1,884) and other categories (2,545).

### Example 3

### Differential Gene Expression Analysis

Following normalisation of read counts, similarity between samples of various tissue-types was assessed by plotting a principal component analysis (PCA) graph from the normalised count data (FIG. 4). Normalised data from read counts obtained from each tissue type formed four distinct clusters of root tissues, shoot tissues (with one outlier), female floral and male floral tissues.

Comparisons of gene expression were made between the distinct tissue types to identify differentially expressed genes as represented in FIG. 5. Comparisons between trichome and female flower tissue revealed the least divergence in gene expression with only 1,479 differentially expressed genes (46.4% up-regulated and 53.6% down-regulated genes) in trichomes when compared to female flowers with log 2Fold Change ranging from −14.9 to 6.2. Female floral tissues, especially the trichomes were found to be the most distinct group due to the maximum divergence from all other tissue types.

Differentially Expressed Genes Associated with Sex Determination

A total of 12,669, 12,598 and 12,277 differentially expressed genes were found in trichomes as compared to male flower, vegetative shoot and root tissues respectively. Glycoside hydrolase, naringenin-chalcone synthase, lipoxygenase and sieve element occlusion genes were the most frequently found gene nomenclature that was up-regulated in trichomes. Comparisons between female and male reproductive floral tissues identified genes that were most commonly up-regulated genes in male flowers annotated as leucine-rich repeat (LRR) and F-box domain containing proteins, pseudo-autosomal region (PAR) and endonucleases. A summary of upregulated genes with their annotations based on UniRef100 database similarity results and log 2Fold Change value for male and reproductive tissues are detailed in Tables 9 and 10.

These results were further refined by comparing the expression of female trichome gene expression with male flower tissue to identify a subset of transcripts that are significantly differentially expressed between female and male cannabis plants, as detailed in Table 9. Lipoxygenase, cannabinoid synthesis protein, geranyl diphosphate pathway protein, MEP pathway protein, terpene synthesis protein, MADs box floral initiation transtriction factor protein were significantly unregulated in female cannabis plants as compared to male cannabis plants. Additionally, common cannabis allergens and LRR containing proteins were significantly down-regulated in female cannabis plants as compared to male cannabis plants.

These data enable methods for determining the sex of a cannabis plant. In particular, the differential expression of genes encoding lipoxygenes, cannabinoid synthesis protein, geranyl diphospohate pathway protein, MEP pathway protein, terpene synthesis protein, MADs box floral initiation transcription factor, cannabis allergens and LRR containing protein can be used to determine the sex of a cannabis plant.

TABLE 9
Upregulated genes identified in male reproductive tissue with log2FoldChange and UniRef100 annotation.

| | | | log2 |
|---|---|---|---|
| UniRef100 ID | Name | Taxonomy | FoldChange |
| UniRef100_A0A2P5A8J8 | LRR domain containing protein | Parasponia | 2.584311369 |
| | | andersonii | |
| UniRef100_A0A2P5A8M0 | LRR domain containing protein | Parasponia | 10.75105612 |
| | | andersonii | |
| UniRef100_A0A2P4J0F4 | Leucine-rich repeat receptor protein kinase | Quercus suber | 2.653176087 |
| UniRef100_A0A2P5AAY3 | Leucine-rich repeat domain containing protein | Parasponia | 2.372007564 |
| | | andersonii | |
| UniRef100_A0A2P5AD11 | NB-ARC domain, LRR domain containing | Parasponia | 6.340204972 |
| | protein | andersonii | |
| UniRef100_A0A2P5AJ53 | Leucine-rich repeat domain containing protein | Parasponia | 2.819412319 |
| | | andersonii | |
| UniRef100_A0A2P5APQ0 | LRR domain containing protein | Parasponia | 2.043557817 |
| | | andersonii | |
| UniRef100_A0A2P5AQP6 | LRR domain containing protein | Trema | 3.087498068 |
| | | orientalis | |
| UniRef100_A0A2P5AS06 | TIR-NBS-LRR-like protein | Trema | 5.21299362 |
| | | orientalis | |
| UniRef100_A0A2P5AZK2 | Leucine-rich repeat, cysteine-containing subtype | Parasponia | 5.067554302 |
| | | andersonii | |
| UniRef100_A0A2P5BH40 | LRR domain containing protein | Parasponia | 4.565495491 |
| | | andersonii | |
| UniRef100_A0A2P5C7N5 | LRR domain containing protein (Fragment) | Trema | 3.051025208 |
| | | orientalis | |
| UniRef100_A0A2P5CDW4 | LRR domain containing protein | Parasponia | 2.028530518 |
| | | andersonii | |
| UniRef100_A0A2P5CG16 | LRR domain containing protein | Trema | 2.432615201 |
| | | orientalis | |
| UniRef100_A0A2P5CGL7 | NB-ARC domain, LRR domain containing | Trema | 8.165804904 |
| | protein | orientalis | |
| UniRef100_A0A2P5EEJ9 | LRR domain containing protein | Trema | 6.473433703 |
| | | orientalis | |
| UniRef100_A0A2P5EEK9 | NB-ARC domain, LRR domain containing | Trema | 4.244387328 |
| | protein | orientalis | |
| UniRef100_A0A2P5EQT3 | LRR domain containing protein (Fragment) | Trema | 6.800527402 |
| | | orientalis | |
| UniRef100_A0A2P5EQU2 | LRR domain containing protein (Fragment) | Trema | 2.283153319 |
| | | orientalis | |
| UniRef100_A0A2P5EQU3 | LRR domain containing protein | Trema | 12.47888288 |
| | | orientalis | |
| UniRef100_W9SL85 | Putative leucine-rich repeat receptor-like protein | Mortis notabilis | 8.223396603 |
| | kinase | | |
| UniRef100_W9SBZ2 | LRR receptor-like serine/threonine-protein kinase | Mortis notabilis | 2.903008959 |
| | ERL1 | | |
| UniRef100_W9QPK0 | Putative LRR receptor-like serine/threonine- | Mortis notabilis | 4.297318091 |
| | protein kinase | | |
| UniRef100_UPI000C04C0D2 | Leucine-rich repeat receptor-like protein kinase | Durio | 4.480570386 |
| | TDR | zibethinus | |
| UniRef100_UPI000C1CEE78 | Leucine-rich repeat extensin-like protein 3 | Olea europaea | 5.073465374 |
| | | var. sylvestris | |
| UniRef100_UPI000C1D2EC7 | F-box/LRR-repeat protein At3g58900-like | Olea europaea | 5.959724397 |
| | isoform X3 | var. sylvestris | |
| UniRef100_UPI000B7B2303 | Leucine-rich repeat protein 1-like | Prunus avium | 5.122303618 |
| UniRef100_UPI00098E1879 | F-box/LRR-repeat protein 20-like | Asparagus | 5.064985847 |
| | | officinalis | |
| UniRef100_UPI0009E1E0E4 | LRR receptor-like serine/threonine-protein kinase | Phalaenopsis | 2.837323531 |
| | ERL2 | equestris | |
| UniRef100_UPI00077E47B6 | Leucine-rich repeat receptor protein kinase EMS1 | Ziziphus jujuba | 2.851919997 |
| UniRef100_A0A2P6QR86 | Putative P-loop containing nucleoside | Rosa chinensis | 2.034860714 |
| | triphosphate hydrolase, leucine-rich repeat | | |
| | domain, L | | |
| UniRef100_A0A2P5FXI6 | NB-ARC domain, LRR domain containing | Trema | 4.899768085 |
| | protein | orientalis | |
| UniRef100_A0A2P5FYW2 | LRR domain containing protein | Trema | 2.298555746 |
| | | orientalis | |
| UniRef100_A0A2P5FGL8 | LRR domain containing protein | Trema | 2.027433617 |
| | | orientalis | |
| UniRef100_A0A2P5EX22 | LRR domain containing protein | Trema | 3.075711442 |
| | | orientalis | |
| UniRef100_A0A2P5EQ66 | NB-ARC domain, LRR domain containing | Trema | 2.719532002 |
| | protein | orientalis | |
| UniRef100_A0A2P5ERE1 | LRR domain containing protein | Trema | 2.655186359 |
| | | orientalis | |
| UniRef100_A0A2P5EH55 | NB-ARC domain, LRR domain containing | Trema | 2.900051921 |
| | protein | orientalis | |
| UniRef100_A0A2P5E282 | LRR domain containing protein | Parasponia | 3.233553191 |
| | | andersonii | |
| UniRef100_A0A2P5DJ02 | LRR domain containing protein | Parasponia | 3.582878227 |
| | | andersonii | |
| UniRef100_A0A2P5DE20 | LRR domain containing protein | Parasponia | 2.571448582 |
| | | andersonii | |
| UniRef100_A0A2P5D5U0 | LRR domain containing protein | Trema | 2.624405874 |
| | | orientalis | |
| UniRef100_A0A2P5D2A4 | TIR-NBS-LRR-like protein | Parasponia | 5.396322118 |
| | | andersonii | |
| UniRef100_A0A2P5D2X8 | NB-ARC domain, LRR domain containing | Parasponia | 3.723680229 |
| | protein | andersonii | |
| UniRef100_A0A2P5D159 | LRR domain containing protein | Parasponia | 3.085203603 |
| | | andersonii | |
| UniRef100_A0A2P5CLZ3 | LRR domain containing protein | Parasponia | 4.166360758 |
| | | andersonii | |
| UniRef100_A0A2P5CN81 | LRR domain containing protein | Parasponia | 2.010360124 |
| | | andersonii | |
| UniRef100_A0A2P5B818 | F-box domain containing protein | Trema | 7.733782705 |
| | | orientalis | |
| UniRef100_A0A2P5C1E8 | F-box domain containing protein | Trema | 5.778501048 |
| | | orientalis | |
| UniRef100_A0A2P5C2W0 | F-box domain containing protein | Parasponia | 6A48723797 |
| | | andersonii | |
| UniRef100_A0A2P5CCR9 | F-box domain containing protein | Trema | 3.997690841 |
| | | orientalis | |
| UniRef100_A0A2P5CSY1 | F-box domain containing protein | Parasponia | 3.497846441 |
| | | andersonii | |
| UniRef100_A0A2P5CUS4 | F-box domain containing protein | Trema | 4.854472339 |
| | | orientalis | |
| UniRef100_A0A2P5CZK5 | F-box domain containing protein | Trema | 2.258513449 |
| | | orientalis | |
| UniRef100_A0A2P5D357 | F-box domain containing protein | Parasponia | 2.970187933 |
| | | andersonii | |
| UniRef100_A0A2P5D5U1 | F-box domain containing protein | Trema | 5.535942803 |
| | | orientalis | |
| UniRef100_A0A2P5D5W8 | F-box domain containing protein | Trema | 5.167658359 |
| | | orientalis | |
| UniRef100_A0A2P5D7L6 | F-box domain containing protein | Parasponia | 5.832750359 |
| | | andersonii | |
| UniRef100_A0A2P5D7L9 | F-box domain containing protein | Parasponia | 2.133473967 |
| | | andersonii | |
| UniRef100_A0A2P5DAG9 | F-box domain containing protein | Parasponia | 6.413277644 |
| | | andersonii | |
| UniRef100_A0A2P5EA83 | F-box domain containing protein | Trema | 2.224986711 |
| | | orientalis | |
| UniRef100_A0A2P5EAR9 | F-box domain containing protein | Trema | 5.132100732 |
| | | orientalis | |
| UniRef100_A0A2P5EBG9 | F-box domain containing protein | Trema | 2.339183571 |
| | | orientalis | |
| UniRef100_A0A2P5EDF2 | F-box domain containing protein | Trema | 2.262631836 |
| | | orientalis | |
| UniRef100_A0A2P5ER64 | F-box domain containing protein | Trema | 6.202222969 |
| | | orientalis | |
| UniRef100_A0A2P5EYU8 | F-box domain containing protein | Trema | 2.839942073 |
| | | orientalis | |
| UniRef100_A0A2P5F0Q7 | F-box domain containing protein | Trema | 5.947877395 |
| | | orientalis | |
| UniRef100_A0A2P5F918 | F-box domain containing protein | Trema | 6.185395978 |
| | | orientalis | |
| UniRef100_A0A2P5FC09 | F-box domain containing protein (Fragment) | Trema | 4.227972396 |
| | | orientalis | |
| UniRef100_A0A2P5FC48 | F-box domain containing protein | Trema | 6.534610927 |
| | | orientalis | |
| UniRef100_A0A2P5FD59 | F-box domain containing protein | Trema | 3.902672368 |
| | | orientalis | |
| UniRef100_A0A2P5FFC2 | F-box domain containing protein | Trema | 3.787581334 |
| | | orientalis | |
| UniRef100_A0A2P5FGL8 | ERR domain containing protein | Trema | 2.027433617 |
| | | orientalis | |
| UniRef100_A0A2P5FTA0 | F-box domain containing protein | Trema | 7.019657046 |
| | | orientalis | |
| UniRef100_UPI000B78B766 | F-box protein At5g07610-like | Hevea | 2.820087687 |
| | | brasiliensis | |
| UniRef100_UPI000CE2776C | F-box protein At3g07870-like | Quercus suber | 5.972916709 |
| UniRef100_UPI000CED1855 | Probable F-box protein At3g61730 | Morus notabilis | 5.380472088 |
| UniRef100_W9QCJ9 | F-box protein | Morus notabilis | 4.180885148 |
| UniRef100_W9RWX6 | Putative F-box protein | Morus notabilis | 5.063927808 |
| UniRef100_W9S0E9 | F-box/kelch-repeat protein | Morus notabilis | 5.353335309 |
| UniRef100_W9S804 | F-box/kelch-repeat protein SKIP6 | Morus notabilis | 2.654883434 |
| UniRef100_A0A2P5CA60 | Endonuclease/exonuclease/phosphatase | Parasponia | 7.239099324 |
| | | andersonii | |
| UniRef100_A0A2P5FTA7 | AP endonuclease 1, binding site (Fragment) | Trema | 5.986481868 |
| | | orientalis | |
| UniRef100_A0A2P5G1V5 | Endonuclease/exonuclease/phosphatase | Trema | 5.329957774 |
| | | orientalis | |

TABLE 10
Upregulated genes identified in female reproductive tissue with log2FoldChange and UniRef100 annotation.

| | | | log2 Fold |
|---|---|---|---|
| UniRef100 Annotation | Name | Taxonomy | Change |
| UniRef100_A0A059AAV9 | Lipoxygenase | Eucalyptus | 4.93827313 |
| | | grandis | |
| UniRef100_A0A061F9Z0 | Lipoxygenase | Theobroma | 4.175337767 |
| | | cacao | |
| UniRef100_A0A061FIC3 | Glyceraldehyde-3-phosphate dehydrogenase | Theobroma | 2.156346813 |
| | (Fragment) | cacao | |
| UniRef100_A0A0B4SX31 | Ribulose bisphosphate carboxylase large chain | Cannabis sativa | 3.641518888 |
| | (Fragment) | | |
| UniRef100_A0A0D2VLQ6 | Non-specific lipid-transfer protein | Gossypium | 3.078706622 |
| | | raimondii | |
| UniRef100_A0A0E0MLJ2 | Ribulose bisphosphate carboxylase small chain | Oryza punctata | 5.742449845 |
| UniRef100_A0A118JUA2 | Lipoxygenase (Fragment) | Cynara | 5.185977505 |
| | | cardunculus var. | |
| | | scolymus | |
| UniRef100_A0A126WZD3 | Putative LOV domain-containing protein | Cannabis sativa | 2.632302795 |
| UniRef100_A0A1D1YHK1 | Glyceraldehyde-3-phosphate dehydrogenase | Anthurium | 2.057355665 |
| | | amnicola | |
| UniRef100_A0A1R3HLX0 | Glycoside hydrolase | Corchorus | 2.711547367 |
| | | capsularis | |
| UniRef100_A0A1R3KR48 | Glycoside hydrolase, family 19, catalytic | Corchorus | 3.099498523 |
| | | olitorius | |
| UniRef100_A0A200PXQ4 | Glycoside hydrolase | Macleaya | 3.218002443 |
| | | cordata | |
| UniRef100_A0A220QME3 | Lipoxygenase (Fragment) | Solanum | 2.8878376 |
| | | muricatum | |
| UniRef100_A0A251S466 | Non-specific lipid-transfer protein | Helianthus | 3.238413166 |
| | | annuus | |
| UniRef100_A0A2C9VH29 | Non-specific lipid-transfer protein | Manihot | 2.394328177 |
| | | esculenta | |
| UniRef100_A0A2I0IAB4 | Non-specific lipid-transfer protein | Punica granatum | 7.541378905 |
| UniRef100_A0A2I4EBW6 | Ribulose bisphosphate carboxylase small chain | Juglans regia | 5.625321102 |
| UniRef100_A0A2I4FKH6 | Ribulose bisphosphate carboxylase/oxygenase | Juglans regia | 3.909401258 |
| | activase 2, chloroplastic-like | | |
| UniRef100_A0A2K3LAF4 | Linoleate 13s-lipoxygenase 2-1 chloroplastic- | Trifolium | 3.355655157 |
| | like (Fragment) | pratense | |
| UniRef100_A0A2K3N6Y1 | Ribulose bisphosphate carboxylase/oxygenase | Trifolium | 2.110419083 |
| | activase (Fragment) | pratense | |
| UniRef100_A0A2P4GL99 | Linoleate 13s-lipoxygenase 2-1, chloroplastic | Quercus suber | 3.025041434 |
| UniRef100_A0A2P5ASL2 | Glycoside hydrolase | Parasponia | 6.088503128 |
| | | andersonii | |
| UniRef100_A0A2P5AX40 | Lipoxygenase | Parasponia | 4.877064298 |
| | | andersonii | |
| UniRef100_A0A2P5AX41 | Glycoside hydrolase | Parasponia | 5.82645551 |
| | | andersonii | |
| UniRef100_A0A2P5AX77 | Lipoxygenase | Parasponia | 5.315568521 |
| | | andersonii | |
| UniRef100_A0A2P5BBD1 | Glycoside hydrolase | Parasponia | 4.166554862 |
| | | andersonii | |
| UniRef100_A0A2P5BS17 | Non-specific lipid-transfer protein | Parasponia | 6.037163272 |
| | | andersonii | |
| UniRef100_A0A2P5BS28 | Non-specific lipid-transfer protein | Parasponia | 4.160373582 |
| | | andersonii | |
| UniRef100_A0A2P5BS46 | Non-specific lipid-transfer protein | Parasponia | 6.919556148 |
| | | andersonii | |
| UniRef100_A0A2P5BS89 | Non-specific lipid-transfer protein | Parasponia | 8.068327973 |
| | | andersonii | |
| UniRef100_A0A2P5BSA4 | Lipoxygenase | Trema orientalis | 7.035287454 |
| UniRef100_A0A2P5BSD7 | Lipoxygenase | Trema orientalis | 2.935348795 |
| UniRef100_A0A2P5BSG5 | Glycoside hydrolase | Trema orientalis | 7.480728901 |
| UniRef100_A0A2P5BWS3 | Sieve element occlusion | Parasponia | 5.969376574 |
| | | andersonii | |
| UniRef100_A0A2P5BX18 | Sieve element occlusion | Trema orientalis | 4.884879138 |
| UniRef100_A0A2P5C3Q8 | Glycoside hydrolase | Parasponia | 6.727610933 |
| | | andersonii | |
| UniRef100_A0A2P5C5P6 | Glycoside hydrolase (Fragment) | Parasponia | 3.144243168 |
| | | andersonii | |
| UniRef100_A0A2P5CBA7 | Phosphoglycerate kinase | Trema orientalis | 4.797074251 |
| UniRef100_A0A2P5CFQ0 | Lipoxygenase | Trema orientalis | 4.175994389 |
| UniRef100_A0A2P5CFQ0 | Lipoxygenase | Trema orientalis | 2.597211603 |
| UniRef100_A0A2P5CFQ2 | Lipoxygenase | Trema orientalis | 4.67020365 |
| UniRef100_A0A2P5CLL1 | Glycoside hydrolase | Parasponia | 3.733469994 |
| | | andersonii | |
| UniRef100_A0A2P5CYN2 | Non-specific lipid-transfer protein | Trema orientalis | 2.557730306 |
| UniRef100_A0A2P5D0H1 | Glycoside hydrolase | Trema orientalis | 6.65575592 |
| UniRef100_A0A2P5D8V3 | Lipoxygenase | Parasponia | 2.428938254 |
| | | andersonii | |
| UniRef100_A0A2P5D8W1 | Glycoside hydrolase | Parasponia | 6.229844533 |
| | | andersonii | |
| UniRef100_A0A2P5D8Y7 | Lipoxygenase | Parasponia | 7.985886484 |
| | | andersonii | |
| UniRef100_A0A2P5D8Z0 | Lipoxygenase | Parasponia | 2.183284388 |
| | | andersonii | |
| UniRef100_A0A2P5DAB0 | Glycosyltransferase | Trema orientalis | 8.450460661 |
| UniRef100_A0A2P5DBE1 | Glycoside hydrolase | Parasponia | 6.594209356 |
| | | andersonii | |
| UniRef100_A0A2P5DT08 | Lipoxygenase | Parasponia | 3.407587262 |
| | | andersonii | |
| UniRef100_A0A2P5DWF8 | Heat shock protein 70 family | Trema orientalis | 7.961594156 |
| UniRef100_A0A2P5DWQ6 | Glycoside hydrolase | Parasponia | 3.265394823 |
| | | andersonii | |
| UniRef100_A0A2P5DWV1 | Heat shock protein 70 family | Parasponia | 4.946089591 |
| | | andersonii | |
| UniRef100_A0A2P5DWX9 | Heat shock protein 70 family | Parasponia | 5.209357616 |
| | | andersonii | |
| UniRef100_A0A2P5DXU2 | Sieve element occlusion | Trema orientalis | 9.283226678 |
| UniRef100_A0A2P5DZ03 | Glycoside hydrolase | Parasponia | 4.745835586 |
| | | andersonii | |
| UniRef100_A0A2P5E6N2 | Glycoside hydrolase | Trema orientalis | 5.001442051 |
| UniRef100_A0A2P5E6Z2 | Lipoxygenase | Trema orientalis | 6.889235713 |
| UniRef100_A0A2P5E6Z8 | Lipoxygenase | Trema orientalis | 4.145962542 |
| UniRef100_A0A2P5E723 | Lipoxygenase | Trema orientalis | 6.558193101 |
| UniRef100_A0A2P5E8Y8 | Glycoside hydrolase | Trema orientalis | 2.286576913 |
| UniRef100_A0A2P5EBE3 | Glycoside hydrolase | Trema orientalis | 4.590442436 |
| UniRef100_A0A2P5EQA8 | Glycoside hydrolase | Trema orientalis | 3.712197855 |
| UniRef100_A0A2P5EQB9 | Glycoside hydrolase | Trema orientalis | 4.044789463 |
| UniRef100_A0A2P5EV07 | Heat shock protein 70 family | Trema orientalis | 5.263461916 |
| UniRef100_A0A2P5F1R0 | Glycoside hydrolase | Trema orientalis | 7.181057213 |
| UniRef100_A0A2P5F2I0 | Heat shock protein 70 family | Trema orientalis | 3.221039957 |
| UniRef100_A0A2P5F4K8 | Non-specific lipid-transfer protein | Trema orientalis | 5.049910858 |
| UniRef100_A0A2P5F612 | Glycoside hydrolase (Fragment) | Trema orientalis | 4.187068143 |
| UniRef100_A0A2P5FMH8 | Sieve element occlusion | Trema orientalis | 7.357185525 |
| UniRef100_A0A2P5FQM2 | Glycoside hydrolase | Trema orientalis | 2.654573604 |
| UniRef100_A0A2P5FW38 | Glycoside hydrolase | Trema orientalis | 3.841840318 |
| UniRef100_A0A2P5FWP7 | Lipoxygenase | Trema orientalis | 2.662901556 |
| UniRef100_A0A2P5RSK3 | Non-specific lipid-transfer protein | Gossypium | 3.432958946 |
| | | barbadense | |
| UniRef100_B6CQU6 | Non-specific lipid-transfer protein | Prunus dulcis × | 6.297101551 |
| | | Prunus persic | |
| UniRef100_B9V185 | Naringenin-chalcone synthase | Humulus lupulus | 5.199165477 |
| UniRef100_C4NZX3 | Lipoxygenase | Camellia sinensis | 3.659901689 |
| UniRef100_D4QD74 | Non-specific lipid-transfer protein | Dianthus | 5.070863499 |
| | | caryophyllu | |
| UniRef100_D4QD75 | Non-specific lipid-transfer protein | Dianthus | 7.421832096 |
| | | caryophyllu | |
| UniRef100_D5FUD8 | Lipoxygenase | Vitis vinifera | 5.710677902 |
| UniRef100_G7J632 | Lipoxygenase | Medicago | 2.233472348 |
| | | truncatula | |
| UniRef100_I6QLEI | Non-specific lipid-transfer protein | Vitis | 7.208953546 |
| | | pseudoreticulata | |
| UniRef100_I6XT51 | Betvl-like protein | Cannabis sativa | 6.574745719 |
| UniRef100_022077 | Ribulose bisphosphate carboxylase small chain, | Fagus crenata | 7.8472185 |
| | chloroplastic | | |
| UniRef100_P85894 | Non-specific lipid-transfer protein | Morus nigra | 7.996654482 |
| UniRef100_Q8RVK9 | Naringenin-chalcone synthase | Cannabis sativa | 7.465255392 |
| UniRef100_Q9FV19 | Heat shock protein 70 (Fragment) | Cucurbita pepo | 5.89601333 |
| UniRef100_S4TIK6 | Non-specific lipid-transfer protein | Gossypium | 7.540238185 |
| | | hirsutum | |
| UniRef100_UPI0002C2FCCF | Non-specific lipid-transfer protein 3-like | Fragaria vesca | 7.647183042 |
| | | subsp. vesca | |
| UniRef100_UPI00057A08AD | Linoleate 13S-lipoxygenase 2-1, chloroplastic- | Populus | 8.2485087 |
| | like | euphratica | |
| UniRef100_UPI00077E9C90 | Non-specific lipid-transfer protein 1-like | Ziziphus jujuba | 7.459418267 |
| UniRef100_UPI00077EB0B4 | SIEVE ELEMENT OCCLUSION B-like | Ziziphus jujuba | 4.333218171 |
| UniRef100_UPI000786A395 | Linoleate 13S-lipoxygenase 2-1, chloroplastic | Arachis | 4.164387209 |
| | | duranensis | |
| UniRef100_UPI0008488CCA | Probable linoleate 9S-lipoxygenase 5 | Theobroma | 4.33199754 |
| | | cacao | |
| UniRef100_UPI00085AA86D | Phosphoglycerate kinase, cytosolic-like | Raphanus sativus | 2.047880107 |
| UniRef100_UPI000B7B928A | protein SIEVE ELEMENT OCCLUSION B-like | Prunus avium | 4.354471094 |
| UniRef100_UPI000B8CF377 | Ribulose bisphosphate carboxylase/oxygenase | Carica papaya | 2.989959642 |
| | activase, chloroplastic-like | | |
| UniRef100_UPI000CED6B2E | Non-specific lipid-transfer protein 1 | Morus notabilis | 8.521801257 |
| UniRef100_V4U560 | Lipoxygenase | Citrus Clementina | 2.991466517 |
| UniRef100_W9R031 | Lipoxygenase | Morus notabilis | 3.255123443 |

TABLE 11
Significantly differentially expressed transcripts between female and male cannabis plants.

| | | Trichome v |
|---|---|---|
| Transcript ID | Gene Product | M_Flower (log₂FC) |
| Cannbio_002094 | Lipoxygenase | 3.232853616 |
| Cannbio_002844 | Lipoxygenase | 5.918017984 |
| Cannbio_015133 | Lipoxygenase | 5.104404518 |
| Cannbio_017395 | Lipoxygenase | 4.781219806 |
| Cannbio_019068 | Lipoxygenase | 6.057977958 |
| Cannbio_024416 | Lipoxygenase | 5.444136728 |
| Cannbio_025677 | Lipoxygenase | 6.088253071 |
| Cannbio_027834 | Lipoxygenase | 4.32198756 |
| Cannbio_028191 | Lipoxygenase | 2.735881293 |
| Cannbio_028346 | Lipoxygenase | 5.564597559 |
| Cannbio_032734 | Lipoxygenase | 5.758202997 |
| Cannbio_032880 | Lipoxygenase | 5.619729191 |
| Cannbio_038873 | Lipoxygenase | 2.212030735 |
| Cannbio_046768 | Lipoxygenase | 3.318156989 |
| Cannbio_046769 | Lipoxygenase | 7.024617171 |
| Cannbio_018948 | Lipoxygenase | 6.007390611 |
| Cannbio_035255 | Lipoxygenase | 2.233472348 |
| Cannbio_036789 | Lipoxygenase | 2.780368914 |
| Cannbio_040003 | Linoleate 13s-lipoxygenase 2-1, chloroplastic | 3.025041434 |
| Cannbio_040162 | Lipoxygenase | 2.794819976 |
| Cannbio_041476 | Lipoxygenase | 6.133852492 |
| Cannbio_036283 | Lipoxygenase | 3.257713806 |
| Cannbio_041619 | Lipoxygenase | 4.59769289 |
| Cannbio_041633 | Lipoxygenase | 7.03556302 |
| Cannbio_042000 | Lipoxygenase | 4.683276187 |
| Cannbio_001307 | Allergen Ole e 1, conserved site | 2.425921149 |
| Cannbio_010274 | Bet v I type allergen | −3.096781988 |
| Cannbio_010479 | Pollen allergen Ole e 1 family | −4.517842961 |
| Cannbio_013018 | Ves allergen | −5.009531694 |
| Cannbio_013730 | Major pollen allergen Lol pI | −3.529512316 |
| Cannbio_013884 | Major pollen allergen | −2.334480684 |
| Cannbio_014187 | Major pollen allergen Lol pI | −4.237799375 |
| Cannbio_014514 | Pollen Ole e 1 allergen and extensin family protein | −4.057143618 |
| Cannbio_018080 | Bet v I type allergen | −3.396439795 |
| Cannbio_021476 | Major pollen allergen Lol pI | −3.089774719 |
| Cannbio_024692 | Cysteine-rich secretory protein, allergen V5/Tpx-1-related | −6.761868652 |
| Cannbio_027848 | Pollen Ole e 1 allergen and extensin family protein | −4.654018448 |
| Cannbio_039084 | Pollen Ole e 1 allergen and extensin family protein | −4.445252947 |
| Cannbio_046671 | Major pollen allergen Lol pI | −4.756709909 |
| Cannbio_048504 | Pollen allergen ole e | −5.236800559 |
| Cannbio_048042 | Allergen Ole e 1, conserved site | 6.058452404 |
| Cannbio_058401 | Pollen Ole e I family allergen protein | −4.518993782 |
| Cannbio_058961 | Ves allergen | −2.496282142 |
| Cannbio_061193 | Lipid transfer protein/Par allergen | −5.447020123 |
| Cannbio_064056 | Major pollen allergen Lol pI | −3.443216488 |
| Cannbio_008463 | TIR-NBS-LRR-like protein | −3.296824116 |
| Cannbio_022325 | TIR-NBS-LRR-like protein | −4.972130408 |
| Cannbio_000445 | TIR-NBS-LRR-like protein | 2.383381609 |
| Cannbio_015748 | TIR-NBS-LRR-like protein | 3.2316348 |
| Cannbio_042929 | TIR-NBS-LRR-like protein | −5.275680552 |
| Cannbio_052701 | TIR-NBS-LRR-like protein | −2.116719117 |
| Cannbio_045448 | TMV resistance protein N-like protein (Fragment) | −7.137688019 |
| Cannbio_023213 | TMV resistance protein N-like protein (Fragment) | −7.757840978 |
| Cannbio_048101 | TMV resistance protein N-like protein (Fragment) | −7.811801695 |
| Cannbio_063072 | NB-ARC domain, LRR domain containing protein | −3.493241035 |
| Cannbio_064155 | NB-ARC domain, LRR domain containing protein | −5.405656385 |
| Cannbio_015516 | NB-ARC domain, LRR domain containing protein | −6.219562615 |
| Cannbio_039722 | Glucosyltransferase KGT15 (Fragment) | 2.64636655 |
| Cannbio_002736 | Truncated THCA synthase | 4.244311871 |
| Cannbio_045388 | THCA synthase (Fragment) | 4.235345849 |
| Cannbio_000877 | Truncated THCA synthase | 2.768064041 |
| Cannbio_005228 | Truncated THCA synthase | 4.805821801 |
| Cannbio_016204 | Cannabidiolic acid synthase | −2.322901165 |
| Cannbio_016865 | Cannabidiolic acid synthase | 6.828903098 |
| Cannbio_016114 | Cannabidiolic acid synthase-like 1 | −6.762821674 |
| Cannbio_013699 | Cannabidiolic acid synthase-like 1 | −3.099754086 |
| Cannbio_045994 | Olivetolic acid cyclase | 2.115249853 |
| Cannbio_000163 | Olivetolic acid cyclase | 2.410487315 |
| Cannbio_029154 | Olivetolic acid cyclase | 2.530528231 |
| Cannbio_034765 | Olivetolic acid cyclase | 3.551995235 |
| Cannbio_044427 | Olivetolic acid cyclase | 5.136059262 |
| Cannbio_030174 | Polyketide synthase 3 | 2.972080948 |
| Cannbio_029830 | Polyketide synthase 1 | 2.203037821 |
| Cannbio_042885 | Polyketide synthase 1 | 2.784372102 |
| Cannbio_048260 | Polyketide synthase 1 | 3.027918095 |
| Cannbio_034051 | Polyketide synthase 1 | 3.23530301 |
| Cannbio_036104 | Polyketide synthase 2 | 2.06662933 |
| Cannbio_001757 | Polyketide synthase 2 | 2.444379545 |
| Cannbio_039766 | Polyketide synthase 2 | 2.642472898 |
| Cannbio_037193 | Polyketide synthase 2 | 2.893451021 |
| Cannbio_060030 | Polyketide synthase 4 | −5.93270212 |
| Cannbio_001628 | Polyketide synthase 4 | 2.237284083 |
| Cannbio_039360 | Polyketide synthase 4 | 2.257952603 |
| Cannbio_009189 | Polyketide synthase 4 | 2.346004775 |
| Cannbio_039981 | U Polyketide synthase 4 | 2.579174682 |
| Cannbio_041647 | Polyketide synthase 4 | 2.802654648 |
| Cannbio_039530 | Polyketide synthase 4 | 2.906259499 |
| Cannbio_000101 | Polyketide synthase 4 | 3.114261613 |
| Cannbio_043103 | Polyketide synthase 5 | 2.913208238 |
| Cannbio_032283 | Polyketide synthase 5 | 2.976447257 |
| Cannbio_043358 | Polyketide synthase 5 | 3.171242303 |
| Cannbio_018356 | Polyketide synthase 5 | 3.365991945 |
| Cannbio_024998 | Chaicone synthase | 3.05058852 |
| Cannbio_015624 | Chaicone synthase | 4.196426416 |
| Cannbio_033975 | 2-acylphloroglucinol 4-prenyltransferase, chloroplastic | 3.34144641 |
| Cannbio_018250 | GPPS small subunit (Fragment) | 2.318372436 |
| Cannbio_056731 | Deoxyxylulose-5-phosphate synthase | −4.280270028 |
| Cannbio_023496 | 4-hydroxy-3-methylbut-2-en-l-yl diphosphate synthase, | 4.127229589 |
| | bacterial-type | |
| Cannbio_015609 | HDS (Fragment) | 2.087474823 |
| Cannbio_030486 | HDS (Fragment) | 2.541811067 |
| Cannbio_045663 | HDS (Fragment) | 2.565754256 |
| Cannbio_036932 | HDR (Fragment) | 2.080669572 |
| Cannbio_041807 | HDR (Fragment) | 2.111209448 |
| Cannbio_041763 | HDR (Fragment) | 2.152275693 |
| Cannbio_036741 | HDR (Fragment) | 2.18416714 |
| Cannbio_036684 | HDR (Fragment) | 2.198968518 |
| Cannbio_040192 | HDR (Fragment) | 2.298176471 |
| Cannbio_001482 | HDR (Fragment) | 2.316871683 |
| Cannbio_001860 | HDR (Fragment) | 2.449666658 |
| Cannbio_037729 | HDR (Fragment) | 2.489956785 |
| Cannbio_001692 | HDR (Fragment) | 2.596264591 |
| Cannbio_001663 | HDR (Fragment) | 2.61141185 |
| Cannbio_038822 | HDR (Fragment) | 2.70330901 |
| Cannbio_006736 | HDR (Fragment) | 2.774144793 |
| Cannbio_001749 | HDR (Fragment) | 2.880449204 |
| Cannbio_002273 | HDR (Fragment) | 2.95692054 |
| Cannbio_001569 | HDR (Fragment) | 3.015474833 |
| Cannbio_038936 | HDR (Fragment) | 3.032395383 |
| Cannbio_002181 | HDR (Fragment) | 3.060791817 |
| Cannbio_038048 | HDR (Fragment) | 3.185941647 |
| Cannbio_036416 | HDR (Fragment) | 3.324570455 |
| Cannbio_000796 | HDR (Fragment) | 4.000390123 |
| Cannbio_037034 | HDR (Fragment) | 4.082978261 |
| Cannbio_001191 | HDR (Fragment) | 4.201505332 |
| Cannbio_035170 | HDR (Fragment) | 4.415009655 |
| Cannbio_035649 | HDR (Fragment) | 4.660926305 |
| Cannbio_060627 | HDR (Fragment) | 5.863406404 |
| Cannbio_039298 | HDR (Fragment) | 6.327429125 |
| Cannbio_001253 | 4-hydroxy-3-methylbut-2-enyl diphosphate reductase | 2.51378082 |
| Cannbio_001301 | 4-hydroxy-3-methylbut-2-enyl diphosphate reductase | 2.6174184 |
| Cannbio_061752 | 4-hydroxy-3-methylbut-2-enyl diphosphate reductase | 2.734753321 |
| Cannbio_037930 | 4-hydroxy-3-methylbut-2-enyl diphosphate reductase | 2.947588473 |
| Cannbio_062278 | 2-C-methyl-D-erythritol 2,4-cyclodiphosphate synthase | −3.460466471 |
| Cannbio_056951 | Fatty acid desaturase | 4.911167062 |
| Cannbio_026331 | FAD2 (Fragment) | 6.190064397 |
| Cannbio_002293 | FA_desaturase domain-containing protein/DUF3474 | −4.886387402 |
| | domain-containing protein | |
| Cannbio_001709 | Omega-6 fatty acid desaturase, endoplasmic reticulum | 7.032989849 |
| | isozyme 1-like | |
| Cannbio_003201 | Omega-6 fatty acid desaturase, endoplasmic reticulum | 3.023461637 |
| | isozyme 2 | |
| Cannbio_016317 | Terpene synthase | 2.807645607 |
| Cannbio_006443 | Terpene synthase | 3.311759866 |
| Cannbio_026189 | Terpene synthase | 3.414935376 |
| Cannbio_021413 | Terpene synthase | 3.712911527 |
| Cannbio_041126 | Terpene synthase | 2.055641301 |
| Cannbio_006127 | Terpene synthase | 3.296399073 |
| Cannbio_036336 | Terpene synthase | 3.410803951 |
| Cannbio_048198 | Terpene synthase | 2.042331097 |
| Cannbio_039793 | Terpene synthase | 2.199117028 |
| Cannbio_019641 | Terpene synthase | 3.220518811 |
| Cannbio_035365 | Terpene synthase | 3.268774607 |
| Cannbio_026571 | Terpene synthase | 3.556555046 |
| Cannbio_021373 | Terpene synthase | 4.164225268 |
| Cannbio_006875 | Terpene synthase | 4.985723573 |
| Cannbio_032731 | Terpene synthase | 5.214156405 |
| Cannbio_043531 | Terpene synthase | 5.277265609 |
| Cannbio_014047 | Terpene synthase | 5.381277932 |
| Cannbio_029651 | Terpene synthase | 5.694324717 |
| Cannbio_031172 | Terpene synthase | 5.720143156 |
| Cannbio_020910 | Terpene synthase | 5.768444649 |
| Cannbio_001051 | Terpene synthase | 6.422695862 |
| Cannbio_012855 | Terpene synthase | 3.571079283 |
| Cannbio_000746 | Terpene synthase | 4.236177459 |
| Cannbio_037841 | Terpene synthase | 4.237439335 |
| Cannbio_024851 | Terpene synthase | 4.416800193 |
| Cannbio_026852 | Terpene synthase | 5.967129273 |
| Cannbio_037103 | Terpene synthase | 6.641335768 |
| Cannbio_004731 | Terpene synthase | −7.142682378 |
| Cannbio_006565 | Terpene synthase | 2.178685631 |
| Cannbio_013743 | Terpene synthase | 2.201374551 |
| Cannbio_043909 | Terpene synthase | 3.294183954 |
| Cannbio_048110 | Terpene synthase | 3.629620139 |
| Cannbio_039060 | Terpene synthase | 3.904630491 |
| Cannbio_001375 | Terpene synthase | 4.002843914 |
| Cannbio_009119 | Terpene synthase | 4.439477489 |
| Cannbio_048188 | Terpene synthase | 6.813376172 |
| Cannbio_023581 | Terpene synthase | 2.035646177 |
| Cannbio_017410 | Terpene synthase | 2.045473581 |
| Cannbio_006417 | Terpene synthase | 2.144780258 |
| Cannbio_030713 | Terpene synthase | 2.654768329 |
| Cannbio_006553 | Terpene synthase | 2.748358471 |
| Cannbio_043040 | Terpene synthase | 5.703266846 |
| Cannbio_033511 | Terpene synthase | 4.195522506 |
| Cannbio_045040 | Terpene synthase | 4.676096452 |
| Cannbio_022866 | Terpene synthase | 4.737206419 |
| Cannbio_029777 | Terpene synthase | 4.876586342 |
| Cannbio_019267 | Terpene synthase | 5.150276266 |
| Cannbio_016508 | Terpene cyclase/mutase family member | −2.507211401 |
| Cannbio_029679 | Terpene cyclase/mutase family member | 3.529230197 |
| Cannbio_032888 | (−)-limonene synthase, chloroplastic | 4.12252482 |
| Cannbio_039860 | (−)-limonene synthase, chloroplastic | 4.182273223 |
| Cannbio_030130 | (−)-limonene synthase, chloroplastic | 4.428959546 |
| Cannbio_033300 | (−)-limonene synthase, chloroplastic | 5.224047488 |
| Cannbio_040185 | (−)-limonene synthase, chloroplastic | 5.343118637 |
| Cannbio_032834 | (−)-limonene synthase, chloroplastic | 5.370604027 |
| Cannbio_012008 | (−)-limonene synthase, chloroplastic | 5.421705163 |
| Cannbio_006811 | (−)-limonene synthase, chloroplastic | 5.437689179 |
| Cannbio_032659 | (−)-limonene synthase, chloroplastic | 5.453220133 |
| Cannbio_041779 | (−)-limonene synthase, chloroplastic | 5.547990502 |
| Cannbio_022649 | (−)-limonene synthase, chloroplastic | 5.587056887 |
| Cannbio_034925 | (+)-alpha-pinene synthase, chloroplastic | 3.77186841 |
| Cannbio_025638 | (+)-alpha-pinene synthase, chloroplastic | 5.415973129 |
| Cannbio_016048 | (+)-alpha-pinene synthase, chloroplastic | 6.468651338 |
| Cannbio_041037 | 3,5,7-trioxododecanoyl-CoA synthase | 2.110187551 |
| Cannbio_048356 | 3,5,7-trioxododecanoyl-CoA synthase | 2.1823627 |
| Cannbio_044836 | 3,5,7-trioxododecanoyl-CoA synthase | 2.324459659 |
| Cannbio_001409 | 3,5,7-trioxododecanoyl-CoA synthase | 2.338889023 |
| Cannbio_004873 | 3,5,7-trioxododecanoyl-CoA synthase | 2.372511535 |
| Cannbio_008449 | 3,5,7-trioxododecanoyl-CoA synthase | 2.63436839 |
| Cannbio_019717 | 3,5,7-trioxododecanoyl-CoA synthase | 3.044871644 |
| Cannbio_049974 | lupeol synthase isoform X2 | −5.310715597 |
| Cannbio_014174 | Secologanin synthase | 5.706266698 |
| Cannbio_021743 | Secologanin synthase | 6.532459646 |
| Cannbio_003185 | Secologanin synthase | 7.586772477 |
| Cannbio_025700 | Secologanin synthase | 7.684694611 |
| Cannbio_022533 | Secologanin synthase | 9.33455015 |
| Cannbio_023316 | Vinorine synthase | −4.584314909 |
| Cannbio_013204 | MADS-box transcription factor | −2.23847317 |
| Cannbio_059903 | MADS-box transcription factor | −2.282089091 |
| Cannbio_013942 | MADS-box transcription factor | 2.169657448 |
| Cannbio_006389 | MADS-box transcription factor | 3.290594582 |
| Cannbio_049455 | MADS-box transcription factor | 3.153489021 |
| Cannbio_050725 | MADS-box transcription factor | 4.371534215 |
| Cannbio_010630 | MADS-box transcription factor | 3.955810951 |
| Cannbio_001292 | MADS-box transcription factor | 3.957888555 |
| Cannbio_052623 | MADS-box transcription factor | 4.079963356 |
| Cannbio_038827 | MADS-box transcription factor | 4.165160941 |
| Cannbio_002936 | MADS-box transcription factor | 6.041234714 |
| Cannbio_030355 | MADS-box transcription factor | −2.407116685 |
| Cannbio_029230 | MADS-box transcription factor | 4.851031062 |
| Cannbio_050616 | MADS-box transcription factor | −2.566231064 |
| Cannbio_009872 | MADS-box transcription factor | −6.237151838 |
| Cannbio_057623 | MADS-box transcription factor | −2.160519812 |
| Cannbio_053021 | MADS-box transcription factor | 2.866520911 |
| Cannbio_048850 | MADS-box transcription factor | 3.679732926 |
| Cannbio_062967 | MADS-box transcription factor | −2.905221231 |
| Cannbio_053844 | MADS-box transcription factor | −3.623331194 |
| Cannbio_009169 | MADS-box transcription factor | −3.420492431 |
| Cannbio_062969 | MADS-box transcription factor | −3.376921409 |
| Cannbio_062122 | Agamous-like MADS-box protein AGL104 | −3.34720931 |
| Cannbio_017957 | Agamous-like mads-box protein agl8-like (Fragment) | 2.986229352 |
| Cannbio_020814 | MADS-box transcription factor 17 isoform X2 | 3.07999278 |

Differentially Expressed Genes Associated with Female Cannabis Plant Development

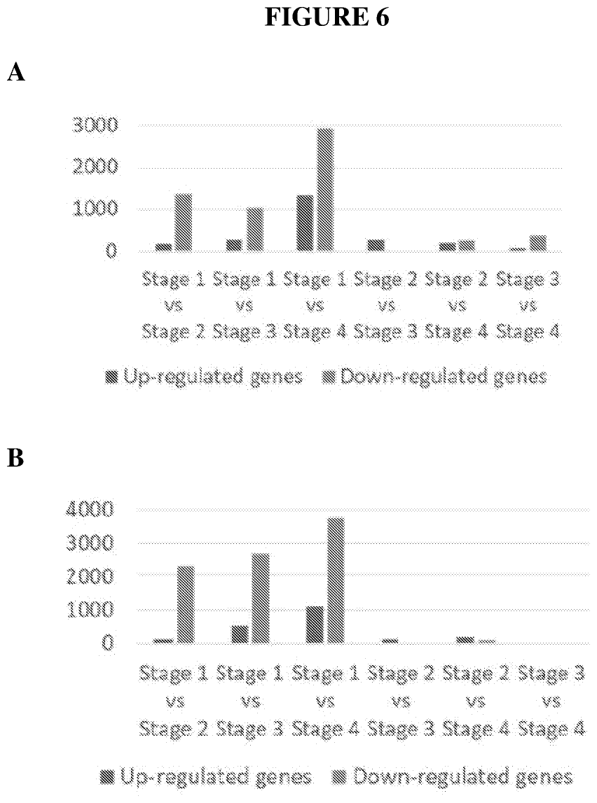

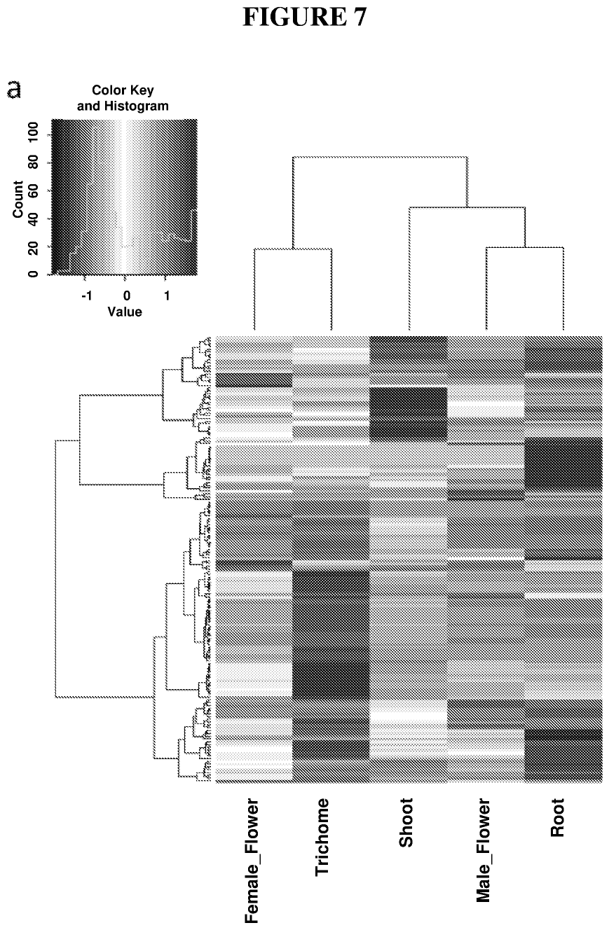

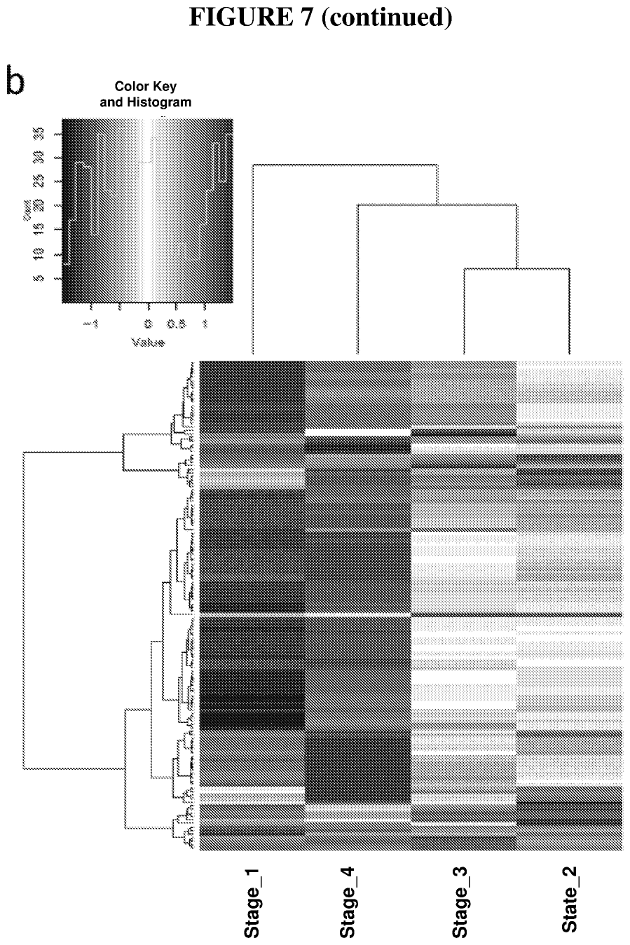

The number of genes that were identified to be differentially expressed across various developmental stages in female flowers and trichome tissues were also analysed and are represented in FIGS. 6A and B. It was found that developmental Stage 1 had the most divergent dataset when compared to all other stages in terms of gene expression. A notable increase in the number of up-regulated genes was observed at Stage 4 when compared to Stage 3, Stage 2 and Stage 1 in both the female flowers and trichomes. For instance, Stage 1 (immature floral bud) when compared to Stage 4 (mature floral bud) had 4,274 (31.2% up-regulated and 68.8% down-regulated genes) and 4,854 (22.6% up-regulated and 77.4% down-regulated genes) differentially expressed genes in female flowers and trichomes respectively. The genes that were found to be frequently up-regulated in Stage 1 when compared to Stage 4 in female flowers and trichomes had similar gene annotations; for example, sieve element occlusion, lipase, cytochrome P450 and fatty acid hydroxylase. In female flowers, the gene expression was observed to change the least in Stage 2 when compared to Stage 3 (296 genes), followed by either Stages 2 and 3 as compared to Stage 4. Whereas in trichomes, the least expression change was found in Stage 3 when compared to Stage 4 (37 genes), followed by Stage 2 as compared to Stages 3 and 4. A summary of differentially expressed genes identified based on comparisons made across the female reproductive developmental stages with their UniRef100 annotations and log₂Fold Change are detailed in Tables 12-14.

TABLE 12
Differentially expressed genes across developmental Stage 2 in trichomes
and flowers with log2FoldChange and UniRef100 annotation.

| | | | Log2FoldChange | |
|---|---|---|---|---|
| | | | (Stage 1 v Stage 2) | |
| UniRef100 Annotation | Name | Taxonomy | Trichome | Flower |
| UniRef100_A0A022S0R8 | Beta-galacto sidase | Erythranthe guttata | −4.3622 | −3.31517 |
| UniRef100_A0A061FPX4 | Serine protease inhibitor | Theobroma cacao | −4.16011 | −3.4908 |
| UniRef100_A0A078JZ04 | BnaCnng70250D protein | Brassica napus | −3.38775 | −2.76967 |
| | (Fragment) | | | |
| UniRef100_A0A088MER7 | Delta 15 desaturase | Cannabis sativa | −2.89929 | −2.20703 |
| UniRef100_A0A088MFF4 | Delta 12 desaturase | Cannabis sativa | −4.02657 | −2.12514 |
| UniRef100_A0A0A9TKE0 | Gdh1 | Arundo donax | −3.62545 | −2.82488 |
| UniRef100_A0A0B0PET0 | Beta-D-xylosidase 1-like protein | Gossypium arboreum | −5.13941 | −3.75324 |
| UniRef100_A0A0N9DV50 | Polyphenol oxidase | Morus alba var. | −4.04457 | −4.22869 |
| | | multicaulis | | |
| UniRef100_A0A0P0WD08 | Os04g0543900 protein | Oryza sativa subsp. | −4.24105 | −2.83673 |
| | | japonica | | |
| UniRef100_A0A1Q3CSP5 | Cu_bind_like domain-containing | Cephalotus follicularis | −3.3454 | −3.36634 |
| | protein | | | |
| UniRef100_A0A1R3KRM8 | Oxoglutarate/iron-dependent | Corchorus olitorius | −3.48822 | −2.95196 |
| | dioxygenase | | | |
| UniRef100_A0A1S3AWG6 | Beta-amylase | Cucumis melo | −2.89361 | −3.18539 |
| UniRef100_A0A1U8E1W5 | Beta-galactosidase | Capsicum annuum | −4.4946 | −3.66929 |
| UniRef100_A0A1U8EAR7 | Beta-galactosidase | Capsicum annuum | −4.47218 | −4.21076 |
| UniRef100_A0A1V0QSH9 | HDR (Fragment) | Cannabis sativa | −2.675 | −2.18403 |
| UniRef100_A0A200PTX7 | Aldo/keto reductase | Macleaya cordata | −5.2121 | −5.75935 |
| UniRef100_A0A218W4J0 | Laccase | Punica granatum | −4.69042 | −4.13107 |
| UniRef100_A0A251MVM4 | Laccase | Primus persica | −3.30323 | −3.23955 |
| UniRef100_A0A251RLN7 | Putative arabinose kinase | Helianthus annuus | −4.97466 | −3.54534 |
| UniRef100_A0A2I0B6H1 | Retrovirus-related Pol polyprotein | Apostasia shenzhenica | −2.1207 | −2.61315 |
| | from transposon TNT 1-94 | | | |
| UniRef100_A0A2I0VWY1 | Putative mitochondrial protein | Dendrobium | −2.31158 | −2.57711 |
| | | catenatum | | |
| UniRef100_A0A2I0WB13 | Retrovirus-related Pol polyprotein | Dendrobium | −3.41842 | −2.84327 |
| | from transposon TNT 1-94 | catenatum | | |
| UniRef100_A0A2I4EN88 | protein NUCLEAR FUSION | Juglans regia | −4.34454 | −3.42227 |
| | DEFECTIVE 4-like | | | |
| UniRef100_A0A2I4HEZ5 | stellacyanin-like | Juglans regia | −3.49387 | −3.14343 |
| UniRef100_A0A2K2A2D7 | Beta-galactosidase | Populus trichocarpa | −4.48252 | −4.31542 |
| UniRef100_A0A2K2BW72 | Amine oxidase | Populus trichocarpa | −2.44289 | −2.01836 |
| UniRef100_A0A2K3LFB9 | Asparagine synthetase (Fragment) | Trifolium pratense | −4.20294 | −2.5741 |
| UniRef100_A0A2K3NBQ9 | Retrovirus-related Pol polyprotein | Trifolium pratense | −2.78654 | −2.16164 |
| | from transposon TNT 1-94 | | | |
| UniRef100_A0A2K3NEN7 | Copia-like polyprotein (Fragment) | Trifolium pratense | −2.23516 | −3.65317 |
| UniRef100_A0A2N9II53 | Pectinesterase | Fagus sylvatica | −4.54124 | −2.40666 |
| UniRef100_A0A2P4HR67 | Gibberellin 3-beta-dioxygenase 1 | Quercus suber | −6.64605 | −5.86723 |
| UniRef100_A0A2P5A4I7 | Transferase | Trema orientalis | −2.27592 | −2.92483 |
| UniRef100_A0A2P5AFS5 | Protein IDA-LIKE | Parasponia andersonii | −2.16756 | −2.19947 |
| UniRef100_A0A2P5AG05 | Cytochrome P450, E-class, group I | Trema orientalis | −4.11597 | −2.21371 |
| UniRef100_A0A2P5AHF4 | ABC-2 type transporter | Cannabaceae | −2.50168 | −2.67877 |
| UniRef100_A0A2P5AHI9 | Coatomer beta subunit | Parasponia andersonii | −3.89007 | −2.18058 |
| UniRef100_A0A2P5AJS6 | Lipase | Parasponia andersonii | −3.03529 | −3.13738 |
| UniRef100_A0A2P5AJT1 | Cytochrome P450, E-class, group I | Trema orientalis | −3.38978 | −2.0219 |
| UniRef100_A0A2P5AKR0 | Cytochrome P450, E-class, group I | Trema orientalis | −4.41055 | −2.16175 |
| UniRef100_A0A2P5ALD0 | Wall-associated receptor kinase | Parasponia andersonii | −2.08485 | −2.05823 |
| UniRef100_A0A2P5ALM2 | PQ-loop repeat | Parasponia andersonii | −3.18961 | −3.3625 |
| UniRef100_A0A2P5ALY5 | Allene oxide synthase | Trema orientalis | −2.30123 | −2.86528 |
| UniRef100_A0A2P5AP23 | LURP1-related protein domain | Trema orientalis | −5.1574 | −4.52901 |
| | containing protein | | | |
| UniRef100_A0A2P5APY4 | Long-chain-alcohol oxidase | Parasponia andersonii | −4.68872 | −4.27056 |
| UniRef100_A0A2P5APY6 | Long-chain-alcohol oxidase | Parasponia andersonii | −4.63354 | −4.39774 |
| UniRef100_A0A2P5APZ2 | Long-chain-alcohol oxidase | Parasponia andersonii | −4.53984 | −3.74645 |
| UniRef100_A0A2P5AQ01 | Flavin-containing monooxygenase | Trema orientalis | −3.00633 | −3.45523 |
| UniRef100_A0A2P5AQ09 | Long-chain-alcohol oxidase | Parasponia andersonii | −5.2589 | −3.77559 |
| UniRef100_A0A2P5AQ43 | TFIIH C1-like domain containing | Trema orientalis | −2.43421 | −3.31339 |
| | protein | | | |
| UniRef100_A0A2P5AQK5 | Xanthine dehydrogenase C subunit | Parasponia andersonii | −6.1842 | −3.20413 |
| UniRef100_A0A2P5ARH7 | Xanthine dehydrogenase C subunit | Parasponia andersonii | −2.17594 | −2.00627 |
| UniRef100_A0A2P5ARN0 | Hopanoid-associated sugar | Trema orientalis | −3.74504 | −2.00142 |
| | epimerase | | | |
| UniRef100_A0A2P5ARP8 | Tetratricopeptide-like helical | Parasponia andersonii | −3.03427 | −2.34621 |
| | domain containing protein | | | |
| UniRef100_A0A2P5ATD1 | Fungal lipase-like domain | Parasponia andersonii | −2.1707 | −2.42027 |
| | containing protein | | | |
| UniRef100_A0A2P5ATT8 | Gibberellin-3 oxidase | Parasponia andersonii | −5.33552 | −4.56421 |
| UniRef100_A0A2P5AUT8CX2CX4HX4C | Zinc knuckle | Trema orientalis | −3.15075 | −2.18415 |
| UniRef100_A0A2P5AUW0 | ACT domain containing protein | Parasponia andersoni | −2.27135 | −2.19932 |
| UniRef100_A0A2P5AWP9 | Cytochrome P450, E-class, group I | Trema orientalis | −3.05366 | −3.85903 |
| UniRef100_A0A2P5AWV8 | Peroxidase | Parasponia andersoni | −4.67835 | −2.75994 |
| UniRef100_A0A2P5AZ53 | Transferase | Parasponia andersonii | −2.78598 | −2.16688 |
| UniRef100_A0A2P5B3S9 | Cytochrome P450, E-class, group I | Parasponia andersonii | −2.37237 | −2.41798 |
| | (Fragment) | | | |
| UniRef100_A0A2P5B568 | Hexosyltransferase | Trema orientalis | −5.06422 | −5.02231 |
| UniRef100_A0A2P5B715 | Developmental regulator, | Parasponia andersonii | −3.39566 | −2.52404 |
| | ULTRAPETALA | | | |
| UniRef100_A0A2P5B8L7 | Lipase | Parasponia andersonii | −4.45778 | −2.75361 |
| UniRef100_A0A2P5B9I0 | Oxoglutarate/iron-dependent | Trema orientalis | −3.66762 | −3.60673 |
| | dioxygenase | | | |
| UniRef100_A0A2P5B9T6 | Cytochrome P | Trema orientalis | −2.41807 | −2.18919 |
| UniRef100_A0A2P5BAR9 | Laccase | Parasponia andersonii | −3.41275 | −2.93407 |
| UniRef100_A0A2P5BED8 | Zf-FLZ domain containing protein | Parasponia andersonii | −3.59377 | −3.27411 |
| UniRef100_A0A2P5BEV1 | Pectinesterase | Parasponia andersonii | −4.34984 | −3.85283 |
| UniRef100_A0A2P5BFW6 | Peroxidase | Trema orientalis | −2.69562 | −2.12894 |
| UniRef100_A0A2P5BG65 | Caleosin-related | Trema orientalis | −3.14046 | −2.95785 |
| UniRef100_A0A2P5BJ37 | Fatty acid desaturase | Parasponia andersonii | −2.25465 | −2.6655 |
| UniRef100_A0A2P5BJ92 | Peptidase T2, asparaginase | Trema orientalis | −5.09643 | −2.73535 |
| UniRef100_A0A2P5BJZ7 | Oxoglutarate/iron-dependent | Trema orientalis | −5.26815 | −4.37088 |
| | dioxygenase (Fragment) | | | |
| UniRef100_A0A2P5BKA3 | Bifunctional inhibitor/plant lipid | Parasponia andersonii | −2.07834 | −2.58872 |
| | transfer protein/seed storage | | | |
| | helical domain containing protein | | | |
| UniRef100_A0A2P5BKM3 | Equilibrative nucleoside | Parasponia andersonii | −3.19491 | −2.50141 |
| | transporter | | | |
| UniRef100_A0A2P5BLP6 | NB-ARC domain containing | Trema orientalis | −3.7084 | −3.38228 |
| | protein | | | |
| UniRef100_A0A2P5BNZ9 | Beta-galactosidase | Parasponia andersonii | −5.80462 | −2.94748 |
| UniRef100_A0A2P5BP34 r | Sugar/inositol transporte | Parasponia andersonii | −4.11243 | −4.93713 |
| UniRef100_A0A2P5BPI7 | 2,3-dihydroxybenzoate-AMP | Parasponia andersonii | −3.6275 | −4.90846 |
| | ligase | | | |
| UniRef100_A0A2P5BPK0 | Polyphenol oxidase | Parasponia andersonii | −4.29162 | −3.27903 |
| UniRef100_A0A2P5BSG5 | Glycoside hydrolase | Trema orientalis | −4.56081 | −2.99522 |
| UniRef100_A0A2P5BTK0 | Stigma-specific protein | Parasponia andersonii | −3.00102 | −2.73892 |
| UniRef100_A0A2P5BUT0 | Pectinesterase | Trema orientalis | −2.11774 | −2.63597 |
| UniRef100_A0A2P5BV01 | S-receptor-like serine/threonine- | Parasponia andersonii | −4.04523 | −3.4824 |
| | protein kinase | | | |
| UniRef100_A0A2P5BV07 | Beta-galactosidase | Trema orientalis | −3.11834 | −2.8457 |
| UniRef100_A0A2P5BWT4 | Proteinase inhibitor | Parasponia andersonii | −4.6546 | −2.7149 |
| UniRef100_A0A2P5BXS8 | Peroxidase | Parasponia andersonii | −3.44934 | −2.82777 |
| UniRef100_A0A2P5BYA8 | Cytochrome P | Parasponia andersonii | −3.62112 | −2.47521 |
| UniRef100_A0A2P5BYH9 | Oxysterol-binding protein | Parasponia andersonii | −2.58635 | −2.30437 |
| UniRef100_A0A2P5BZ09 | Lipase | Parasponia andersonii | −2.09888 | −3.29298 |
| UniRef100_A0A2P5C078 | Ulpl protease family, C-terminal | Trema orientalis | −5.08281 | −2.61176 |
| | catalytic domain containing | | | |
| | protein | | | |
| UniRef100_A0A2P5C2A9 | Cysteine-rich secretory protein, | Parasponia andersonii | −5.84589 | −4.49684 |
| | allergen V5/Tpx-1-related | | | |
| UniRef100_A0A2P5C2F3 | Lipid transfer protein/Par allergen | Parasponia andersonii | −2.26294 | −3.19495 |
| UniRef100_A0A2P5C2X8 | Cytochrome P450, E-class, group I | Parasponia andersonii | −2.63796 | −2.19035 |
| UniRef100_A0A2P5C4M1 | Beta-glucanase | Parasponia andersonii | −4.81104 | −2.89915 |
| UniRef100_A0A2P5C5Z2 | Purple acid phosphatase | Trema orientalis | −3.69578 | −2.20228 |
| UniRef100_A0A2P5C6L7 | Proton-dependent oligopeptide | Parasponia andersonii | −5.23674 | −4.15709 |
| | transporter | | | |
| UniRef100_A0A2P5C806 | Bidirectional sugar transporter | Parasponia andersonii | −3.27615 | −3.62662 |
| | SWEET | | | |
| UniRef100_A0A2P5C9G9 | Cellulose synthase | Parasponia andersonii | −4.34162 | −2.14332 |
| UniRef100_A0A2P5CCN3 | Phytocyanin domain containing | Parasponia andersonii | −3.88385 | −3.27938 |
| | protein | | | |
| UniRef100_A0A2P5CEF5 | Spastin | Parasponia andersonii | −4.87728 | −3.93926 |
| UniRef100_A0A2P5CFA7 | Major facilitator | Parasponia andersonii | −3.41209 | −2.72345 |
| UniRef100_A0A2P5CGK6 | Peroxidase | Trema orientalis | −2.82628 | −3.28041 |
| UniRef100_A0A2P5CHC7 | ABC transporter-like | Trema orientalis | −4.03867 | −3.51937 |
| UniRef100_A0A2P5CI93 | Xyloglucan | Trema orientalis | −4.55912 | −3.15207 |
| | endotransglucosylase/hydrolase | | | |
| UniRef100_A0A2P5CIB0 | Xyloglucan | Trema orientalis | −3.67931 | −3.12056 |
| | endotransglucosylase/hydrolase | | | |
| UniRef100_A0A2P5CJE4 | Oxoglutarate/iron-dependent | Trema orientalis | −4.74309 | −3.60442 |
| | dioxygenase | | | |
| UniRef100_A0A2P5CK43 | GRAS transcription factor | Parasponia andersonii | −3.67899 | −2.35504 |
| UniRef100_A0A2P5CKG8 | Amino acid transporter, | Trema orientalis | −4.89732 | −3.45277 |
| | transmembrane domain containing | | | |
| | protein | | | |
| UniRef100_A0A2P5CKT0 | Serine/threonine protein kinase | Trema orientalis | −3.3746 | −3.42474 |
| UniRef100_A0A2P5CLJ4 | Pectinesterase | Trema orientalis | −3.68954 | −2.20351 |
| UniRef100_A0A2P5CLL1 | Glycoside hydrolase | Parasponia andersonii | −4.58919 | −3.21323 |
| UniRef100_A0A2P5CNB4 | Lipase | Trema orientalis | −4.16836 | −2.34889 |
| UniRef100_A0A2P5CQX7 | 2,3-dihydroxybenzoate-AMP | Parasponia andersonii | −4.53256 | −2.88996 |
| | ligase | | | |
| UniRef100_A0A2P5CQZ3 | Pectinesterase | Parasponia andersonii | −3.73289 | −4.54384 |
| UniRef100_A0A2P5CTX8 | Beta-galactosidase | Parasponia andersonii | −5.18873 | −3.78196 |
| UniRef100_A0A2P5CWB3 | N-terminal acetyltransferase A, | Trema orientalis | −2.21342 | −2.21689 |
| | auxiliary subunit | | | |
| UniRef100_A0A2P5CX70 | Wall-associated receptor kinase | Trema orientalis | −2.19617 | −2.08117 |
| UniRef100_A0A2P5CX78 | Exostosin-like | Trema orientalis | −3.56416 | −2.72887 |
| UniRef100_A0A2P5CXK7 | Bifunctional inhibitor/plant lipid | Parasponia andersonii | −4.77495 | −3.28917 |
| | transfer protein/seed storage | | | |
| | helical domain containing protein | | | |
| UniRef100_A0A2P5CYN2 | Non-specific lipid-transfer protein | Trema orientalis | −4.97347 | −2.17392 |
| UniRef100_A0A2P5D1D4 | Sugar/inositol transporter | Parasponia andersonii | −4.0238 | −3.69843 |
| UniRef100_A0A2P5D1N7 | Isopenicillin N synthase | Trema orientalis | −2.25131 | −2.25341 |
| UniRef100_A0A2P5D2V7 | GH3-like hormone conjugating | Trema orientalis | −4.11174 | −2.80365 |
| | enzyme | | | |
| UniRef100_A0A2P5D3M2 | Vacuolar protein sorting- | Trema orientalis | −2.03491 | −2.12792 |
| | associated protein | | | |
| UniRef100_A0A2P5D400 | Metallothionein-like protein type | Parasponia andersonii | −4.35003 | −2.09191 |
| UniRef100_A0A2P5D5M9 | DNA-directed DNA polymerase | Parasponia andersonii | −3.65261 | −2.87778 |
| UniRef100_A0A2P5D5P4 | LRR domain containing protein | Parasponia andersonii | −3.51962 | −2.00998 |
| UniRef100_A0A2P5D713 | AP2/ERF transcription factor | Parasponia andersonii | −2.49738 | −2.90364 |
| UniRef100_A0A2P5D7Q8 | Exostosin-like | Trema orientalis | −2.61544 | −2.05494 |
| UniRef100_A0A2P5D8V4 | Lipoxygenase | Parasponia andersonii | −2.12329 | −3.57513 |
| UniRef100_A0A2P5D8W1 | Glycoside hydrolase | Parasponia andersonii | −4.45289 | −2.95757 |
| UniRef100_A0A2P5D923 | Proteinase inhibitor 13, Kunitz | Parasponia andersonii | −2.84357 | −2.29253 |
| | legume | | | |
| UniRef100_A0A2P5D946 | Proteinase inhibitor 13, Kunitz | Parasponia andersonii | −2.81467 | −3.89894 |
| | legume | | | |
| UniRef100_A0A2P5DA80 | Leucine-rich repeat domain | Trema orientalis | −4.22832 | −2.04236 |
| | containing protein | | | |
| UniRef100_A0A2P5DAM5 | Cytochrome P450, E-class, group I | Trema orientalis | −3.6911 | −2.72598 |
| UniRef100_A0A2P5DB33x | Cytochrome P450, E-class, group I | Trema orientalis | −3.41806 | −3.16866 |
| UniRef100_A0A2P5DBV5 | 1,4-alpha-glucan-branching | Parasponia andersonii | −3.62821 | −2.00061 |
| | enzyme | | | |
| UniRef100_A0A2P5DCT9x | 1,4-alpha-glucan-branching | Parasponia andersonii | −3.06011 | −2.84434 |
| | enzyme | | | |
| UniRef100_A0A2P5DGE2 | Transmembrane protein | Trema orientalis | −4.99637 | −2.90746 |
| UniRef100_A0A2P5DGM4 | Zinc finger, RING-CH-type | Trema orientalis | −3.54814 | −2.7298 |
| UniRef100_A0A2P5DKL8 x | Zinc finger, RING-CH-type | Trema orientalis | −4.38829 | −2.95921 |
| UniRef100_A0A2P5DL95 | WRKY domain containing protein | Trema orientalis | −2.29306 | −2.20105 |
| UniRef100_A0A2P5DQU9 x | WRKY domain containing protein | Trema orientalis | −2.16977 | −2.5062 |
| UniRef100_A0A2P5DS59 x | WRKY domain containing protein | Trema orientalis | −3.80059 | −3.03104 |
| UniRef100_A0A2P5DTC9 | Aspartate aminotransferase | Parasponia andersonii | −3.87032 | −2.74963 |
| UniRef100_A0A2P5DU09 x | Aspartate aminotransferase | Parasponia andersonii | −3.01168 | −2.4372 |
| UniRef100_A0A2P5DVB1 | Bidirectional sugar transporter | Parasponia andersonii | −3.97124 | −2.7501 |
| | SWEET | | | |
| UniRef100_A0A2P5DVB6 | Acyl- [acy 1-carrier-protein] | Parasponia andersonii | −2.67147 | −2.53449 |
| | hydrolase (Fragment) | | | |
| UniRef100_A0A2P5DVC2 | Transferase | Parasponia andersonii | −2.34872 | −2.8801 |
| UniRef100_A0A2P5DWC8 | Cytochrome P450, E-class, group I | Parasponia andersonii | −2.82099 | −2.28574 |
| UniRef100_A0A2P5DWQ6 | Glycoside hydrolase | Parasponia andersonii | −3.57345 | −2.39978 |
| UniRef100_A0A2P5DWT3 | Endoglucanase | Parasponia andersonii | −3.83236 | −4.05082 |
| UniRef100_A0A2P5DYA6 | Ubiquitin-fold modifier- | Trema orientalis | −2.14977 | −2.97221 |
| | conjugating enzyme | | | |
| UniRef100_A0A2P5DYW8 | Cytochrome P450, E-class, group I | Trema orientalis | −3.50353 | −3.48459 |
| UniRef100_A0A2P5DZ03 | Glycoside hydrolase | Parasponia andersonii | −3.25899 | −2.06236 |
| UniRef100_A0A2P5E0I2 e | non-specific serine/threonine | Parasponia andersonii | −2.78556 | −2.71748 |
| | protein kinas | | | |
| UniRef100_A0A2P5E1C4 | Long-chain-alcohol oxidase | Trema orientalis | −5.52216 | −3.15011 |
| UniRef100_A0A2P5E1G4 | Long-chain-alcohol oxidase | Trema orientalis | −5.20808 | −2.89499 |
| UniRef100_A0A2P5E3B4 | Bifunctional inhibitor/plant lipid | Parasponia andersonii | −2.76758 | −4.26577 |
| | transfer protein/seed storage | | | |
| | helical domain containing protein | | | |
| UniRef100_A0A2P5E3K4 | Sugar/inositol transporter | Parasponia andersonii | −3.25922 | −2.16896 |
| UniRef100_A0A2P5E402 | CASP-like protein | Parasponia andersonii | −4.30892 | −2.88198 |
| UniRef100_A0A2P5E629 | Beta-galactosidase | Trema orientalis | −5.93424 | −2.73353 |
| UniRef100_A0A2P5E6N2 | Glycoside hydrolase | Trema orientalis | −3.89619 | −2.90725 |
| UniRef100_A0A2P5E6P5 | Small auxin-up RNA | Trema orientalis | −3.42444 | −2.16229 |
| UniRef100_A0A2P5E7T8 | 43kDa postsynaptic protein | Trema orientalis | −2.13191 | −2.16683 |
| UniRef100_A0A2P5E838 | Oxoglutarate/iron-dependent | Trema orientalis | −4.54121 | −3.60521 |
| | dioxygenase | | | |
| UniRef100_A0A2P5E9F5 | Peroxidase | Trema orientalis | −3.77542 | −2.52546 |
| UniRef100_A0A2P5E9U8 | SAM dependent carboxyl | Trema orientalis | −3.69387 | −2.43308 |
| | methyltransferase | | | |
| UniRef100_A0A2P5EAQ9 | GH3-like hormone conjugating | Trema orientalis | −5.31959 | −4.23537 |
| | enzyme | | | |
| UniRef100_A0A2P5EB08 | Rhodanese-like domain containing | Trema orientalis | −2.67452 | −2.31332 |
| | protein | | | |
| UniRef100_A0A2P5EB64 | TRAM/LAG1/CLN8 domain | Trema orientalis | −3.88515 | −2.0442 |
| | containing protein | | | |
| UniRef100_A0A2P5ECS7 | Proteinase inhibitor | Trema orientalis | −4.3063 | −3.35033 |
| UniRef100_A0A2P5ECX7 | Small auxin-up RNA | Trema orientalis | −2.1746 | −2.36096 |
| UniRef100_A0A2P5EDE0 | Cellulose synthase | Trema orientalis | −2.93453 | −2.75233 |
| UniRef100_A0A2P5EE60 | DREPP family | Trema orientalis | −4.10411 | −2.58989 |
| UniRef100_A0A2P5EGL6 | Non-specific serine/threonine | Trema orientalis | −3.20525 | −2.51239 |
| | protein kinase | | | |
| UniRef100_A0A2P5EGN6 | Peptidase T2, asparaginase | Trema orientalis | −6.09271 | −3.38855 |
| UniRef100_A0A2P5EHM4 | V-type proton ATPase subunit G | Trema orientalis | −2.53036 | −2.69119 |
| UniRef100_A0A2P5EI74 | Cytochrome P450, E-class, group I | Trema orientalis | −3.41994 | −3.86429 |
| UniRef100_A0A2P5EI95 | Cysteine protease | Trema orientalis | −5.35056 | −3.87903 |
| UniRef100_A0A2P5EIG2 | Peroxidase | Trema orientalis | −5.6368 | −2.94837 |
| UniRef100_A0A2P5EIM8 | Beta-galactosidase | Trema orientalis | −5.78526 | −4.63672 |
| UniRef100_A0A2P5EJ68 | Carotenoid cleavage dioxygenase | Trema orientalis | −2.60339 | −4.06404 |
| UniRef100_A0A2P5EJV0 | Equilibrative nucleoside | Trema orientalis | −2.85869 | −2.05911 |
| | transporter | | | |
| UniRef100_A0A2P5EK07 | Laccase | Trema orientalis | −4.10183 | −3.74568 |
| UniRef100_A0A2P5EN03 | Serine/threonine protein kinase | Trema orientalis | −2.34085 | −2.16704 |
| UniRef100_A0A2P5EQB9 | Glycoside hydrolase | Trema orientalis | −4.39941 | −2.1008 |
| UniRef100_A0A2P5EVL2 | LRR domain containing protein | Trema orientalis | −2.32633 | −2.15456 |
| UniRef100_A0A2P5EVZ4 | 4-hydroxyphenylpyruvate | Trema orientalis | −3.49134 | −2.51105 |
| | dioxygenase | | | |
| UniRef100_A0A2P5EXE1 | Laccase | Trema orientalis | −4.23948 | −4.77957 |
| UniRef100_A0A2P5EXQ5 | Cellulose synthase | Trema orientalis | −3.58744 | −2.39827 |
| UniRef100_A0A2P5EYQ8 | Cytochrome P450, E-class, group | Trema orientalis | −2.30612 | −2.65384 |
| | I | | | |
| UniRef100_A0A2P5EYR7 | Xyloglucan | Trema orientalis | −2.04745 | −2.62059 |
| | endotransglucosylase/hydrolase | | | |
| UniRef100_A0A2P5EZT1 | Long-chain-alcohol oxidase | Trema orientalis | −5.05956 | −2.53842 |
| UniRef100_A0A2P5F0U9 | Zinc finger, CCHC-type | Trema orientalis | −3.77724 | −2.06893 |
| | (Fragment) | | | |
| UniRef100_A0A2P5F0Z8 | F-box domain containing protein | Trema orientalis | −3.24318 | −3.24716 |
| UniRef100_A0A2P5F128 | ABC-2 type transporter | Trema orientalis | −2.33389 | −2.29943 |
| UniRef100_A0A2P5F1R0 | Glycoside hydrolase | Trema orientalis | −3.67204 | −3.52535 |
| UniRef100_A0A2P5F1W6 | AP2/ERF transcription factor | Trema orientalis | −2.67506 | −2.6734 |
| UniRef100_A0A2P5F284 | Endoglucanase | Trema orientalis | −4.35661 | −3.14034 |
| UniRef100_A0A2P5F3R8 | Phytocyanin domain containing | Trema orientalis | −2.01835 | −3.27835 |
| | protein | | | |
| UniRef100_A0A2P5F3T0 | Glutamate dehydrogenase | Trema orientalis | −2.12965 | −2.30222 |
| UniRef100_A0A2P5F3U3 | Transmembrane protein | Trema orientalis | −4.72859 | −5.12286 |
| UniRef100_A0A2P5F3Y6 | Pectinesterase | Trema orientalis | −4.26667 | −3.01368 |
| UniRef100_A0A2P5F559 | Cytochrome P | Trema orientalis | −3.90197 | −2.43314 |
| UniRef100_A0A2P5F594 | Amine oxidase | Trema orientalis | −3.32278 | −2.66772 |
| UniRef100_A0A2P5F603 | Glycoside hydrolase | Trema orientalis | −4.15854 | −2.29422 |
| UniRef100_A0A2P5F612 | Glycoside hydrolase (Fragment) | Trema orientalis | −5.04322 | −2.90228 |
| UniRef100_A0A2P5F612 | Glycoside hydrolase (Fragment) | Trema orientalis | −4.02993 | −2.87847 |
| UniRef100_A0A2P5F6K8 | Pectinesterase, catalytic | Trema orientalis | −3.39612 | −2.27546 |
| UniRef100_A0A2P5F7S4 | 1,4-alpha-glucan-branching | Trema orientalis | −4.14186 | −2.08908 |
| | enzyme | | | |
| UniRef100_A0A2P5F8R8 | Cytochrome P450 | Trema orientalis | −4.7672 | −2.01881 |
| UniRef100_A0A2P5F8Y4 | Phytocyanin domain containing | Trema orientalis | −2.68256 | −2.10595 |
| | protein | | | |
| UniRef100_A0A2P5F9L0 | Major latex protein domain | Trema orientalis | −3.64747 | −2.05073 |
| | containing protein | | | |
| UniRef100_A0A2P5FAJ4 | SPX domain containing protein | Trema orientalis | −4.26502 | −2.63769 |
| UniRef100_A0A2P5FAL2 | S-adenosyl-L-methionine- | Trema orientalis | −2.25297 | −3.01839 |
| | dependent methyltransferase | | | |
| UniRef100_A0A2P5FB83 | Proteinase inhibitor | Trema orientalis | −3.88391 | −4.00805 |
| UniRef100_A0A2P5FCR2 | Aldo/keto reductase/potassium | Trema orientalis | −3.79678 | −2.03923 |
| | channel subunit beta (Fragment) | | | |
| UniRef100_A0A2P5FEC6 | Small GTP-binding domain | Trema orientalis | −2.83897 | −2.12515 |
| | containing protein | | | |
| UniRef100_A0A2P5FFP1 | Desiccation-related protein | Trema orientalis | −2.89444 | −3.38466 |
| UniRef100_A0A2P5FFT7 | Bidirectional sugar transporter | Trema orientalis | −3.24285 | −2.58616 |
| | SWEET | | | |
| UniRef100_A0A2P5FG06 | C2-GRAM domain containing | Trema orientalis | −2.52074 | −3.20378 |
| | protein | | | |
| UniRef100_A0A2P5FGM2 | Late embryogenesis abundant | Trema orientalis | −2.66224 | −3.00142 |
| | protein | | | |
| UniRef100_A0A2P5FH06 | Pectinesterase | Trema orientalis | −2.97573 | −3.09771 |
| UniRef100_A0A2P5FHK8 | MYB transcription factor | Trema orientalis | −2.65298 | −2.30378 |
| UniRef100_A0A2P5FI72 | Amidase | Trema orientalis | −2.69109 | −3.36963 |
| UniRef100_A0A2P5FJ20 | Endoglucanase | Trema orientalis | −3.13859 | −3.33578 |
| UniRef100_A0A2P5FJF3 | Cotton fibre protein | Trema orientalis | −3.34498 | −2.78928 |
| UniRef100_A0A2P5FJQ8 | S-adenosyl-L-methionine- | Trema orientalis | −3.15845 | −3.80575 |
| | dependent methyltransferase | | | |
| UniRef100_A0A2P5FJS0 | Non-specific lipid-transfer protein | Trema orientalis | −5.13766 | −2.04144 |
| UniRef100_A0A2P5FK04 | LRR domain containing protein | Trema orientalis | −2.41221 | −4.97443 |
| UniRef100_A0A2P5FKI5 | Hydroxyproline-rich glycoprotein | Trema orientalis | −3.9456 | −3.52003 |
| | family protein | | | |
| UniRef100_A0A2P5FL84 | Proteinase inhibitor 13, Kunitz | Trema orientalis | −3.3113 | −2.40808 |
| | legume | | | |
| UniRef100_A0A2P5FL99 | Hyccin | Trema orientalis | −2.76278 | −2.48838 |
| UniRef100_A0A2P5FND0 | Glycosyl transferase | Trema orientalis | −2.60512 | −2.15794 |
| UniRef100_A0A2P5FNI8 | Carboxypeptidase | Trema orientalis | −2.74002 | −3.5514 |
| UniRef100_A0A2P5FNN9 | Beta-hydroxyacyl-(Acyl-carrier- | fabids | −2.97399 | −3.48138 |
| | protein) dehydratase FabZ | | | |
| UniRef100_A0A2P5FPN5 | Nucleotide-diphospho-sugar | Trema orientalis | −2.74993 | −3.29221 |
| | transferase | | | |
| UniRef100_A0A2P5FPP2 | Phytocyanin domain containing | Trema orientalis | −3.83589 | −3.06171 |
| | protein | | | |
| UniRef100_A0A2P5FR48 | Zinc finger, CCHC-type | Trema orientalis | −3.23177 | −4.04227 |
| UniRef100_A0A2P5FRN4 | Glucose-methanol-choline | Trema orientalis | −4.65028 | −4.70186 |
| | oxidoreductase, N-terminal | | | |
| UniRef100_A0A2P5FRP9 | Glucose-methanol-choline | Trema orientalis | −5.02912 | −2.17694 |
| | oxidoreductase, C-terminal | | | |
| UniRef100_A0A2P5FTA7 | AP endonuclease 1, binding site | Trema orientalis | −4.68886 | −2.13813 |
| | (Fragment) | | | |
| UniRef100_A0A2P5FTH1 | Xanthine dehydrogenase C subunit | Trema orientalis | −3.5846 | −3.9793 |
| | (Fragment) | | | |
| UniRef100_A0A2P5FTH9 | Xanthine dehydrogenase C subunit | Trema orientalis | −3.69762 | −2.69985 |
| UniRef100_A0A2P5FTI4 | Xanthine dehydrogenase C subunit | Trema orientalis | −5.13724 | −3.07836 |
| | (Fragment) | | | |
| UniRef100_A0A2P5FUZ9 | Peroxidase | Trema orientalis | −3.40501 | −3.93175 |
| UniRef100_A0A2P5FVJ8 | Glycosyltransferase | Trema orientalis | −4.17523 | −2.06908 |
| UniRef100_A0A2P5FW38 | Glycoside hydrolase | Trema orientalis | −3.78525 | −3.77282 |
| UniRef100_A0A2P5FWE2 | Homocysteine-binding domain | Trema orientalis | −4.26563 | −2.20043 |
| | containing protein | | | |
| UniRef100_A0A2P5FWP7 | Lipoxygenase | Trema orientalis | −2.43057 | −3.1529 |
| UniRef100_A0A2P5FWU1 | Aspartic peptidase (Fragment) | Trema orientalis | −4.06538 | −3.2045 |
| UniRef100_A0A2P5FX55 | CASP-like protein | Trema orientalis | −5.61344 | −2.23481 |
| UniRef100_A0A2P5FX73 | NAD(P)-binding domain | Trema orientalis | −3.38956 | −2.77502 |
| | containing protein | | | |
| UniRef100_A0A2P5FXU6 | PQ-loop repeat | Trema orientalis | −3.34912 | −3.50384 |
| UniRef100_A0A2P5FXZ8 | Cytochrome P450, E-class, group I | Trema orientalis | −5.55869 | −2.64129 |
| UniRef100_A0A2P5FYH1 | Late embryogenesis abundant | Trema orientalis | −2.21765 | −2.17422 |
| | (LEA) hydroxyproline-rich | | | |
| | glycoprotein family | | | |
| UniRef100_A0A2P5FYJ3 | Trehalose 6-phosphate | Trema orientalis | −2.22961 | −2.36865 |
| | phosphatase | | | |
| UniRef100_A0A2P5G0I1 | Zinc finger, Dof-type | Trema orientalis | −3.6401 | −2.21919 |
| UniRef100_A0A2P5G149 | Branched-chain-amino-acid | Trema orientalis | −3.25978 | −3.2729 |
| | aminotransferase | | | |
| UniRef100_A0A2P5G209 | LL-diaminopimelate | Trema orientalis | −3.44497 | −3.02176 |
| | aminotransferase | | | |
| UniRef100_A0A2P6P5G0 | Putative chromatin remodeling & | Rosa chinensis | −3.15046 | −3.65516 |
| | transcription regulator BTB-POZ | | | |
| | family | | | |
| UniRef100_A0A2R6P5V1 | Exo-beta-D-glucosaminidase | Actinidia chinensis | −4.77692 | −2.92975 |
| | | var. chinensis | | |
| UniRef100_A7WMF5 | Myb factor | Humulus lupulus | −3.38243 | −2.91476 |
| UniRef100_B9TJB3 | Xyloglucan:xyloglucosyl | Ricinus communis | −4.86314 | −2.05954 |
| | transferase, putative | | | |
| UniRef100_F2E7I0 | Predicted protein | Hordeum vulgare | −3.36166 | −2.5537 |
| | | subsp. vulgare | | |
| UniRef100_H6WS87 | Chitinase 3 | Populus canadensis | −2.90437 | −2.68104 |
| UniRef100_J3SDF5 | Ty3/gypsy retrotransposon protein | Beta vulgaris subsp. | −3.91114 | −2.93948 |
| | | vulgaris | | |
| UniRef100_K9JA06 | Xyloglucanase inhibitor 3 | Humulus lupulus | −5.02217 | −3.75738 |
| UniRef100_K9JA99 | Xyloglucanase inhibitor 2 | Humulus lupulus | −5.04573 | −3.079 |
| UniRef100_L7TV16 | Kiwellin | Actinidia arguta | −4.17134 | −2.17256 |
| UniRef100_M4IQQ5 | CCL4 | Humulus lupulus | −5.13874 | −3.23661 |
| UniRef100_M5XGQ8 | Beta-galactosidase | Prunus persica | −4.72053 | −3.55531 |
| UniRef100_Q52H50 | Beta-galactosidase (Fragment) | Carica papaya | −4.83944 | −2.91349 |
| UniRef100_Q6L3Q0 | Polyprotein, putative | Solarium demissum | −4.52252 | −2.09211 |
| UniRef100_Q7XPF2 | OSJNBa0060N03.9 protein | Oryza sativa subsp. | −2.25616 | −2.16979 |
| | | japonica | | |
| UniRef100_Q8RVK9 | Naringenin-chalcone synthase | Cannabis sativa | −3.053 | −2.27014 |
| UniRef100_Q9AVC7 | Plasma membrane H+-ATPase | Vallisneria gigantea | −2.03414 | −3.95653 |
| | (Fragment) | | | |
| UniRef100_Q9AVH2 | Putative senescence-associated | Pisum sativum | −2.63349 | −2.61696 |
| | protein (Fragment) | | | |
| UniRef100_UPI0002C2EBAF | glu S.griseus protease inhibitor- | Fragaria vesca subsp. | −4.36113 | −3.31667 |
| | like | vesca | | |
| UniRef100_UPI00046DD824 | laccase-15-like | Prunus mume | −3.42301 | −3.32623 |
| UniRef100_UPI00046E2595 | beta-galactosidase-like | Prunus mume | −4.70916 | −2.89539 |
| UniRef100_UPI000498E3OC | epidermis-specific secreted | Malus domestica | −5.08037 | −2.40944 |
| | glycoprotein EPl-like | | | |
| UniRef100_UPI00049913B6 | phosphate transporter PHO1 | Malus domestica | −2.24126 | −2.0319 |
| UniRef100_UPI0004991CB1 | cyanogenic beta-glucosidase-like | Malus domestica | −2.41232 | −3.07917 |
| UniRef100_UPI0005115765 | primary amine oxidase-like | Pyrus × bretschneideri | −2.64495 | −2.10781 |
| UniRef100_UPI0005116EA6 | glutathione reductase, cytosolic | Pyrus × bretschneideri | −4.00244 | −2.69981 |
| UniRef100_UPI000511B354 | vicianin hydrolase-like | Pyrus × bretschneideri | −2.95458 | −3.3508 |
| UniRef100_UPI00053BBD5B | beta-galactosidase 4 | Camelina sativa | −4.69232 | −3.17262 |
| UniRef100_UPI00057A7COA | beta-carotene 3-hydroxylase 2, | Elaeis guineensis var. | −3.4174 | −2.19506 |
| | chloroplastic-like | tenera | | |
| UniRef100_UPI00064DAD47 | extensin-like | Erythranthe guttata | −4.71951 | −2.03685 |
| UniRef100_UPI00077E4CB6 | geraniol 8-hydroxylase-like | Ziziphus jujuba | −2.53423 | −2.23604 |
| UniRef100_UPI00077EAC50 | endochitinase EP3-like isoform XI | Ziziphus jujuba | −2.69435 | −5.02058 |
| UniRef100_UPI00077EAE6B | methionine gamma-lyase | Ziziphus jujuba | −2.17115 | −2.21583 |
| UniRef100_UPI00077EC6EE | beta-galactosidase 8-like | Ziziphus jujuba | −2.9149 | −3.44892 |
| UniRef100_UPI00077ECA71 | cytochrome P450 CYP82D47-like | Ziziphus jujuba | −5.9607 | −2.87481 |
| UniRef100_UPI0007AEF614 | myb-related protein 308 | Arachis ipaensis | −2.52482 | −2.20404 |
| UniRef100_UPI0007ECA6DB | glutamate-cysteine ligase, | Malus domestica | −2.66227 | −2.18983 |
| | chloroplastic-like isoform X3 | | | |
| UniRef100_UPI0008485E4A | pectinesterase 2 | Theobroma cacao | −3.75547 | −3.66772 |
| UniRef100_UPI000848C6D2 | vignain | Theobroma cacao | −4.41271 | −5.50247 |
| UniRef100_UPI000901F9F1 | gibberellin 3-beta-dioxygenase 1- | Ipomoea nil | −4.8512 | −2.2901 |
| | like | | | |
| UniRef100_UPI000A2B650A | transcription repressor MYB5-like | Arachis ipaensis | −3.02193 | −3.0565 |
| UniRef100_UPI0OOB3EE3B3 | putative beta-D-xylosidase isoform | Herrania umbratica | −5.03292 | −2.14908 |
| | X1 | | | |
| UniRef100_UPI0OOB3F1AEF | LOB domain-containing protein | Herrania umbratica | −2.57899 | −3.44373 |
| | 18 | | | |
| UniRef100_UPI000B3F2392 | myb-related protein 308-like | Herrania umbratica | −2.92467 | −2.02195 |
| UniRef100_UPI000B7B83D0 | probable low-specificity L- | Prunus avium | −3.30205 | −3.60234 |
| | threonine aldolase 1 isoform XI | | | |
| UniRef100_UPI000B8D00F4 | proteinase inhibitor | Carica papaya | −4.04394 | −3.74082 |
| UniRef100_UPI000B935E6B | beta-galactosidase-like | Momordica charantia | −4.11316 | −2.0078 |
| UniRef100_UPI000C05562E | MLO-like protein 6 | Durio zibethinus | −3.10932 | −3.17357 |
| UniRef100_UPI000C21BC57 | AAA-ATPase ASD, | Cucurbita maxima | −6.05426 | −2.68343 |
| | mitochondrial-like | | | |
| UniRef100_UPI000C2256A1 | probable acyl-activating enzyme 5, | Cucurbita maxima | −3.96196 | −3.16228 |
| | peroxisomal | | | |
| UniRef100_UPI000CE24524 | beta-D-xylosidase 1-like | Quercus suber | −6.08724 | −2.61184 |
| UniRef100_UPI0OOCEDOAFE | epidermis-specific secreted | Morus notabilis | −5.26672 | −2.81326 |
| | glycoprotein EPl | | | |
| UniRef100_UPI000CED165A | chaicone synthase-like | Morus notabilis | −2.5455 | −2.34851 |
| UniRef100_UPI000CED1BDB | branched-chain-amino-acid | Morus notabilis | −3.28355 | −3.51423 |
| | aminotransferase 2, chloroplastic | | | |
| UniRef100_UPI000CED2196 | inositol oxygenase | Morus notabilis | −2.99002 | −4.8278 |
| UniRef100_UPI000CED4F53 | (R)-mandelonitrile lyase 1 | Morus notabilis | −3.6041 | −2.04721 |
| UniRef100_UPI000CED5A8O | cytochrome P450 71 Dll isoform | Morus notabilis | −2.68561 | −3.79023 |
| | XI | | | |
| UniRef100_UPI000CED5C46 | (R)-mandelonitrile lyase 1 | Morus notabilis | −4.72628 | −2.23673 |
| UniRef100_UPI000CED5DA6 | GDSL esterase/lipase 1-like | Morus notabilis | −2.36408 | −4.10876 |
| UniRef100_UPI000CED6849 | probable glucan endo-l,3-beta- | Morus notabilis | −4.94268 | −2.0419 |
| | glucosidase BG4 | | | |
| UniRef100_UPI000CED7248 | cytochrome P450 71 Dll-like | Morus notabilis | −2.29882 | −2.58306 |
| UniRef100_UPI000CED74C0 | protein IQ-DOMAIN 14-like | Morus notabilis | −2.44176 | −4.44835 |
| UniRef100_UPI000D629444 | cytochrome P450 87A3-like | Ziziphus jujuba | −3.75763 | −2.39058 |
| UniRef100_V4U3F0 | Glutamate dehydrogenase | Citrus Clementina | −2.65542 | −3.30073 |
| UniRef100_W9QJ91 | Laccase | Morus notabilis | −3.53401 | −2.61651 |
| UniRef100_W9QZB4 | Cellulose synthase-like protein E6 | Morus notabilis | −3.43735 | −3.10797 |
| UniRef100_W9R6G8 | Beta-D-xylosidase 1 | Morus notabilis | −4.08929 | −2.61731 |
| UniRef100_W9R7T4 | Extended synaptotagmin-2 | Morus notabilis | −3.39525 | −2.86248 |
| UniRef100_W9RHK9 | L-allo-threonine aldolase | Morus notabilis | −3.49931 | −3.05397 |
| UniRef100_W9RKZ8 | Laccase | Morus notabilis | −3.19656 | −3.05518 |
| UniRef100_W9RMB5 | Patatin | Morus notabilis | −2.82499 | −2.18333 |
| UniRef100_W9RPG3 | ABC transporter G family member | Morus notabilis | −2.43967 | −2.59114 |
| | 31 | | | |
| UniRef100_W9RZW3 | Prostaglandin G/H synthase 1 | Morus notabilis | −2.68393 | −2.10988 |
| UniRef100_W9S1D3 | Dihydrolipoamide | Morus notabilis | −2.78208 | −2.19231 |
| | acetyltransferase component of | | | |
| | pyruvate dehydrogenase complex | | | |
| UniRef100_W9S3X0 | Alpha-aminoadipic semialdehyde | Morus notabilis | −3.07611 | −2.74561 |
| | synthase | | | |
| UniRef100_W9SID6 | Polygalacturonase inhibitor | Morus notabilis | −2.32289 | −2.66516 |
| UniRef100_W9SN58 | Amine oxidase | Morus notabilis | −2.32093 | −3.49704 |
| UniRef100_W9SNF9 | Epidermis-specific secreted | Morus notabilis | −5.54215 | −2.86378 |
| | glycoprotein EP1 | | | |
| UniRef100_W9SVJ3 | Cytochrome P450 93A1 | Morus notabilis | −2.71326 | −2.99276 |

TABLE 13
Differentially expressed genes across developmental Stage 3 in trichomes
and flowers with log2FoldChange and UniRef100 annotation.

| | | | Stage2 v Stage3 | |
|---|---|---|---|---|
| UniRef100 Annotation | Name | Taxonomy | log2FoldChange | Tissue |
| UniRef100_A0A1E1XPA8 | Putative eremothecium ashbyi | Arthropoda | −5.643053546 | Trichome |
| | 26s rrna sequence (Fragment) | | | |
| UniRef100_A0A2P5ARP8 | Tetratricopeptide-like helical | Parasponia | −2.255618984 | Trichome |
| | domain containing protein | andersonii | | |
| UniRef100_A0A2P5BVU6 | Short-chain | Parasponia | −2.26869245 | Trichome |
| | dehydrogenase/reductase | andersonii | | |
| UniRef100_A0A2P5DVB1 | Bidirectional sugar transporter | Parasponia | −2.173349146 | Trichome |
| | SWEET | andersonii | | |
| UniRef100_A0A2P5F8H7 | Cytochrome P450, E-class, | Trema orientalis | −2.457673246 | Trichome |
| | group I | | | |
| UniRef100_A0A2P5FWA6 | Chlorophyll A-B binding protein | Trema orientalis | −2.057798134 | Flower |
| UniRef100_A0A2P5FWA6 | Chlorophyll A-B binding protein | Trema orientalis | −2.66156876 | Trichome |
| UniRef100_UPI000CED1130 | laccase-4 | Mortis notabilis | −2.579580089 | Flower |
| UniRef100_UPI000D62B9FB | laccase-4-like | Ziziphus jujuba | −2.690729105 | Flower |
| UniRef100_W9QZR2 | Citrate synthase | Mortis notabilis | −3.075646927 | Flower |

TABLE 14
Differentially expressed genes across developmental Stage 4 in trichomes
and flowers with log2FoldChange and UniRef100 annotation.

| | | | Stage3 versus | |
|---|---|---|---|---|
| | | | Stage4 | |
| UniRef100 Annotation | Name | Taxonomy | log2FoldChange | Tissue |
| UniRef100_A0A061S8Z6 | Cytochrome p450 liketbp | Tetraselmis sp. | −2.513004899 | Flower |
| | | GSL018 | | |
| UniRef100_A0A072UJ16 | Signal anchor, putative | Medicago | −2.164231131 | Flower |
| | | truncatula | | |
| UniRef100_A0A077ZKY0 | Cell wall-associated hydrolase | Trichuris trichiura | −2.984278847 | Flower |
| UniRef100_A0A078I0S5 | BnaC08g47040D protein | Brassica napus | −2.202216206 | Flower |
| UniRef100_A0A078I3U3 | BnaCnng12640D protein | Brassica napus | −2.448963556 | Flower |
| UniRef100_A0A078I637 | BnaCnng13060D protein | Brassica napus | −2.080835583 | Flower |
| UniRef100_A0A0L1KIP1 | Cell wall-associated hydrolase | Candidatus | −3.597431331 | Flower |
| | | Burkholderia | | |
| | | brachyanthoides | | |
| UniRef100_A0A0N0I3T1 | Cell wall-associated hydrolase | Geobacillus sp. | −6.085524536 | Flower |
| | | BCO2 | | |
| UniRef100_A0A0P6IXY1 | Daphnid bacterial-ribosomal- | Daphnia magna | −4.880130673 | Flower |
| | RNA-like, possible HGT | | | |
| UniRef100_A0A0V0GJC4 | Putative ovule protein | ellular organisms | −2.504995467 | Flower |
| | (Fragment) | | | |
| UniRef100_A0A0V0GPW7 | Putative ovule protein | Solanum | −2.96681215 | Flower |
| | | chacoense | | |
| UniRef100_A0A0V0GQD1 | Putative ovule protein | Solanum | −2.175405779 | Flower |
| | (Fragment) | chacoense | | |
| UniRef100_A0A0V0GW22 | Putative ovule protein | Solanum | −2.566247437 | Flower |
| | (Fragment) | chacoense | | |
| UniRef100_A0A1V0QSF3 | Terpene synthase | Cannabis sativa | −2.035223853 | Flower |
| UniRef100_A0A1V0QSF6 | Terpene synthase | Cannabis sativa | −2.08810414 | Flower |
| UniRef100_A0A1V0QSH9 | HDR (Fragment) | Cannabis sativa | −2.173258452 | Flower |
| UniRef100_A0A288W7D2 | Orf187 | Pentapetalae | −2.325864923 | Flower |
| UniRef100_A0A2G2UWV3 | Regulator of rDNA | Capsicum | −2.072463718 | Flower |
| | transcription protein 15 | baccatum | | |
| UniRef100_A0A2G2UXT1 | Regulator of rDNA | Capsicum | −3.287408717 | Flower |
| | transcription protein 15 | baccatum | | |
| UniRef100_A0A2G2UZB3 | Protein TAR1 | Capsicum | −2.672088701 | Flower |
| | | baccatum | | |
| UniRef100_A0A2G2V0X3 | Protein TAR1 | Capsicum | −4.433190258 | Flower |
| | | baccatum | | |
| UniRef100_A0A2K3PPZ3 | Retrotransposon-related protein | Trifolium pratense | −2.014524214 | Flower |
| UniRef100_A0A2M4CVV1 | Hipothetical protein | Anopheles darlingi | −5.155060698 | Flower |
| | (Fragment) | | | |
| UniRef100_A0A2M4CWH3 | Hipothetical protein | Anopheles darlingi | −3.621383197 | Flower |
| | (Fragment) | | | |
| UniRef100_A0A2P4HR67 | Gibberellin 3-beta-dioxygenase | Quercus suber | −2.163504153 | Flower |
| | 1 | | | |
| UniRef100_A0A2P4J0X6 | Chlorophyllide a oxygenase, | Quercus suber | −2.03436965 | Flower |
| | chloroplastic | | | |
| UniRef100_A0A2P4JE61 | Protein kinase pinoid | Quercus suber | −2.123653022 | Flower |
| UniRef100_A0A2P5AG05 | Cytochrome P450, E-class, | Trema orientalis | −2.617880589 | Flower |
| | group I | | | |
| UniRef100_A0A2P5API2 | Tetratricopeptide-like helical | Trema orientalis | −2.084045242 | Flower |
| | domain containing protein | | | |
| UniRef100_A0A2P5AZ66 | Transferase | Parasponia | −2.376393919 | Trichome |
| | | andersonii | | |
| UniRef100_A0A2P5B371 | Glycoside hydrolase | Parasponia | −2.652276226 | Trichome |
| | | andersonii | | |
| UniRef100_A0A2P5BJZ7 | Oxoglutarate/iron-dependent | Trema orientalis | −2.036410403 | Flower |
| | dioxygenase (Fragment) | | | |
| UniRef100_A0A2P5BWV8 | Basic helix-loop-helix | Parasponia | −2.042768077 | Trichome |
| | transcription factor | andersonii | | |
| UniRef100_A0A2P5CLL7 | Glycoside hydrolase | Parasponia | −2.290170452 | Flower |
| | | andersonii | | |
| UniRef100_A0A2P5CYC3 | Pectinesterase | Parasponia | −2.428588106 | Flower |
| | | andersonii | | |
| UniRef100_A0A2P5D1P2 | Floricaula/leafy protein | Parasponia | −2.642857039 | Trichome |
| | | andersonii | | |
| UniRef100_A0A2P5DP00 | Transferase | Trema orientalis | −2.558447368 | Flower |
| UniRef100_A0A2P5DZ03 | Glycoside hydrolase | Parasponia | −2.233123635 | Flower |
| | | andersonii | | |
| UniRef100_A0A2P5E4Y3 | Cell wall protein | Parasponia | −6.219861348 | Trichome |
| | | andersonii | | |
| UniRef100_A0A2P5E6G3 | E3 ubiquitin-protein ligase | Trema orientalis | −2.60263322 | Flower |
| | SIN-like | | | |
| UniRef100_A0A2P5EFV8 | Calmodulin-binding family | Trema orientalis | −2.197354268 | Trichome |
| | protein | | | |
| UniRef100_A0A2P5EGL6 | Non-specific serine/threonine | Trema orientalis | −2.086123151 | Flower |
| | protein kinase | | | |
| UniRef100_A0A2P5EQA8 | Glycoside hydrolase | Trema orientalis | −2.251089162 | Flower |
| UniRef100_A0A2P5EQV0 | Cytochrome P450, E-class, | Trema orientalis | −2.380323064 | Trichome |
| | group I | | | |
| UniRef100_A0A2P5EZT1 | Long-chain-alcohol oxidase | Trema orientalis | −2.010248992 | Flower |
| UniRef100_A0A2P5FH06 | Pectinesterase | Trema orientalis | −2.950830968 | Flower |
| UniRef100_A0A2P5FKI5 | Hydroxyproline-rich | Trema orientalis | −2.040148718 | Flower |
| | glycoprotein family protein | | | |
| UniRef100_A0A2R6P250 | Altered inheritance of | Actinidia chinensis | −3.001623304 | Flower |
| | mitochondria protein | var. chinensis | | |
| UniRef100_A0A2R6QID4 | Aldehyde dehydrogenase | Actinidia chinensis | −2.12822237 | Flower |
| | family 16 member like | var. chinensis | | |
| | (Fragment) | | | |
| UniRef100_A0A2S1PH03 | RNA-directed RNA | Rubber dandelion | −21.61715989 | Trichome |
| | polymerase | latent virus 1 | | |
| UniRef100_B0ZB57 | Probable O-methyltransferase 3 | Humulus lupulus | −2.397212885 | Flower |
| UniRef100_B6SCF4 | Myrcene synthase, | Humulus lupulus | −3.431831878 | Flower |
| | chloroplastic | | | |
| UniRef100_D6K2G4 | Cell wall-associated hydrolase | Streptomyces sp. | −5.619291107 | Flower |
| | | e14 | | |
| UniRef100_Q6T4Q2 | Putative senescence-associated | Pyrus communis | −2.266855196 | Flower |
| | protein (Fragment) | | | |
| UniRef100_Q8RVK9 | Naringenin-chalcone synthase | Cannabis sativa | −2.390205639 | Flower |
| UniRef100_T2DPD7 | Cell wall-associated hydrolase | Phaseolus vulgaris | −2.332294946 | Flower |
| UniRef100_UPI000CD87AOF | probable serine/threonine | Lactuca sativa | −2.440174642 | Flower |
| | protein kinase IREH1 | | | |
| UniRef100_UPI000CED1C21 | germin-like protein subfamily | Morus notabilis | −3.525739811 | Flower |
| | 1 member 17 | | | |
| UniRef100_W9SBV7 | Metal transporter Nramp5 | Morus notabilis | −2.071441248 | Flower |

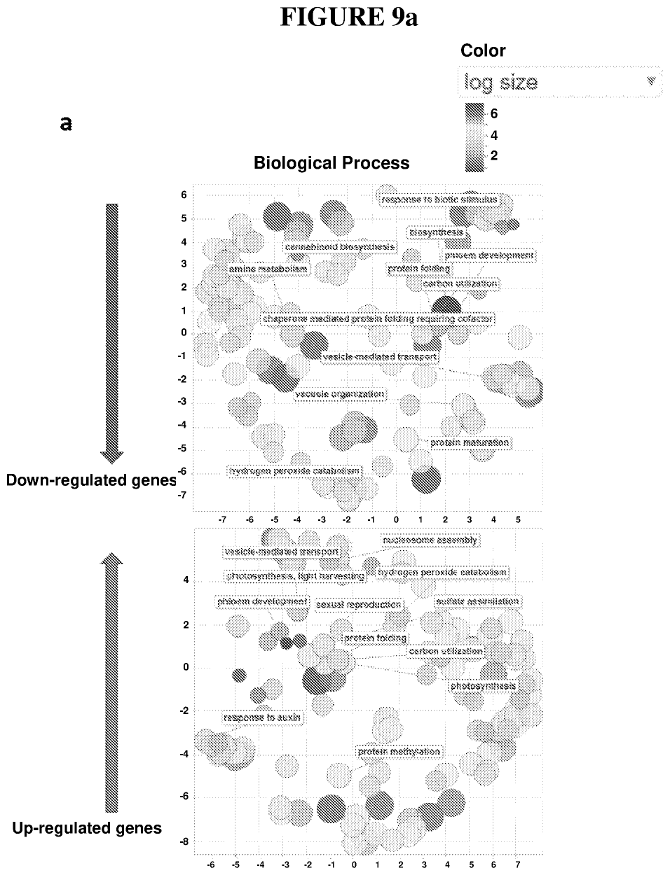

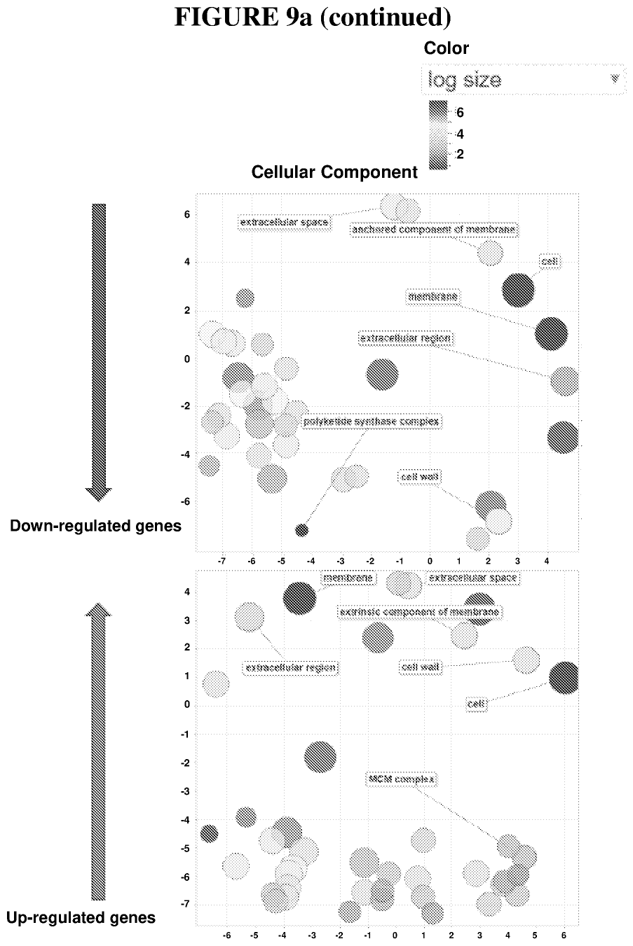

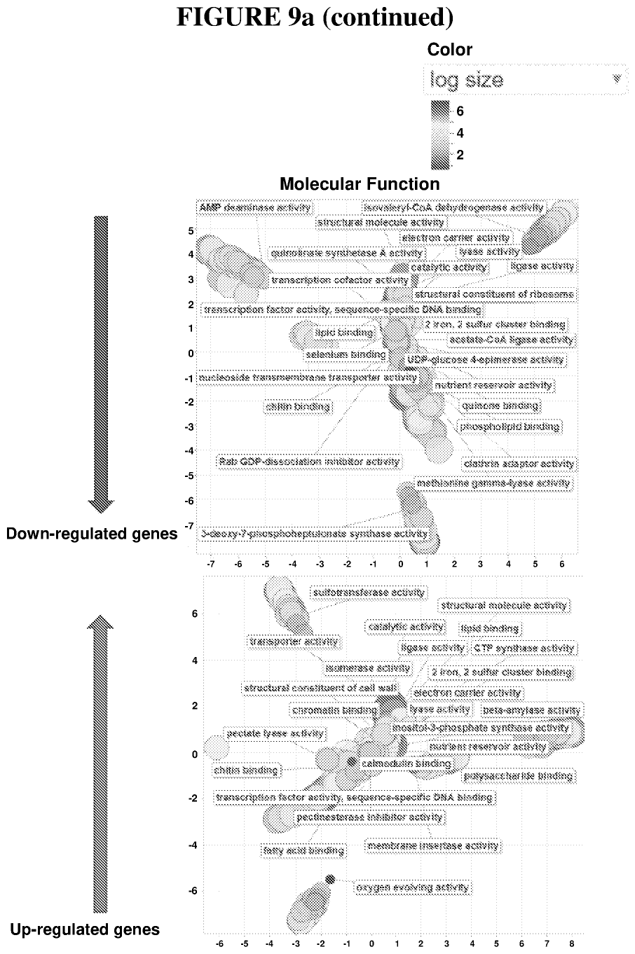

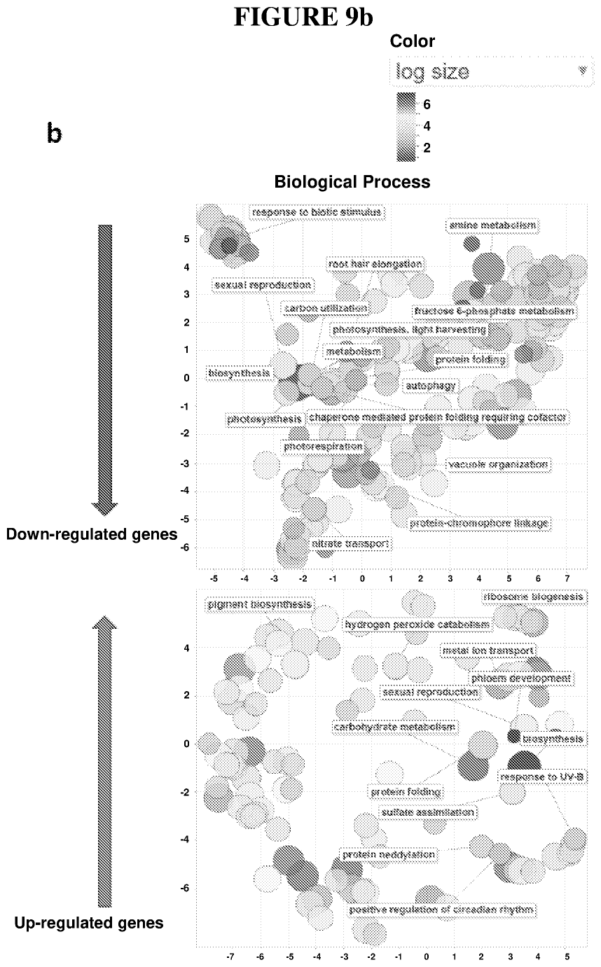

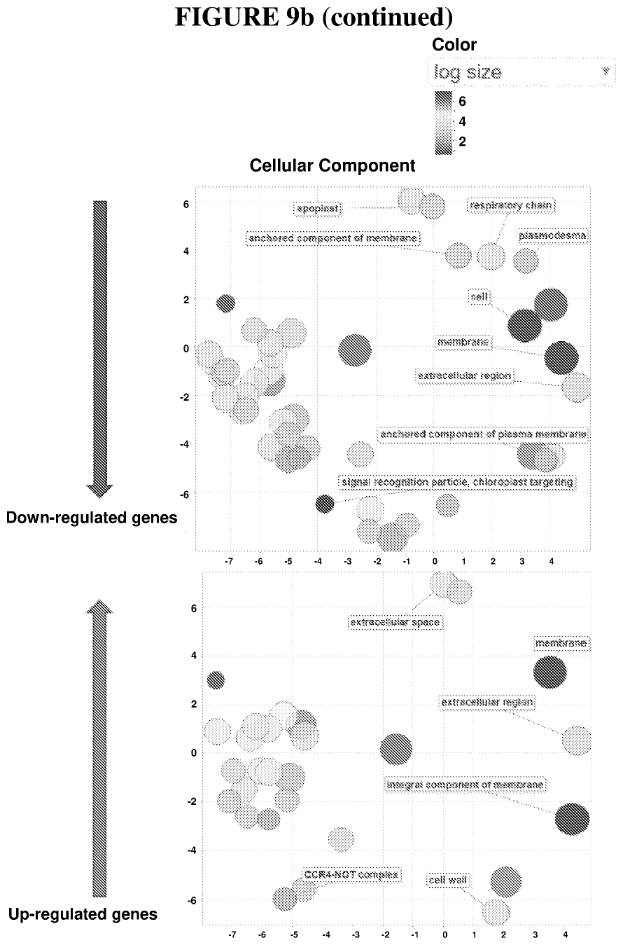

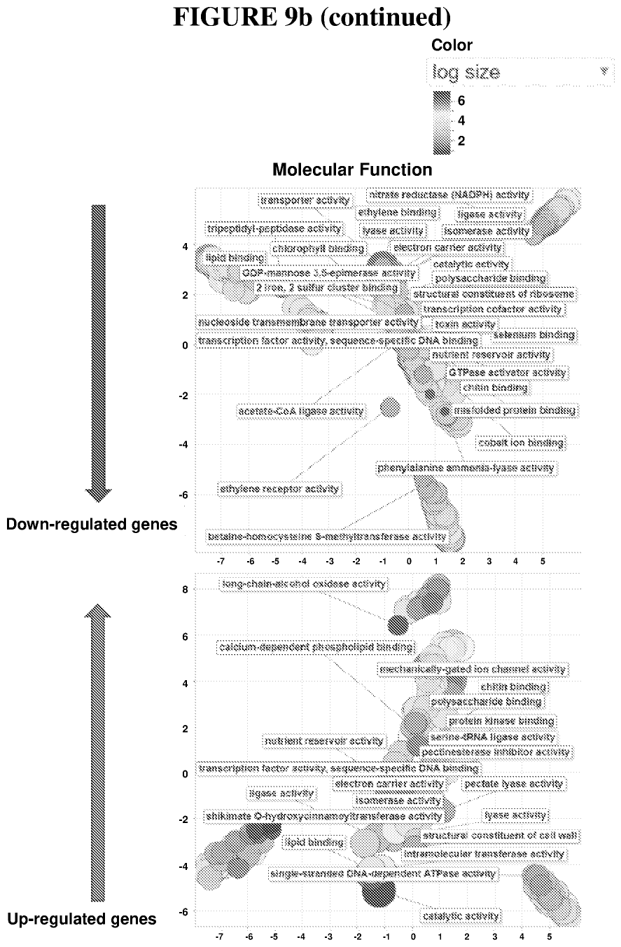

The number of differentially expressed genes between Stages 1 when compared to Stage 4 were found to be maximum and these genes were further categorised functionally based on their GO term (FIG. 9). The majority of the enriched genes in each comparison were attributed to a functional category, in which the most frequent categories were “catalytic activity” and “binding”; followed by biological and cellular categories. The GO category for biological process revealed that the number of enriched genes in the two types of “metabolic process” and “cellular process” was the largest. The most prevalent GO categories for cellular component included “membrane” and “membrane part”.

These results were further refined by comparing the expression of Stage 1 and Stage 4 to identify a subset of transcripts that are significantly differentially expressed between Stage 1 and Stage 4 female flower and trichome tissues, as detailed in Table 15.

TABLE 15
Significantly differentially expressed transcripts
associated with female flower development.

| | | Trichome_S1 v S4 |
|---|---|---|
| Transcript ID | Gene Product | (log₂FC) |
| Cannbio_031223 | THCA synthase (Fragment) | −4.301237704 |
| Cannbio_024022 | THCA synthase (Fragment) | 4.126527581 |
| Cannbio_009678 | Truncated THCA synthase | −2.593755565 |
| Cannbio_039738 | Polyketide synthase 3 | −2.0086964 |
| Cannbio_014959 | Tetrahydrocannabinolic acid synthase (Fragment) | −2.318544817 |
| Cannbio_036667 | HDR (Fragment) | −3.333180775 |
| Cannbio_041839 | HDR (Fragment) | −2.931934104 |
| Cannbio_037023 | HDR (Fragment) | −2.731041122 |
| Cannbio_034678 | HDR (Fragment) | −2.305917442 |
| Cannbio_039063 | HDR (Fragment) | −2.258724797 |
| Cannbio_004871 | HDR (Fragment) | −2.065770153 |
| Cannbio_041521 | HDR (Fragment) | −2.060653618 |
| Cannbio_038684 | HDR (Fragment) | −2.007859466 |
| Cannbio_038698 | HDR (Fragment) | −2.006963312 |
| Cannbio_032875 | Fatty acid desaturase 2 | −2.132184617 |
| Cannbio_006213 | Fatty acid desaturase | −2.885792493 |
| Cannbio_010956 | Fatty acid desaturase | −2.225929584 |
| Cannbio_024800 | Fatty acid desaturase | −3.709316864 |
| Cannbio_063081 | Fatty acid desaturase | −3.684324945 |
| Cannbio_022360 | Fatty acid desaturase | −3.592160994 |
| Cannbio_029003 | Fatty acid desaturase | −2.804167862 |
| Cannbio_005992 | Delta-12 fatty acid desaturase allele 2 | −2.263800008 |
| Cannbio_001610 | Omega-6 fatty acid desaturase | −3.333455927 |
| Cannbio_039875 | Delta(12)-acyl-lipid-desaturase | −2.934791378 |
| Cannbio_047069 | FA_desaturase domain-containing protein/DUF3474 | −3.341009941 |
| | domain-containing protein | |
| Cannbio_031843 | Fatty acid desaturase 2-1 | −2.189089245 |
| Cannbio_042452 | Delta12-oleic acid desaturase | −3.114203616 |
| Cannbio_047742 | Delta12-oleic acid desaturase | −2.949309443 |
| Cannbio_042391 | Delta12-oleic acid desaturase | −2.638385363 |
| Cannbio_015972 | Delta 12 desaturase | −3.627654552 |
| Cannbio_018959 | Delta 12 desaturase | −2.237177072 |
| Cannbio_045108 | Delta 12 | −2.156049138 |
| Cannbio_048952 | Delta 12 desaturase | −2.000202358 |
| Cannbio_042165 | Delta 12-oleate desaturase (Fragment) | −2.279920243 |
| Cannbio_034132 | Delta(12)-acyl-lipid-desaturase-like | −2.94724261 |
| Cannbio_047604 | Omega-6 fatty acid desaturase, endoplasmic reticulum | −3.077058551 |
| | isozyme 1-like | |
| Cannbio_047523 | Omega-6 fatty acid desaturase, endoplasmic reticulum | −3.253418667 |
| | isozyme | |
| Cannbio_027313 | Omega-6 fatty acid desaturase, endoplasmic reticulum | −3.53996223 |
| | isozyme 2 | |
| Cannbio_027028 | Omega-6 fatty acid desaturase, endoplasmic reticulum | −2.891587531 |
| | isozyme 2 | |
| Cannbio_044281 | 3-hydroxy-3-methylglutaryl coenzyme A reductase | 2.004953095 |
| | (Fragment) | |
| Cannbio_046662 | 4-hydroxy-3-methylbut-2-enyl diphosphate reductase | −3.817938512 |
| Cannbio_036703 | 4-hydroxy-3-methylbut-2-enyl diphosphate reductase | −3.121741924 |
| Cannbio_036966 | 4-hydroxy-3-methylbut-2-enyl diphosphate reductase | −2.366497091 |
| Cannbio_001432 | 4-hydroxy-3-methylbut-2-enyl diphosphate reductase | −2.560894233 |
| Cannbio_058668 | (E,E)-geranyllinalool synthase | 3.807482193 |
| Cannbio_012536 | Terpene cyclase/mutase family member | 3.629080582 |
| Cannbio_018129 | Terpene synthase | −3.270318139 |
| Cannbio_046296 | Terpene synthase | −3.141856947 |
| Cannbio_012506 | Terpene synthase | −2.97778812 |
| Cannbio_043287 | Terpene synthase | −2.892100066 |
| Cannbio_004639 | Terpene synthase | −2.82678195 |
| Cannbio_006183 | Terpene synthase | −3.053218073 |
| Cannbio_033257 | Terpene synthase | −2.747457741 |
| Cannbio_033443 | Terpene synthase | −2.381311837 |
| Cannbio_037451 | Terpene synthase | −2.324734759 |
| Cannbio_042088 | Terpene synthase | −2.172316464 |
| Cannbio_016394 | Terpene synthase | −2.817108137 |
| Cannbio_019445 | (−)-limonene synthase, chloroplastic | −2.530694295 |
| Cannbio_037653 | (−)-limonene synthase, chloroplastic | −2.347883683 |
| Cannbio_036960 | (−)-limonene synthase, chloroplastic | −2.298878454 |
| Cannbio_031882 | (+)-alpha-pinene synthase, chloroplastic | −2.485716464 |
| Cannbio_015144 | Germacrene-A synthase | −2.281988842 |
| Cannbio_049822 | Lupeol synthase | 3.05087166 |
| Cannbio_012638 | Vinorine synthase | −4.397640346 |
| Cannbio_033299 | Isoform 2 of MADS-box protein agl42 | −2.477657691 |
| Cannbio_029262 | Agamous-like MADS-box protein AGL11 | 2.152624918 |
| Cannbio_014948 | MADS-box transcription factor | −2.176563373 |
| Cannbio_028894 | MADS-box transcription factor | 2.102026245 |
| Cannbio_043417 | MADS-box transcription factor | 2.121388659 |
| Cannbio_043906 | MADS-box transcription factor | 3.359931392 |
| Cannbio_013615 | MADS-box transcription factor | −2.904772808 |
| Cannbio_047696 | MADS-box transcription factor | 2.095209814 |

Quantitative reverse transcription polymerase chain reaction (qRT-PCR) analysis revealed that all the genes exhibited similar expression patterns in qRT-PCR as observed in the RNA-Seq data (Table 16). A high proportion of the transcripts (17 out of 20) had a correlation coefficient of ≥0.96. The remaining three transcripts displayed slight discordant outcome with Pearson's correlation coefficient ranging between 0.93 and 0.94.

Taken together, these data enable methods for determining the development stage of a female cannabis plant inflourescence. In particular, the differential expression of genes encoding cannabinoid synthesis protein, terpene synthesis protein, MEP pathway protein, MEV pathway protein and MADs box floral initiation transcription factor can be used to determine the developmental stage of a female cannabis plant inflourescence.

TABLE 16
Expression profiles and correlation of selected transcripts
obtained from qRT-PCR and RNA-Seq from different tissues.

| | | | | | Female Flower | | Female Flower | | |
|---|---|---|---|---|---|---|---|---|---|
| | Root | | Leaf | | S1 | | S4 | | Pearson's |
| | qRT- | RNA- | qRT- | RNA- | qRT- | RNA- | qRT- | RNA- | correlation |
| Transcript | PCR | Seq | PCR | Seq | PCR | Seq | PCR | Seq | coefficient |
| 000799 | 1.48 | 11623.00 | 0.00 | 0.00 | 0.00 | 2.17 | 0.00 | 56.00 | 1.00 |
| 013596 | 0.89 | 2287.50 | 0.00 | 1.00 | 0.00 | 2.83 | 0.00 | 20.50 | 1.00 |
| 033634 | 1.04 | 3057.00 | 0.00 | 0.00 | 0.00 | 0.00 | 0.00 | 23.00 | 1.00 |
| 035851 | 0.00 | 0.00 | 0.22 | 117.60 | 0.00 | 7.00 | 0.00 | 22.50 | 0.99 |
| 041591 | 0.04 | 2790.00 | 0.00 | 0.20 | 0.00 | 0.00 | 0.00 | 29.67 | 1.00 |
| 044988 | 0.00 | 0.00 | 2.44 | 175.60 | 0.00 | 49.33 | 0.00 | 26.50 | 0.97 |
| 038927 | 0.00 | 1.00 | 1.72 | 436.20 | 0.43 | 134.00 | 0.15 | 83.33 | 0.99 |
| 050434 | 0.00 | 16.50 | 0.00 | 3.40 | 0.00 | 6.33 | 0.41 | 450.50 | 1.00 |
| 060043 | 0.00 | 2.50 | 0.00 | 37.60 | 0.00 | 75.50 | 0.01 | 3692.50 | 0.96 |
| 051181 | 0.00 | 3.00 | 0.00 | 49.00 | 0.00 | 9.17 | 0.00 | 423.83 | 0.94 |
| 056951 | 0.00 | 15.50 | 0.00 | 37.40 | 0.01 | 196.50 | 0.09 | 2364.17 | 1.00 |
| 009678 | 0.00 | 0.00 | 0.00 | 9.20 | 0.13 | 19.00 | 1.50 | 217.50 | 1.00 |
| 001610 | 0.00 | 11.50 | 0.00 | 20.80 | 0.52 | 175.33 | 5.55 | 1937.17 | 1.00 |
| 042452 | 0.00 | 10.50 | 0.00 | 12.00 | 0.26 | 132.67 | 2.57 | 1423.00 | 1.00 |
| 047069 | 0.00 | 43.00 | 0.00 | 92.80 | 0.20 | 744.67 | 1.51 | 7790.00 | 1.00 |
| 047604 | 0.12 | 26.50 | 0.13 | 60.60 | 1.03 | 702.00 | 9.04 | 6234.83 | 1.00 |
| 027313 | 0.00 | 1.00 | 0.00 | 4.80 | 0.64 | 34.67 | 5.76 | 292.00 | 1.00 |
| 015972 | 0.00 | 28.00 | 0.25 | 447.60 | 0.38 | 988.83 | 0.78 | 6329.17 | 0.93 |
| 026293 | 0.00 | 54.50 | 0.00 | 7.20 | 0.23 | 1047.00 | 0.00 | 49.00 | 1.00 |
| 063731 | 0.00 | 239.50 | 0.00 | 23.00 | 0.01 | 615.17 | 0.00 | 21.83 | 0.94 |

### Example 4

### Expression Analysis of Genes Involved in Terpene and Cannabinoid Biosynthesis

BLASTN searches against the genes involved in terpene synthesis identified 124 transcripts from the MEP pathway, 69 transcripts from the MEV pathway and 24 transcripts as prenyltransferases from the current assembly. A total of 136 transcripts were identified to represent the cannabis TPS out of which TPS1FN was found to be the most abundant in the current assembly followed by TPS8FN, TPS2FN and TPS3FN. In addition, a total of 30 transcripts were identified as THCAS or cannabidiolic acid synthase-like 1 (CBDAS-like 1) or CBDAS based on the annotation of similarity results to UniRef100 database. A summary of the genes identified is detailed in Table 17.

TABLE 17
Terpene and cannabinoid synthesis genes identified by BLASTN.

| | | | Developmental | |
|---|---|---|---|---|
| Reference | Name | Gene ID | Stage | Tissue |
| KY014553.1 | Cannabis sativa HMGR2 mRNA, partial cds | HMGR2 | 1, 2 | Flower/ |
| | | | | Trichome |
| KY014554.1 | Cannabis sativa isolate Finola_TPS7 terpene | TPS7FN | 4 | Flower/ |
| | synthase mRNA, complete cds | | | Trichome |
| KY014555.1 | Cannabis sativa isolate Finola_TPS9 terpene | TPS9FN | 4 | Root/Shoot |
| | synthase mRNA, complete cds | | | |
| KY014556.1 | Cannabis sativa isolate Finola_TPS8 terpene | TPS8FN | 4 | Flower/ |
| | synthase mRNA, complete cds | | | Trichome |
| KY014557.1 | Cannabis sativa isolate Finola_TPS1 terpene | TPS1FN | 4 | Flower/ |
| | synthase mRNA, complete cds | | | Trichome |
| KY014558.1 | Cannabis sativa isolate Purple_Kush_TPS13 | TPS13PK | 1, 2 | Flower/ |
| | terpene synthase mRNA, complete cds | | | Trichome |
| KY014559.1 | Cannabis sativa isolate Purple_Kush_TPS12 | TPS12PK | 4 | Root/Shoot |
| | terpene synthase mRNA, complete cds | | | |
| KY014560.1 | Cannabis sativa isolate Finola_TPS5 terpene | TPS5FN | 4 | Root/Shoot |
| | synthase mRNA, complete cds | | | |
| KY014561.1 | Cannabis sativa isolate Finola_TPS3 terpene | TPS3FN | 4 | Flower/ |
| | synthase mRNA, complete cds | | | Trichome |
| KY014562.1 | Cannabis sativa isolate Finola_TPS11 | TPS11FN | 4 | Flower/ |
| | terpene synthase mRNA, complete cds | | | Trichome |
| KY014563.1 | Cannabis sativa isolate Finola_CsTPS6 | TPS6FN | 4 | Flower/ |
| | terpene synthase mRNA, complete cds | | | Trichome |
| KY014564.1 | Cannabis sativa isolate Finola_TPS4 terpene | TPS4FN | 4 | Flower/ |
| | synthase mRNA, complete cds | | | Trichome |
| KY014565.1 | Cannabis sativa isolate Finola_TPS2 terpene | TPS2FN | 4 | Flower/ |
| | synthase mRNA, complete cds | | | Trichome |
| KY014566.1 | Cannabis sativa MPDC mRNA, partial cds | MPDC | 1,2 | Root |
| KY014567.1 | Cannabis sativa GPPS small subunit mRNA, | GPPS | 4 | Flower/ |
| | partial cds | | | Trichome |
| KY014568.1 | Cannabis sativa DXR mRNA, partial cds | DXR | 4 | Flower/ |
| | | | | Trichome |
| KY014569.1 | Cannabis sativa IDI mRNA, partial cds | IDI | 1, 2 | Flower |
| KY014570.1 | Cannabis sativa HDS mRNA, partial cds | HDS | 4 | Flower/ |
| | | | | Trichome |
| KY014571.1 | Cannabis sativa FPPS1 mRNA, partial cds | FPPS1 | 1, 2 | Root |
| KY014572.1 | Cannabis sativa HMGR1 mRNA, partial cds | HMGR1 | 1, 2 | Root |
| KY014574.1 | Cannabis sativa MVA kinase mRNA, partial | MVAK | | |
| | cds | | | |
| KY014575.1 | Cannabis sativa CMK mRNA, partial cds | CMK | 4 | Root |
| KY014576.1 | Cannabis sativa DXPS1 mRNA, partial cds | DXPS1 | 4 | Flower/ |
| | | | | Trichome |
| KY014577.1 | Cannabis sativa DXS2 mRNA, partial cds | DXS2 | 4 | Root |
| KY014578.1 | Cannabis sativa MCT mRNA, partial cds | MCT | | |
| KY014579.1 | Cannabis sativa HDR mRNA, partial cds | HDR | 4 | Flower/ |
| | | | | Trichome |
| KY014580.1 | Cannabis sativa FPPS2 mRNA, partial cds | FPPS2 | 1, 2 | Flower/ |
| | | | | Trichome |
| KY014581.1 | Cannabis sativa PMK mRNA, partial cds | PMK | 1, 2 | Root |
| KY014582.1 | Cannabis sativa HMGS mRNA, partial cds | HMGS | 1, 2 | Flower |

The relative level of expression for the identified candidate transcripts of interest in each tissue type is represented in FIG. 6A. It was found that most of these genes involved in terpene synthesis had high expression in the female floral tissues, especially trichomes with some exceptions. For instance, root tissues were found to have higher expression of cannabis 1-deoxy-D-xylulose 6-phosphate (DOXP) synthase (DXS2) involved in MEP pathway; cannabis HMG-CoA reductase (HMGR1), cannabis mevalonate kinase (CMK), cannabis mevalonate-5-phosphate decarboxylase (MPDC), cannabis phospho-mevalonate kinase (PMK) involved in MEV pathway and prenyltransferase cannabis farnesyl diphosphate (FPP) synthase (FPPS1). Whereas, trichomes exhibited higher expression of DXS1, HMGR2 and FPPS2. Additionally, the majority of terpene synthase genes were highly expressed in the female flowers with some outliers. The relative expression analysis revealed TPS5FN, TPS9FN and TPS12PK were more likely to express at heightened levels in the vegetative root and/or shoot tissues. Genes representing CBDAS and THCAS were found to have higher expression in the trichomes; whilst, CBDAS-like 1 was found to have highest expression in the male flower.

Trichomes were found to be significantly enriched in terms of expression for the genes of interest therefore, the relative expression level of these genes was analysed in trichomes across the developmental stages (FIG. 6B). The analysis revealed that the majority of the genes involved in the MEP pathway had high expression levels at Stage 4 of flowering; whereas, the majority of the MEV pathway genes have relatively higher expression during the earlier stages of flower development (Stage 2 and Stage 1). Prenyltransferases (except FPPS1), the majority of terpene synthases (except TPS4FN, TPS5FN that had variable expression and TPS13PK had high expression in Stage 1), CBDAS and THCAS genes also had relatively higher expression in the latter stages of female flower development (mature floral buds) compared to immature floral buds.

### Example 5

### Resequencing of Additional Cannabis Strains

A set of 126 various Cannabis sativa strains were whole genome resequences to identify variants within the gene sequences of the transcriptome. The DNA sequence data was referenced aligned to the transcriptome assembly and transcripts described in Table 3. Variant sequences of the transcripts are described in Table 4. Variant bases of SEQ ID NO: 313-521 are indicated in accordance with the International Union of Pure and Applied Chemistry degenerate base nucleic acid notation.

Of the 312 transcripts analysed, a total of 209 transcripts were identified as containing variants.

### Discussion

Regulation of gene expression plays a significant role in plant growth and development. Comprehensive information on gene expression is required for understanding molecular mechanisms fundamental to any developmental process. Flower development is a key feature for the majority of plants, defining the reproductive phase of the plant and is of even more significance in cannabis, due to cannabinoid production. The current study provides a global view of gene expression dynamics during female cannabis flower development and tissue-specific expression using RNA sequencing. In fact, the number of raw reads generated using RNA sequencing (c. 7 billion) represents a significant advance in coverage compared to those previously published in this species (van Bakal supra; Gao et al. Int. J. Genomics. 2018, 2018: 1-13; Guerriero et al. Scientific Reports. 2017, 7: 4961).

Tissues fell into four major clusters based on the transcriptional activity. The tissues that were included in these major groups represented similar plant structures. Trichomes displayed the least divergence from female flowers which is likely due to the impracticality of removing the trichomes from female flowers in this study. Specific genes were identified that were preferentially tissue expressed and differentially expressed from immature to mature buds in female flowers.

Changes in the gene expression levels during every developmental stage of female flowers and trichomes (especially Stage 1 which is the immature bud to all other stages), indicated that the flower development may be controlled by complex transcriptional regulation. Differential expression between Stage 1 and Stage 4 revealed an enrichment in the “catalytic activity” and “binding” within the GO molecular function category. The GO molecular function categorisation was found to be consistent with a specialized role in defence and specifically in chemical defence as the process is heavily dependent on catalytic activity essential for the production of flavonoids, phenolics, glucosinolates, terpenoids, and alkaloids. Furthermore, the GO biological process category indicated enrichment in “metabolic process” and “cellular process”. The GO category of cellular component revealed that the differentially expressed genes were most frequent for “cell”, “cell part”, “organelle”, and “membrane” during floral bud differentiation. Combining the changes observed in GO terms broadly, a clear picture of cellular turnover in metabolism and defence related compounds emerges that clearly involves a significant number of genes and their related proteins.

Expression profiles of the key aspect of cannabis, cannabinoid and terpenoid synthesis, were analysed across tissue types and developmental stages of female flowers, demonstrating that TPS genes and MEP and MEV pathways' gene transcripts were expressed in floral trichomes at a high level. In addition to this, vegetative root and shoot tissues were found to have high expression of certain terpene synthases (TPS5FN, TPS9FN and TPS12PK) when compared to female flowering tissues. Given that terpene and cannabinoids profile varies based on the developmental stage, the use of gene expression analysis can be adapted to standardise the harvest of female floral buds for resin production. For instance, the majority of the terpene synthases were highly expressed in mature floral buds, expression of TPS13PK (encoding major product, (Z)-(3-ocimene) was found to be highest in immature floral buds when compared to mature buds.

1. A method for determining the sex of a cannabis plant, the method comprising:
a. providing a nucleic acid sample obtained from cannabis plant tissue;
b. determining the level of expression of one or more Cannabis sativa genes, or homologs thereof, wherein the one or more genes encode a gene product selected from the group consisting of:
i. lipoxygenase,
ii. cannabinoid synthesis protein,
iii. geranyl diphosphate pathway protein,
iv. plastidial methylerythritol phosphate (MEP) pathway protein,
v. terpene synthesis protein,
vi. MADs box floral initiation transcription factor,
vii. cannabis allergens, and
viii. leucine-rich repeat (LRR) containing protein;
c. comparing the level of expression determined in (b) with a sex determination reference value; and
d. determining the sex of the cannabis plant based on the comparison made in (c).

2. The method of claim 1, wherein the sex determination reference value is representative of a level of expression of the one or more genes encoding gene products (i)-(viii) in cannabis plant tissue of a male cannabis plant or a plurality of male cannabis plants.

3-33. (canceled)

34. The method of claim 2, wherein one or both of the following applies:
a. a level of expression of the one or more genes encoding gene products (i)-(vi) that exceeds the sex determination reference value is indicative that the cannabis plant is a female cannabis plant; or
b. a level of expression of the one or more genes encoding gene products (vii)-(viii) that is equal to or less than the sex determination reference value is indicative that the cannabis plant is a female cannabis plant.

35. The method of claim 1, wherein one or more of the following applies:
a. the cannabis allergen is selected from the group consisting of Betv1-like protein, pollen allergen, yes allergen, V5 allergen, and Par allergen;
b. the cannabinoid synthesis protein is selected from the group consisting of THCA synthase, cannabidiolic acid synthase, olivetolic acid cyclase, polyketide synthase, chalcone synthase and 2-acylpholoroglucinol 4-prenyltansferase;
c. the MEP pathway protein is selected from the group consisting of deoxyxyluose-5-phosphate synthase, 4-hydroxy-3-methylbut-2-en-1-yl diphosphate synthase, HDS, HDR, 4-hydroxy-3-methylbut-2-enyl diphosphate reductase, C-methyl-D-erythritol 2,4-cyclodiphosphate synthase, fatty acid desaturase, FAD2 and omega-6 fatty acid desaturase; and
d. the terpene synthesis protein is selected from the group consisting of terpene synthase, terpene cyclase/mutase, (−)-limonene synthase, (+)-alpha-pinene synthase, 3,5,7-trioxododecanoyl-CoA synthase, lupeol synthase, secologanin synthase and vinorine synthase.

36. The method of claim 1, wherein the nucleic acid sample is ribonucleic acid (RNA).

37. The method of claim 1, wherein the cannabis plant tissue is selected from the group consisting of inflorescence, shoot, leaf, and root.

38. The method of claim 37, wherein the cannabis plant tissue is inflorescence.

39. The method of claim 38, wherein the cannabis plant tissue is developmental Stage 1 inflorescence.

40. A method for determining the developmental stage of a female cannabis plant inflorescence, the method comprising:
a. providing a nucleic acid sample obtained from female cannabis plant inflorescence or a part thereof;
b. determining the level of expression of one or more Cannabis sativa genes, or homologs thereof, wherein the one or more genes encode a gene product selected from the group consisting of:
ix. cannabinoid synthesis protein,
x. terpene synthesis protein,
xi. plastidial methylerythritol phosphate (MEP) pathway protein,
xii. cytostolic mevalonate (MEV) pathway protein, and
xiii. MADs box floral initiation transcription factor;
c. comparing the level of expression determined in (b) with a developmental reference value; and
d. determining the developmental stage of the inflorescence based on the comparison made in (c).

41. The method of claim 40, wherein the developmental reference value is representative of a level of expression of the one or more genes encoding gene products (i)-(v) in a female cannabis inflorescence at developmental Stage 1 or a plurality of female cannabis inflorescence at developmental Stage 1.

42. The method of claim 41, wherein one or both of the following applies:
a. a level of expression of the one or more genes encoding gene products (i)-(v) that exceeds the developmental reference value is indicative that the inflorescence is at developmental Stage 4; or
b. a level of expression of the genes encoding gene products (v) that is equal to or less than the developmental reference value is indicative that the inflorescence is at developmental Stage 4.

43. The method of claim 40, wherein one or more of the following applies:
a. the cannabinoid synthesis protein is selected from the group consisting of THCA synthase and polyketide synthase;
b. the terpene synthesis protein is selected from the group consisting of terpene syclase, terpene synthase, (−)-limonene synthase, (+)-alpha-pinene synthase, lupeol synthase, vinorine synthase and germacrene-A synthase;
c. the MEP pathway protein is selected from the group consisting of HDR, fatty acid desaturase, delta-12 fatty acid desaturase, omega-6 fatty acid desaturase, delta-12-acyl-lipid desaturase, delta-12-oleic acid desaturase, delta-12 desaturase, delta-12-olate desaturase and delta-12-acyl-lipid desaturase; and
d. the MEV pathway protein is selected from the group consisting of 3-hydroxy-3-methylglutaryl coenzyme A reductase and 4-hydroxy-3-methylbut-2-enyl diphosphate reductase.

44. The method of claim 40, wherein the nucleic acid sample is RNA.

45. The method of claim 40, wherein the nucleic acid sample is obtained from trichome.

46. A method for monitoring the development female cannabis plant inflorescence, the method comprising:
a. determining the developmental stage of a first inflorescence from a female cannabis plant in accordance with the method of claim 40;
b. determining the developmental stage of a second inflorescence from the plant of (a) in accordance with the method of claim 40 at a subsequent time point in the growth cycle of the plant; and
c. comparing the developmental stage determined at (a) and (b) to evaluate whether there has been a change in the developmental stage of the inflorescence.

47. A method for standardising the harvesting of female cannabis plants, the method comprising:
a. determining the developmental stage of an inflorescence from a female cannabis plant in accordance with the method of claim 40;
b. determining the developmental stage of an inflorescence from one or more additional female cannabis plants in accordance with the method of claim 40;
c. comparing the developmental stage determined at (a) and (b) to evaluate if the female cannabis plants have inflorescence at the same developmental stage;
d. optionally, determining the developmental stage additional inflorescence from the plants of (a) and (b) in accordance with the method of claim 40 at a subsequent time point in the growth cycle of the plants; and
e. harvesting the plants when the inflorescence are determined to be at the same developmental stage.

48. The method of claim 47, wherein the plants are harvested at developmental Stage 4.

49. A method for selecting a female cannabis plant for harvest, wherein the female cannabis plant produces inflorescence comprising a cannabinoid profile enriched for total CBD and total THC, the method comprising:
a. determining the developmental stage of an inflorescence from a female cannabis plant in accordance with the method of claim 40;
b. optionally, determining the developmental stage of an additional inflorescence from the plant of (a) in accordance with the method of claim 40 at a subsequent time point in the growth cycle of the plant;
c. harvesting the plant when the inflorescence are determined to be at developmental Stage 4,
wherein the cannabinoid profile comprises a level of total CBD and a level of total THC at a ratio of from about 1:1 to about 5:1 (CBD:THC), wherein the total CDB comprises cannabidiol (CBD) and/or cannabidiolic acid (CBDA), and wherein the total THC comprises Δ-9-tetrahydrocannabinol (THC) and/or Δ-9-tetrahydrocannabinolic acid (THCA), and wherein the level of total CBD and total THC (CBD+THC) is greater than the level of a reference cannabinoid selected from the group consisting of:
d. total CBC, wherein total CBC comprises cannabichromene (CBC) and/or cannabichromene acid (CBCA), and wherein CBD+THC is present at a ratio of from about 10:1 to about 50:1 to the level of total CBC (CBD+THC:CBC);
e. total CBG, wherein the total CBG comprises cannabigerol (CBG) and/or cannabigerolic acid (CBGA), and wherein CBD+THC is present at a ratio of from about 10:1 to about 110:1 to the level of total CBG (CBD+THC:CBG);
f. total CBN, wherein the total CBN comprises cannabinol (CBN) and/or cannabinolic acid (CBNA), and wherein CBD+THC is present at a ratio of from about 400:1 to about 4000:1 to the level of total CBN (CBD+THC:CBN);
g. total THCV, wherein the total THCV comprises tetrahydrocannabivarin (THCV) and/or tetrahydrocannabivarinic acid (THCVA), and wherein CBD+THC is present at a ratio of from about 100:1 to about 600:1 to the level of total THCV (CBD+THC:THCV); and
h. total CBDV, wherein the total CBDV comprises cannabidivarin (CBDV) and/or cannabidivarinic acid (CBDVA), and wherein CBD+THC is present at a ratio of from about 100:1 to about 2000:1 to the level of CBDV (CBD+THC:CBDV).

50. The method of claim 49, wherein the inflorescence further comprises one or more terpenes selected from the group consisting of α-phellandrene, α-pinene, camphene, β-pinene, myrcene, limonene, eucalyptol, γ-terpinene, linalool, γ-elemene, humulene, nerolidol, guaia-3,9-diene and caryophyllene.
